SEQUENCE LISTING (112 sequences)

SEQ ID NO: 1 - protein, 368 aa, Synthetic construct
MPVLRILHLVGSAQDDFYCDLSRLYAQDCLAAMAELPYDSAIAYITPDGQWRFPRSLSREDIAQAKPMPVSEAIEFIAAQNIDIVLPQMFCIPGMTYYRALFDLLEIPYIGNTPDLMAITAHKARTKAIVEAAGVKVPRGEVLRRGDVPTITPPVVIKPVSSDNSLGVTLVKDAAEYEAALEKAFEHGDEAIVETFIEGREVRCGIIVKDGELIGLPLEEYLIDSQEKPIRTYADKLKKTDDGSLGFAAKGNNKSWILDPNDPITQKVQEVAKKCHQALGCRHYSLFDFRIDSQGQPWFLEAGLYCSFAPKSVISSMAKAVGIPLNELLTIAIAETLGSNKYSDRISVVEINEPSKTPRKERELSQMI

SEQ ID NO: 2 - protein, 257 aa, Synthetic construct
MASLENQIILITGASSGIGTACAKIFAGAGAKLILAARRLERLQQLADILTQDFNTEVHLLELDVRDRSAVESAISNLPASWSDIDILINNAGLSRGLDKLHEGSFTDWEEMIDTNIKGLLYLSRYVVPGMVSRGRGHVVNLGSIAGHQTYPGGNVYCATKAAVRAISEGLKQDLLGTPVRVTSVDPGMVETEFSQVRFHGNAQRANQVYQGVTPLTPDDVADVIFFCVTRSPHVNINEVVLMPVDQASATLVNRRT

SEQ ID NO: 3 - protein, 267 aa, Synthetic construct
MLKVDTLKISSQQVEAFERDGVICVKNALDDIWVERLRTAVDRNISIPGPLEEKNAPRPEGSVEHASSLWLVDADFRALAFESPLPTLAAQVLKSEKLNFLADGFFVKKPKTNGHIGWHNDLPYWPVQGWQCCKIWLPLDTVKQENGRLEYIKGSHQWGKELRERSNPSWFVEPEPHEILSWDMEAGDCLIHHFLTIHHSVTNISSTQRRAIVTNWTGDDVTYYQRPKAWPFKPLEEIDLPEFNSFKTKKVGEPIDCDIFPRVEVFR

SEQ ID NO: 4 - protein, 267 aa, Synthetic construct
MLKLELPKITLQEIEAFEQDGVICVKNVLDNIWVERMRKAVDKNISIAGPLEVKGISKPEGNVEHTNSLWLVDADFRALVFESPLATLAAQILKSTKLNFLADGFFVKQPKATSRVGWHNDLPYWPIQGWQCCKIWLALDKVNQQNGRLEYIKGSHRWGKELREDSNPAWFSQPESHELLSWDMEPGDCLVHHLLTIHHSVTNISSTQRRAVVTNWTGDDVTYYPRPKAWPFRPLDEIDIPEFDSLKAKKPGEPIDCDMFPKIKWHR

SEQ ID NO: 5 - protein, 286 aa, Synthetic construct
MLIANSSKISRQEVENFKRDGVICLKNVVDDYWVERMRKAVDRNLLNSNGVRGRKLKTGDVVHDYGLWLKDNDFRDLVFKSPLARVAAQIMESETINFLCDGFFVKKAKADSHVGWHNDLPYWPVKGWKCCKIWLALDPVNQENGRLEYIKGSHLWNKDLRENSNVSWFSEPSYSDILYWDMEPGDALVHHFQTIHHSIGNTTYKSRRAIVTNWTGDDVVYDPSPQTWPFQPIEEIGISEFNSLDTLRSGESIDCEIFPKIDLTPSPSPTSRGEQNPNFLKFPHRL

SEQ ID NO: 6 - protein, 267 aa, Synthetic construct
MLKVDTSKITTQQVEAFERDGVICVKNVLDDIWVERMRRAVDKNVLIPGPLEVKGIPRAEGHVEHTSSLWLTDADFRALAFESPLATLTAQVLKSKKLNFLGDGFFVKKPKGETGVGWHNDKSYWPIQGWQCCKIWLALDSVNQENGKLEYIKASHLWGKELREASDPSWFVEPEPHEIISWDMEPGDCLVHHFMTIHHSVRNTSSTRRRAVVINWTGDDVTYERRPNAWPFRPLEEIDIPEFESLKAKKSGEPIDCDIFPRVELHR

SEQ ID NO: 7 - protein, 272 aa, Synthetic construct
MLKVDTPKISPQQVEAFERDGVICVKNALDDIWIERMRKAVDKNISIPGPLEGKNTPKKEASAEHTSSLWLVDADFRALAFESPLPKLAVGVLKSEKLNFLADGFFVKRPEANGRIGWHNDLPYWPVQGWQCCKIWLALDTVKQENGRLEYIKGSHQWGRELRERSNPSWFVEPEPHEILSWDMEAGDCLIHHFLTIHHSVTNKSSTQRRAIVTNWTGDDVTYYQRPKAWPFKPLEEIDLPQFNSLKTKKFGEPIDCDIFPRVEVHRHRTHI

SEQ ID NO: 8 - protein, 267 aa, Synthetic construct
MLKIDTLKISLQQIEAFERDGVICLRNVLDESWVERMRTAVDKNVSIPGPLEVKGISRPEASVEHTSSLWLVDPDFRALVFESPLSTIAAQLLRSEKLNFLADGFFVKKPKATSRVGWHNDLPYWPIQGWQFCKIWLALDNVNEENGRLEYIKGSHQWGKELREDSNPSWFVEPEPHELLSWDMEPGDCLVHHLLTIHHSVTNISSRQRRAVVTNWTGDDVTYYPRLKAWPFRPLEEIDLPEFNSLKTKKTGEQIDCYMFPPIQLHR

SEQ ID NO: 9 - protein, 267 aa, Synthetic construct
MLKVDTQKISPQQVEAFERDGVICVKNAVDDIWVERMRTAVDKNISIPGPLEDKNVPKPQGSAEHASSIWLIDADFRALAFESPLPTLAAQVLKSKKLNFLADGFFVKKPESNGRIGWHNDLPYWPVQGWQCCKIWLALDTVKQENGRLEYIKGSHQWGKELRERSNPSWFIEPEPHEILSWDMEAGDCLIHHFLTIHHSVTNISSTQRRAIVTNWTGDDVTYYQRPKAWPFKPLEEIDLPEFNSLKTKKSGEPIDCDIFPRVQVHR

SEQ ID NO: 10 - protein, 267 aa, Synthetic construct
MLKLDLPKITLQEIEAFEQDGVICVKNVLDNIWVERMRKAVDKNLSIAGPLEVKGITKPEGNVEHSNSLWLVDTDFRALVFESPLANLAAQFLKSTKLNFLADGFFVKQPKASSRVGWHNDLPYWPIQGWQCCKIWLALDKVNQQNGRLEYIKGSHRWGKELREDSNPSWFSEPEPHELLSWDMEPGDCLVHHLLTIHHSVTNISSTKRRAVVTNWTGDDVTHYPRPKAWPFRPLDEIDIPEFDSLKAKKPGEPIDCDMFPKIKWHR

SEQ ID NO: 11 - protein, 266 aa, Synthetic construct
MLKIDTSKISFQQIGAFERDGVICLRNVLDENWVERMRTAVDKNVSINGPLEAKGISRAEASVEHTSSLWLVDPDFRALVFESPLSTIAAQLLQSEKLNFLADGFFVKKPKATSRVGWHNDLPYWPIQGWQCCKIWLALDHVNEKNGRLEYIKGSHKWGKELREDSNPLWFVEPEPHELLSWNMEPGDCLVHHLLTIHHSVTNISSTQRRAVVTNWTGDDVTYYPRPKAWPFRSVEEIDLPEFNSLKTKKTGEPIDCDMFPQVQLH

SEQ ID NO: 12 - protein, 267 aa, Synthetic construct
MLKVDTRKISHQQVEAFERDGVICVKNAVDDIWVQRMRTAVDKNVLIPGPLEEKNAPKPEASAEHTSNLWLVDADFRALAFESPLPTLAVQVLKSKKLNFLADGFFVKKPKSNSRIGWHNDLPYWPIQGWQCCKIWLALDTVNQENGRLEYIKGSHRWGKELRERSNPSWFVEPKPHEILSWDMEAGDCLIHHFLTIHHSVTNISSRQRRAVVTNWTGDDVTYYQRPKAWPFKSIEEIDLPQFNSFKTKKSGEPLDCDIFPRIEVHR

SEQ ID NO: 13 - protein, 442 aa, Synthetic construct
MSGVRVHRIWDAGPGRTVAALAALCATLPVDLAVVLVALLVGRQPPRGRLPAEARRTVLLNGGKMTKALQLARSFHLAGHRVILVESAKYRWTGHRFSRAVDAFYCVPEPGTPGYAPALLNIVRYENVDVYVPVSSPAGSVPDAVARELLDGACDVVHSDAKTVQLLDDKAEFASTAASLSLQVPDSHRITDARQVADFPFPPGRSYILKRIAYNPVGRMNLTRLSAATPDRNAAYARSLSISEDDPWILQEFIEGREYCTHGTARSGRLQVYGCCESSAAQVNYRSVDKPEIRRWVETFVKNLNLSGQVSFDFIEAHDGQVYAIECNPRTHSAITMFHDHPDLAAAYLNDGHPLITPKHNSRPTYWIYHELWRLLRHPGRLGRLATILRGTDAIFTGWDPVPYLMVHHLQIPALLWANLRVGKGWSRIDFNIGKLVENGGD

SEQ ID NO: 14 - protein, 429 aa, Synthetic construct
MGRTLATLVVLFGTLPFDLALVLVALLAGRRPSRGRLPAQARRTILLNGGKMTKALQLARSFHLAGHRVILVESEKYRWTGHRFSRAVDAFYCVPEPTEPGYALALLDIVRYENVDVYVPVSSPAGSVPDAVARELLDGACDVVHSDAKTVQLLDDKAEFASTAASLSLRVPDSHRITDARQVVDFAFPAGRSYILKRIAYDPVGRMNLTRLSGATPDHNAAYARSLPISEDDPWILQEFIEGREYCTHGTARSGRLQVYGCCESSSAQVNYRNVDKPEIRRWVETFVKNLNLSGQVSFDFIEARDGQVYAIECNPRTHSAITMFHDHPDLAAAYLDDNHPLITPNDGARPTYWIYHELWRLLRHRGRISRLVTMLRGKDAIFAGWDPMPYLMVHHLQIPALLWANLRAGKGWSRIDFNIGKLVENGGD

SEQ ID NO: 15 - protein, 435 aa, Synthetic construct
MREVFQAKTIGTLALLQVVLPLNLALTTFALLRGVFVAPPPVAVAAQRKTILVSGGKMTKALQLARSFHAAGHRVVLVESSKYRFNGHRFSRAVDRFYTVPAPDSDNYAVALLAVVRAEEVDVYVPVCSPVASYYDALAKDQLSPHCEVLHCDADMVARLDDKYEFFALVASLGLSTPETHRVTAPGQVEEFDFTGTDYILKSIPYDPVHRRDMTTVPRPTATETTTYARSKPITEATPWIMQEFVRGQEYCTHSLVRDGAVQVFCCCESSAFQINYRMVDKPEIEEWVGEFAQRLNLTGQVSFDFIQGDDGRLHAIECNPRTHSAITMFYDHPDLARAYLERGVPVVKPLPHSKPTYWIYHELWRLVTQRGGRAHRLAVIAQGKDAIFDWDDPLPFLLVHHLQIPSLLLSNLLRRKGWTGIDFNIGKLIEAAGD

SEQ ID NO: 16 - protein, 530 aa, Synthetic construct
MCRVETRPQVGEHAGMESVPLKAAEGGLVEERKAFLPQSYSLWKDSIEGRLWSLLTLFGLFISSPFLFAFVALSVLSAVVRKLLRLPAARKLPEGSNKGRGRTALVTGGKMTKSLDVCRHLKNEGFRVILTETPRYWMSASRFSSAVDKFVVLPVAPETHPEGYVEALRNLFEKENVSLFAPVCSPFSSLYDAKAAESLPEGAISWSLPAEMVQQLDDKVEFARMAKEVGLPVPDTLRVESKEEVRRFNSELAEKWRRDSSSAIASGAEKKKTDCRRYILKTLDYDPMRRLDLFTLPCGPKELEKYLDETTISPDRPWLVQEFLEGREYSSCALSWKGKLLAFTDNEAVISCYNFKYAGRDKIQEWVRVFCEKYQLSGVICVDFFERADGTQLAIECNPRFSSNMTAFYNNPRLGAAMADPDLALRSGVTETPLPSSKESNWTLVDLYFHSYTQMMKNPLAAFTAAAGLLLVSEETKEKQDAYWAPEDPLPSLALHCFHMPALLVRNVWDGRKWAKIDFCIGKMTEENGD

SEQ ID NO: 17 - protein, 397 aa, Synthetic construct
EVKPNGKVAIVSGGKMTKAYVIARQLKAQGCRVVLLETSKYWMVASRASNCVDRFAMVPLPEKDLAGYLDAVRALAIEEKADLFIPVTSPAASEYEAQVAPVLPAGCVSWSLDLETVRDLDDKTAFCSSAERLGLPAPRSHRVASDEEAHAFNEKLLAEAATATAGAETRYILKSLAYDSMHRLDLFTLPCAPDNPWIIQTFVVGDEYSTCALVKEGRLLAFTDNRACLSCFNYTPARSEALRSWVRDFCAARRLSGVVCIDFIVDAQSGTPYAIECNPRFSSNVLNLFWNPPFGGALFRPHKGGGVEAFFWPPPPPPPLQIWALLSKRPFSLRSAGALLSTVATKKDAYFDVADPLPFIAHLFVHIPALLARNLSTGNKWAKIDPCIGKLTEENGD

SEQ ID NO: 18 - protein, 480 aa, Synthetic construct
MLLPQSITPTMQIFAVFQNLGTLLLLAIAFPFNCIVVLTALLWNLVSKPFRDRGILPVHPKNIMLTGGKMTKALQLARSFHMVGHRVVLVETHKYWLTGHRFSNAVDRFYTVPAPEKDPEAYSQALLAIAKQENIDVYVPVCSPVASYYDSVAKSVLSGCCEVFHFDAEVTQMLDDKYEFAEKARSLGLSVPKSFKITNPEQVINFDFSDAERPYILKSIPYDSVRRLNLTKLPCATPAETAAFVNSLPISPEKPWIMQEFIPGQEYCTHSTVRNGELRMHCCCESSAFQVNYENVDKPEILAWVRHFVKELGITGQASFDFIQAEDGNVYAIECNPRTHSAITMFYNHPGVADAFCRDVTCNTSTSRAGLLNSSFINNISGEPAPTIYPLQPLSTSKPTYWTYHELWRLTGIRSFPQLQTWCKNILRGKDAIFAIDDPLPFLMVHHWQIPLLLLDNLRRLKGWIRIDFNIGKIVELGGD

SEQ ID NO: 19 - protein, 465 aa, Synthetic construct
MTQSISFSSPVPATPPISVKARFIALFQNLGTLTLLLLALPVNAVIVVISLVWNSLTRLFSTQQTTVARSKNILISGGKMTKALQLARSFGAAGHRVVLIETHKYWLSGHRFSNAVSRFYTTPTPQYDPEAYIQTLIDIVKRENIDVYVPVTSPVASYYDSLAKPALSPYCEVLHFDADVTKMLDDKFAFSEKARDLGLSVPKSFKITNPEQVLNFDFSQETRKYILKSIPYDSVRRLDLTKLPCDTLEETAAFVKSLPISPEKPWIMQEFIPGKEFCTHSTVRNGELRLHCCSESSAFQVNYENVENPEIQAWVKHFVNGLGFTGQVSFDFIQTDDGKVYAIECNPRTHSAITMFYNHPQVSDAYLGTEPLTEPLQPLPNSKPTYWLYHEVWRLTGIRSFSQLQNWVRNIFRGTDAIYKLHDPLPFLTVHHWQIPLLLLNNLWQLRGWTKIDFNIGKLVEFGGD

SEQ ID NO: 20 - protein, 471 aa, Synthetic construct
MLCPYERLVFCLKEKLMTQSIPLSFSQPTTPLTVVKTKIVALFKTLGTLALLLLALPLNGFVVLISLLWVIVRNPFTKPTAVAAHPQNILVSGAKMTKALQLARSFHAAGNRVILIEGHKYWLSGHRFSNAVSRFYTVPAPQDDPESYTQALLEIVKKEKIDVYIPVCSPVASYYDSLAKPVLSEYCEVFHFDADITAMLDDKFAFTDQARSLGLSVPKSFKITDPEQIINFDFSQETRKYIIKSISYDSVRRLNLTKLPCDTPEETAAFVRSLPISPEKPWIMQEFIPGKELCTHSTVRDGELRLHCCSNSSAFQINYENVENPQIREWVQHFVKSLRLTGQVSFDFIQAEDGTVYAIECNPRTHSAITMFYNHPGVAQAYLGKTPQAAPLEPLADSKPTYWLYHEIWRLTSIRSWKHLQTWFKNLVRGTDAIYSMDDPIPFLTLHHWQITLLLLQNLQQLKGWVKIDFN

SEQ ID NO: 21 - protein, 465 aa, Synthetic construct
MAQSISLSLPSSTTPSTGVRVKIVALFKTLGTLTLLLIALPFNALIVLIALLWGIARSPFTKKAVVAANPQTILVSGAKMTKALQLARSFHAAGHRVILIEGHKYWLSGHRFSQAVSRFYTVPAPQSDPEAYIQALVEIVKKEKVDIYVPVCSPVASYYDSLAKPTLSEYCEVFHFDADITKMLDDKFAFTDKARSLGLSVPKSFKITDPQQVINFDFSQETRKYILKSIAYDSVRRLDLTKLPCDSPEETAAFVNSLPISPENPWIMQEFIPGKEFCTHSTVRDGELRLHCCCHSSAFQINYENVENPQIREWVQQFVKSLRLTGQVSFDFIQAEDGTVYAIECNPRTHSAITMFYNHPGVAEAYFGKTPLAAPLEPLASSKPTYWIYHEIWRLTNIRSWKQLQTRLNILFRGTDAIFRLNDPVPFLTLHHWQIPLLLLQNLQKLKGWVKIDFNIGKLVELGGD

SEQ ID NO: 22 - protein, 457 aa, Synthetic construct
MAQSISLSSPAKTHAPGISASSLKTLGTLTLLLLALPLNASLVLVALLLKSLRPQNVTTEEPKNILISGGKMTKALQLARSFHEQGHRVILLEAHKYWLTGHRFSFAVNKFYTVEAPEKDPEGYIQSLVNIVEKENIDVYVPVCSPVASYYDSLAKKALPQCEVIHCDAEMTQMLDDKYAFAQTAQSFGLSVPKSFKITEPEQVINFDFSQEKRKYILKSIPYDSVRRLDLTKLPCDTPEATAAFVRSLPISPEKPWIMQEFIPGKEYCTHSTVRNGVITLHCCCESSAFQVNYENVDNPKIFEWVSRFVKELGITGQVSFDFIEAEDGNIYAIECNPRTHSAITMFYNHPGVADAYLGTGSNLAEPIQPKSTSKPTYWTYHEVWRLITTRSWSDFVYRFKIITHGKDAIFSWQDPLPFLMNPHWQIFLLLIQNLQKNRGWVRIDFNIGKLVELGGD

SEQ ID NO: 23 - protein, 465 aa, Synthetic construct
MAQSLPLTSAGGATSPTAFVAQVKALFQNIATLTILLLVLPINAAIVLTSLFWSRVSRFVRPQTVVAANRKNILISGGKMTKALQIARSFHAAGHRVVLIETHKYWLSGHRFSDAISRFYTTPTPQYDPEAYIQALLDIVKKENIDVYVPVTSPVASYYDSLAKPALSPYCEVFHFDADVTQMLDDKFAFSEKARSFGLSVPKSFKITNPEQVLNFDFSGETRKYILKSIPYDSVRRLDLTKLPCDTPEETAAFVRSLPISPEKPWIMQEFIPGKEFCTHSTVKNGELRLHCCAESSAFQVNYENVENPKIQEWVRHFVKELGITGQVSFDFIQAEDGTVYAIECNPRTHSAITMFYNHPDVADAYLSEEPFTEPLVPLPNSKPTYWTYHEVWRLTGIHSFAQLQTWIRNFLQGTDAIYQLDDPLPFLMVHHWQIPLLLLNNLRQLKGWTKIDFNIGKLVEIGGD

SEQ ID NO: 24 - protein, 442 aa, Synthetic construct
MRKYIFVVFQNLGTLVLLAIAFPLNCIVVLTSLLWNFLKQPFNKSIVVNPNSKNILIAGARMTKTLQLARSFHAAGHRVIIIDIEKFWSSGNKYSNSVAGFYTVPDPSKDLEGYVESLHAIAKTEKIDFFIPVAIFSVIHYDQGQPPLPDFVEFFHFDADVTKILDDKFAFAETARSFGLSVPKSFKITHPEQVINFDFSHEKRKYILKSIPYDQIRRLNLTKLPCATSAETAAFVNSLPISEENPWIMQEFIPGKEYCTHTTARDGESRMYCCCESSAFQVNYENVDQQEIMQWATHFTKELGKTGQLSFDFIQAEDGTVYAIECNPRTHSAITMFYNHPGVADAYLGKEPLAESLQPLADSKPTYWLYHEVWRLNEIRNFEQLQTWVRNIRRGKEAIFEVSDPLPFLMVHHWQIPLLILDNLRRLKGWIRIDFNMGELIE

SEQ ID NO: 25 - protein, 459 aa, Synthetic construct
MTQSISVASPAPKTQSVPLGLRISALWKNVGTLALLLLVLPINAVIVLVSLLLGHQSQAIATEPKNILISGAKMTKALQLARSFHAAGHRVVLVETHKYWLTGHRFSKAVSRFYTVPTPQSDPEAYTQALLDIVKTENIDVYVPVCSPIASYYDSLAKPVLSKFCEVFHCDADVTQMLDDKYAFAEKARSLGLSVPKSFKITDPEQILNFDFSQEKRQYILKSIPYDSVRRLDLTKLPCETPEATADFVNSLPISPQKPWIMQEFIPGKEYCTHSTVRNGELRMHCCCESSAFQVNYENVDHPQILEWVRHFVKALGITGQVSFDFIQAEDGTIYAIECNPRTHSAITMFYNHPHVADAYLSEIPQLEPIQPLTNSKPTYWTYHEIWRLTGIRSFSQLQTWLKTFFGGKDAIYCFSDPLPFLTVHHWQIPLLLLQNLQQLKGWIRIDFNIGKLVEFGGD

SEQ ID NO: 26 - protein, 481 aa, Synthetic construct
MLLPQSITPTMQIFAVFQNLGTLLLLAIAFPFNCIVVLTALLGNLVSKPFRDRGILPVSHPKNIMLTGGKMTKALQLARSFHMVGHRVVLVETHKYWLTGHRFSNAVDRFYTVPAPEKDPEGYSQALLAIAKQENIDVYVPVCSPVASYYDSVAKSVLSGCCEVFHFDAEVTQMLDDKYEFAEKARSLGLSVPKSFKITNPEQVINFDFSDAERPYILKSIPYDSVRRLNLTKLPCATPAETAAFVNSLPISPEKPWIMQEFIPGQEYCTHSTVRNGELRMHCCCESSAFQVNYENVDKPEILAWVRHFVKELGITGQASFDFIQAEDGNVYAIECNPRTHSAITMFYNHPGVADAFCRDVTCNTSTSRAGLLNSSFINNISGEPAPTIYPLQPLSTSKPTYWTYHELWRLTGIRSFPQLQTWCKNILRGKDAIFAIDDPLPFLMVHHWQIPLLLLDNLRRLKGWIRIDFNIGKIVELGGD

SEQ ID NO: 27 - protein, 464 aa, Synthetic construct
MAQSISVSSSPAIPSFPSETKIAVIIQNLLTLALLLLALPINATIVLVTLLWHTISRPFQQPATKAANPKNILISGGKMTKALQLARSCNAAGHRVVLIETHKYWLSGHRFSQAVDKFYTVPAPQENPERYTQALIDIIKQENIDVYIPVTSPLGSYYDSLAKPLLSKYCEVFHFDADITERLDDKFAFAETARSLGLSVPKSFKITKAEQVLNFDFSQESRKYILKSIPYDSVRRLDLTKLPCATPEETAAFVRSLPISPEKPWIMQEFIPGKEFCTHSTVRDGELRLHCCCESSAFQVNYENVENSQIREWVRHFVKELKLTGQVSFDFIQAEDGKVYAIECNPRTHSAITTFYDHPQVAQAYLDNEPMAQTLQPLPSSKPTYWTYHEVWRLTGIRSLTQFKKWIANIWRGTDAIYKSDDPLPFLMVHHWQIPLLLIKNLRQLKGWTRIDFNIGKLVELGGD

SEQ ID NO: 28 - protein, 448 aa, Synthetic construct
MAQLQSIQASIFAVLQNLGTLALLMIAFPFNCIVVLLSLLLNFLSRPFHKPVILTKNPRNIMIAGARMTKTLQLARSFHAAGHRVILVDTEKFWLSGNQFSHAVAGFYTVPDPHKDLEGYTQALRAIAKKENIDFFIPVAIFAVIYYDSMSQHQLFDCCEVFHFNADVTKMLDDKFAFAEKARSLSLSVPKSFKITAPEQILNFDFSNEKRKYILKSIPYDAVRRLNMTLLPCDTPEQTAAFVKSLPISEEKPWIMQEFIPGKEYCTHSTVRDGKQTIYCCCESSAFQVNYENVDKPEILQWVNHFVKELGLTGQISFDFIQAVDGTVYVIECNPRTHSAITMFYNHPGVADAYLSKQPLAEPLQPLSDSKPTYWLYHEVWRLNEIRSLKQLQTWIKNILRGKDAIFTVNDPLPFLMVHHWQIPLLLLDNLRRLKGWIRIDFNPLLSL

SEQ ID NO: 29 - protein, 448 aa, Synthetic construct
MTNSLILAVLQNLGTLTLLAIAFPFNLTVVVVALVWDSLTRPFQNPKVANPNPKTIMLTGGKMTKSLQLARSFYADGHRVILVESHKYWLVGHRFSRAVDRFYTVPAPNKDPDGYMEGLLAIAKQENVDVYVPVCSPVASYYDSLAKPVLSGCCEVFHFDPDVTQLLDDKFAFAQKAREFGLSVPKSFKITDPQQVIDFDFRGEKRKYILKSIPYDSVRRLNLTKLPCKTPSETAAFVKSLPISEDKPWIMQEFIPGKEYCTHSTVRNGELRLHCCCESSAFQVNYENVDQPDILQWVSRFVQGLNLTGQASFDFIKTEDGIVYAIECNPRTHSAITMFYNHPGVAEAYLSDTPLPEPLQPLPESKPTYWLYHEVWRLNEIRSFGDIRRWFKTVFGGKDAIFQVNDPLPFLMVHHWQIPLLLLDNLRRMQGWIRIDFNIGKLVELGGD

SEQ ID NO: 30 - protein, 461 aa, Synthetic construct
MAQSISFDSSPATPSLGLETKIAAIIQNILTLALLLLALPINAIIVCIALVLGTIFRPQTTKTSNPKNILISGGKMTKALQLARSFHADGHRVVLLETHKYWLTGHRFSQAVDKFYTTPAPQKKPEDYIKALVDIVKRENIDVYIPVTSPVGSYYDSLAKPELSHHCEVFHFDAEITQMLDDKFAMAEKARSLGLSVPKSFKITSGEQVINFDFSRETRKYILKSIAYDSVRRLDLTKLPCATPEETAAFVRKLPISPEKPWIMQEFIPGKEFCTHSTVRDGEIRLHCCCESSAFQVNYENIENPQILEWVRHFVKELKLTGQISFDFIQTEDGQVYAIECNPRTHSAITTFYNHPQVAEAYIGKQPMAETLQPLATSKPTYWTYHEIWRLTGIRSFTQLKTWLKNIWRGTDAILQLHDPLPFLMVHHWQIPLLLLNNLRQLKGWTRIDFNIGKLVEFGGD

SEQ ID NO: 31 - protein, 439 aa, Synthetic construct
MLNKLIAALQNLLTLTALLITLPINLAIVLIASLIGLFQRETIPQSNSPKRILITGGKMTKALQLARSFHAAGHFVVLVETQKYWLTGHQFSNAVDRFYTVPAPKQDSEAFIQALVDIVQRENIDFFVPVTSPIESYYCSLAKPELSKYCEVLHFDVGITQLLDDKFELSEKARSLNLTAPKTYRITDPQQVLDFEFDSSQYILKSIAYNSVHRLDMTKYPLESKAAMKAHLATLPISEDNPWILQEFISGQEYCTHSTVRDGKVRLHCCAKSSAFQVNYEQVENSEIQAWVTTFVKALNLSGQISFDFIESSSGEVYAIECNPRTHSAITMFYNHPDVAKAYLGEPLTVEPIQPLPTSKPTYWTYHEVWRLITGDRPLYRLQTILHGKDAILQTSDPIPFLMVHHWQIPLLLLNNLRHLKGWVRIDFNIGKLVELGGD

SEQ ID NO: 32 - protein, 481 aa, Synthetic construct
MLLPQSITPTMQIFAVFQNLGTLLLLAIAFPFNCIVVLTALLWNLVSKPFRDRGILPVSHPKNIMLTGGKMTKALQLARSFHMVGHRVVLVETHKYWLTGHRFSNAVDRFYTVPAPEKDPEAYSQALLAIAKQENIDVYVPVCSPVASYYDSVAKSVLSGCCEVFHFDAEVTQMLDDKYEFAEKARSLGLSVPKSFKITNPEQVINFDFSDAERPYILKSIPYDSVRRLNLTKLPCATPAETAAFVNSLPISPEKPWIMQEFIPGQEYCTHSTVRNGELRMHCCCESSAFQVNYENVDKPEIIAWVRHFVKELGITGQASFDFIQAEDGNVYAIECNPRTHSAITMFYNHPGVADAFCRDVTCNTSTSRAGLLNSSFINNISGEPAPTIYPLQPLSTSKPTYWTYHELWRLTGIRSFPQLQTWCKNILRGKDAIFAIDDPLPFLMVHHWQIPLLLLDNLRRLKGWIRIDFNIGKIVELGGD

SEQ ID NO: 33 - protein, 449 aa, Synthetic construct
MAQLQSIQASIFAVLQNLGTLALLMIAFPFNCIVVLLSLLLNFLSRPFHKPVILTKNPRNIMIAGARMTKTLQLARSFHAAGHRVILVDTEKFWLSGNQFSHAVAGFYTVPDPHKDLEGYTQALRAIAKKENIDFFIPVAIFAVIYYDLMSQHPLFDCCEVFHFNADVTKMLDDKFAFAEKARLLSLSVPKSFKITAPEQILDFDFSNEKRKYILKSIPYDAVRRLNMTLLPCDTPEQTAAFVKSLPISEEKPWIMQEFIPGKEYCTHSTVRDGKQTIYCCCESSAFQVNYENVDKPEILQWVNHFVKELGLTGQISFDFIQAVDGTVYAIECNPRTHSAITMFYNHPGVADAYLSKQPLAEPLQPLSDSKPTYWLYHEVWRLNEIRSLKQLQTWVKNILRGKDAIFTVNDPLPFLMVHHWQIPLLLLDNLRRLKGWIRIDFNIGELIE

SEQ ID NO: 34 - protein, 465 aa, Synthetic construct
MAQSISLSLAKSPGSSTGVWVKLVALFKTLGTLTLLLIALPFNALIVLISLLWGFVRSPFRQKAVVADHPQTILVSGAKMTKALQLARCFHAAGHRVILIEGHKYWLSGHRFSKAVSGFYTVPAPELDPLGYIQALVEIVKKEKVDVYVPVCSPVASYYDSLAKPALSEYCEVFHFDADVTKMLDDKFAFTDQARSLGLSVPKSFKITDHQQVINFDFSQETHKYILKNIAYDSVRRLNLTKLPCDTPEETAAFVNSLPISEENPWIMQEFIPGKELCTHSTVRDGELRLHCCSDSSAFQINYENVENPQIREWVQHFVKSLALTGQVSFDFIQAESGTVYAIECNPRTHSAITMFYNHPGVAEAYLGKTPLTDLTEPLANSKPTYWIYHEIWRLTGIRSWKQLQTSINTLAQGTDAVYQLDDPIPFLTLHHWQIPLLLLKNLQQLKGWVKIDFNIGKLVELGGD

SEQ ID NO: 35 - protein, 465 aa, Synthetic construct
MAQSISLSLPESTTPATSVGVKIAALFKTLGTLTLLLIALPFNALIVLIALLWGIVRSPFTKKAVVAAHSQTILVSGAKMTKALQLARSFHAAGHRVILIEGHKYWLSGHRFSQAVSRFYTVPAPQSDSEGYIQALVEIVKQEKVDIYVPVCSPIASYYDSLAKPALSEYCEVFHFDADITKMLDDKFAFTDKARSLGLSVPKSFKITDPQQVINFDFSQETRKYILKSIAYDSVRRLDLTKLPCNTSEETAAFVNSLPISPENPWIMQEFIPGKEFCTHSTVRDGELRLHCCCHSSAFQINYENVENPQICEWVQQFVKSLQLTGQVSFDFIQAEDGSVYAIECNPRTHSAITMFYNHHGVADAYFGKTPLAAPLEPLASSKPTYWIYHEIWRLTGIRSWKQLQTSVNTLLRGTDAIYNLNDPVPFLTLHHWQIPLLLLKNLQQLKGWVKIDFNIGKLVELGGD

SEQ ID NO: 36 - protein, 469 aa, Synthetic construct
MAQSSVSVSASQPIAPPTSIGMRFFALFQNLATLTLLLLALPINATIVLTTLLLNILTSPFQKKQTTVVATEKKNILISGGKMTKALQLARFFHSAGHRVILTETHKYWLSGHRFSQSVDKFYTTPVPQKDSQAYTQALIDIINKEGIDIYIPVTSPIASYYDSLAKPALSEYCEVFHIDAATCEMLDDKFAFSEKARSFGLSIPKCFKITNPEQVINFDFSGETRKYILKSIPYDSVRRLDLTKLPCDTPEETEAFVRSLPISPQKPWIMQEFIPGKEYCTHSTVRDGVMRLHCCCESSAFQVNYENVENPKIREWVTHFVKELGVTGQLSFDFIEAEDGNVYAIECNPRTHSAITIFHDQLQQAANAYLSKEPIAAPLQALPNSKPTYWTYHEFWRLNEIRSLSQLGNWIKNMLRGTDAIYTFDDCLPFLMVHHWQIPVLLLKNLSKLKGWTRIDFNIGKLVELGGD

SEQ ID NO: 37 - protein, 465 aa, Synthetic construct
MAKSVSLSLAKSTTPSTDVRLKLVALFKTLGTLTLLLIALPFNGLIVLIALLWGIVQWPLRKKALVAADPRTVLVSGGKMTKALQLARCFHGAGHRVILIETHKYWLSGHKFSRAVSAFYTVPSPQSDPEGYIQSLVAIVKKEKVDFYVPVCSPVASYYDSLAKPALSAYCEVFHFDADITKMLDDKFAFTEQGRSLGLSVPKSFQITDPQQVINFDFSQETRKYILKNIAYDSVRRLNLTKLPCNTPEETAAFVNSLPISAQNPWIMQEFIPGKELCTHSTVRDGELRLHCCSNSSAFQINYQNVENPQIRQWVQQFVKSLGLTGQVSFDFIQAEDGTVYAIECNPRTHSAITMFYNHPGVADAYLGKTPQAAPVEPLANSKPTYWLYHEIWRLTGIRSWKQLQTSVNTLVGGTDAIFCFDDPVPFLTLYHWQIPLLLLKNLQDLKGWVKIDFNIGKLVELDGD

SEQ ID NO: 38 - protein, 464 aa, Synthetic construct
MAQSISVSSSPAIPSFPSETKIAVIIQNLLTLALLLLALPFNATIVLVTLLWHTISRPFQQATTKTANPKNVLISGAKMTKALQLARSFNAAGHRVVLIETHKYWLSGHRFSQAVDKFYTVPAPQENPERYTQALIDIIKQENIDVYVPVTSPLGSYYDSLAKPMLSNYCEVFHFDADITQKLDDKFAFAETARSLGLSVPKSFKITSAEQVLNFDFSQESRKYILKSIPYDSVRRLDLTKLPCATPEETAAFVKSLPISPEKPWIMQEFIPGKEFCTHSTVRNGELRLHCCCESSAFQVNYENVENSQIREWVRHFVKEQKLTGQVSFDFIQAEDGRVYAIECNPRTHSAITTFYDHPQVAQAYLDKEPMAETLQPLPTSKPTYWTYHEVWRLTGIRSFTQLKKWIANIWRGTDAIYKPDDPLPFLMVHHWQIPLLLLKNLRQLKGWTRIDFNIGKLVELGGD

SEQ ID NO: 39 - protein, 437 aa, Synthetic construct
MLALFQNLGTLLLLALAFPFNCIVVLVALLTKPKLPQATVAKAQNILISGGKMTKALQLARSFYAAGHRVVLIETDKYWLTGHRFSRAVDAFYTVPAPQKDPEAYIQALVNIAKKENIDVYIPVCSPISSYYDSLAKPALAGCCEVFHFDADITKMLDDKFAFAQTAQSFGLSVPKSYKITHPQQVLDFDFSTEQNKYILKSIPYDSVRRLNLTKLPCNTRAETAAFVNSLPISEEKPWIMQEFITGKEYCTHSTVRDGELRLHCCCESSAFQVNYENVDQPEILQWVSHFVKQLGVTGQASFDFIRAENGNIYAIECNPRTHSAITMFYNHPGVASAYLSSQPLKPLQPLTDSKPTYWLYHEVWRLNEIRSLQQLQTWFKNIRRGKESIFAFNDPLPFLMVHHWQIPLLLLDNLRRLAGWIRIDFNIGKLVEFGGD

SEQ ID NO: 40 - protein, 440 aa, Synthetic construct
MFSTTFKSLGTLALLKLALPFNLTLVLIASIINIFSTPFKIKKKPNINSKTVLLTGGKMTKALQLARSFYSAGHRVILVETHKYWLSGHRFSVAVDKFFTIPDPVKDKEGYIDGLLDIVKRENVDIFIPVSSPVASYYDSVAKMVLSPYCKVLHFDVEMTLVLDDKASLCQKASSLGLTSPASYLITDVQEILDFDFSKNNHKYILKSIKYDSVYRLNMTQFPFEGMEEYVRSLPISEENPWVMQQFITGQEYCTHSTVLNGKIRLHCCSMSSHFQVNYEHVDNQKIYEWVEEFVGKLNLTGQISFDFIQTDDGTVYPIECNPRTHSAISMFYNHPLVADAYLNDGDDAPITPLESSKPTFWTYHELWRLTEVRSPQDLSQWWQKVTKGQDGIFSWQDPLPFLMVHHWQIPLLLFGNLIKLKPWVKIDFNIGKLVESAGD

SEQ ID NO: 41 - protein, 459 aa, Synthetic construct
MAQSISFDSSPATPSLGLETKIAAIIQNILTLTLLLLALPINTAIVFIYLVVGAIFRPQTSKTSNPKNILISGGKMTKSLQLARSFHAPGHRVVLVETHKYWLTGHRFSQAVDKFYTTPAPQKDPEAYIQALEEIVKRENIDVYIPVTSPVGSYYDSLAKPKLSPHCEVLHFDAEITQMLDDKFAMAEKARSLGLSVPKSFKITSSEQVINFDFSGETRKYILKSIPYDSVRRLDLTKLPCATPEETAAFVRNLPISPEKPWIMQEFIPGKEFCTHSTVRDGEIKLHCCCESSAFQVNYENVENPQILEWVKHFVKELKLTGQISFDFIQTEDGQVYAIECNPRTHSAITAFYNHPLVAEAYIGSVTETLQPLSTSKPTYWTYHEVWRLTGIRSFTQLKTWLHNIWRGTDAILKLDDPLPFLMVHHWQIPLLLLNNLRQLKGWTRIDFNIGKLVELGGD

SEQ ID NO: 42 - protein, 442 aa, Synthetic construct
MRKHIFVVFQNLGTLVLLAIAFPLNCIVVLTSLLWSFIKQPFNKSIVVNPNSKNILIAGARMTKTLQLARSFHAAGHRVIIIDIEKYWLSGNKYSNSVAGFYTVPDPSKDLEGYVETLHAIANTEKIDFFIPVAIFSVIHYDQGKPPLPDCVEFFHFDADVTKILDDKFAFAETARSFGLSVPKSFKITDPEQVLNFDFSQEKRKYILKSIPYDQVRRLNLTKLPCDTKSETAAFVKSLPISEENPWIMQEFIPGKEYCTHTTARDGESRMYCCCESSAFQVNYENVDQREIMQWASHFTKELGKTGQLSFDFIQAEDGTVYAIECNPRTHSAITMFYNHPGVADAYLGKEPLAESLQPLPDSKPTYWLYHEVWRLNEIRSFKQLQTWVRNIRRGKEAIFEVSDPLPFLMVHHWQIPLLILDNLRRLKGWIRIDFNMGELIE

SEQ ID NO: 43 - protein, 455 aa, Synthetic construct
MKSRQTPRERTFALLKSLGTLSLLLLAFPFSLSAVVGALLWSSLASLFQKRRVQAEPKRILLTGAKMTKCLTLARSFHAAGHQVVMVETHKYWLSGNRFSNCVEAFYTVPAPQHDAEGYIQGLLNIVKQEKIDMFIPVSSPVASYYDSLAKPALSPYCEVFAFDAETTKLLDNKFTFNQKAHSVGLSAPKTFLITNPEQVLNFDFAADGSQYILKSIAYDSINRLALLKLPCAPQKMAEYVRSLPISEENPWIMQEFLKGQEYCTHAVVRDGKLLLYACSKSCDFLVNYEHDYNPAILDWVTRFVKELNLTGQICLDFIQAEDGTVYPIECNPRTSTCITMFHDQPKVVADAYLSSGAQASKEPVQPLPDSKPTYWTFHELWRLLTKVKSWKDLQYRLGIIFNGVDPVFHPRDPLPFLGVNHWQIPLLILNNVRQLKGWERIDFNIGKLVQLGGD

SEQ ID NO: 44 - protein, 455 aa, Synthetic construct
MQSGQTTSERTFALLKSLGTLTLLLLAFPFSLSVVVGALLWSSLTSLFQKRRVQVEPKRILLTGAKMTKCLTLARSFHAAGHQVFMVETKKYWLSGNQFSNCVEALYTVPAPQHDAEGYIQGLLNIVKQEKIDMFIPVSSPVASYYDSLAKPALSPYCEVFAFDAETTKLLDNKFTFNQKAHSVGLSAPKTFLITNPEQVLNFDFAADGSQYILKSIAYDSINRLALLKLPCAPEKMAEYVHSLPISAENPWIMQEFLKGQEYCTHAVVRDGKLLLYACSKSCDFLVNYEHDYNPAILDWVTRFVKELNLTGQICLDFIQAEDGTVYPIECNPRTSTCITMFHDQPKVVADAYLSSSAQAPKEPVQPLPESKPTYWTFHELWRLLTKVKSWKDLQYRLGIIFNGVDPVFHPRDPLPFLGVNHWQIPLLILNNVRQLKGWERIDFNIGKLVQLGGD

SEQ ID NO: 45 - protein, 465 aa, Synthetic construct
MAHSISLSSRPATPAISIKALLVALFQNLGTLTILLLVLPINAAIVLISLLWSRLSSPWRSQKAVVATHRKNILISGGKMTKALQLARSFHAAGHRVVLIETHKYWLSGHRFSNAVSRFYTTPTPQHNPEAYIQALLDIVKREKIDVYVPVTSPVASYYDSLAKPALSPYCEVFHFDADVTQMLDDKFAFSEKARALGLSVPKSFKITNPEQVINFDFSQETRKYILKSIPYDSVRRLDLTKLPCDTPEETAAFVRSLPISPEKPWIMQEFIPGKEFCTHSTVKNGELRLHCCSESSAFQVNYENIENPKIQKWVTHFVKELGITGQISFDFIQAEDGTVYAIECNPRTHSAITMFYNHPQVADAYLSQEAFTEPQEPLPNSKPTYWTYHEVWRLTGIRSFAQLQTWIRNFLRGKDAIYQVDDPLPFLMVHHWQIFLLLLDNLRQFRGWTRIDFNIGKLVELGGD

SEQ ID NO: 46 - protein, 450 aa, Synthetic construct
MQIFAVFQNLGTLLLLAIAFPFNCIVVLTALFWNLVSKPFRDRGILPVSHPKNIMLTGGKMTKALQLARSFHMVGHRVVLVETHKYWLTGHRFSNAVDRFYTVPAPEKDPEGYSQALLAIAKQENIDVYVPVCSPVASYYDSVAKSVLSGCCEVFHFDAEVTQMLDDKYEFAEKARSLGLSVPKSFKITNPEQVINFDFSDAERPYILKSIPYDSVRRLNLTKLPCATQAETAAFVNSLPISPEKPWIMQEFIPGQEYCTHSTVRNGELRMHCCCESSAFQVNYENVDKPEILAWVRHFVKELGITGQASFDFIQAEDGNVYAIECNPRTHSAITMFYNHPGVADAFCRDVTCNVSTLYPLQPLSTSKPTYWTYHELWRLTGIRSFPQLQTWFKNILRGKDAIFAIDDPLPFLMVHHWQIPLLLLDNLRRLKGWIRIDFNIGKIVELGGD

SEQ ID NO: 47 - protein, 454 aa, Synthetic construct
MALILFVQGRAYALFQNLGTLILLLIVLPFNFLKVIPSLLWNFISQPFQKKVVAENPKNILITGAKMTKCLQLARSFHAAGHKVFLLEANKYWLSGNRFSNAVTGFYTLPFPQKDWEGYSQGLLEIIKKEKIDVFIPVSSPAGSYYESLAKPLISEHCEVLHFDAEITQLLDNKFTFIEKAKSFGLSVPKSFLITNPEQVLNFDFATDGSKYILKSIPYDSVRRLDMTKLPMNSKAEMEEFVNSLPISEQRPWIMQEFVKGKEYCTHSTVRKGKVRLYCCCESSEFQVNYHHVDRPQIYQWVEKFVRELNITGQISFDFIQTEDGRVYPIECNPRTHSAITTFYDHPGVADAYLKDSKDENEASLIPLPNSKPTYWTYHELWRLTGIRSLGQLKTWINRIFQGTDGIFQINDPLPFLMVHHWQIPLLLLGNLQKLKGWVRIDFNIGKLVELGGD

SEQ ID NO: 48 - protein, 454 aa, Synthetic construct
MGLISGSQKPIYTVLQNLGTLTLLLSVLPFNLLKVLPALLWNFLSKPFQKKLVVENSKNIILTGAKMTKCLQLARSFQAAGHKVFMLETDKYWLSGNRFSNSVTGFYTVPNPKKDWNGYCQKLLDIVKKENIDVFIPVSSAVLNYYESLVKPILSEYCEVLHFDVEITKLLDNKFTFIEKAKSFGLTVPKSFLITKPEQIINFDFATDGSQYILKSIPYDSVRRLNMTKLPMKSVQEMSNFVKSLPINQEKPWIMQEFVKGKEYCTHSTVRKGQIRLHCCCESSEFQVNYEHVDHPQIYEWIEKFVKELNLTGQISFDFIQTEDNRVYPIECNPRTHSAITTFYNHPEVADAYLNDSQNDNESPITPLSNSKPTYWTYHELWRLTAIRSWEQLKAWSKKITAGTDSIFQFNDPLPFLMVHHWQIPLLLLENLKKLKGWVMIDFNIGKLVELEED

SEQ ID NO: 49 - protein, 440 aa, Synthetic construct
MFLTTFKSLGTLALLKLALPFNLTLVLIASIINIFSNPFKIKKKPNINSKTVLLTGGKMTKALQLARSFHSAGHRVILVETHKYWLSGHRFSVAVDKFFTMPNPVKDKEGYIDGLLDIVKRESVDIFIPVSSPVASYYDSVAKMVLSPYCEVLHFDVEMTLVLDDKANLCKKASSLGLTSPASYLITNVQEILDFDFSKNNHKYILKSIKYDSVYRLNMTQFPFEGMEEYVRSLPISEENPWVMQQFITGQEYCTHSTVRNGKIRLHCCSESSHFQVNYKHIDNQKIYEWVEEFVGKLNLTGQISFDFIQTDDGTVYPIECNPRTHSAISMFYNHPLVADAYLNDGDDAPITPLESSKPTFWTYHELWRLTEVRSPQDLSQWWQKVTKGQDGIFSWQDPLPFLMVHHWQIPLLLFGNLMKLKPWVKIDFNIGKLVESAGD

SEQ ID NO: 50 - protein, 459 aa, Synthetic construct
MTQSISVASVGQTTQSVTLGLRISALFKNLATLALLLLVLPINAAIVLVSLLLGSQSQAIATEPKNILISGGKMTKALQLARSFHAAGHRVVLVETHKYWLTGHRFSKAVSRFYTLPTPQSDPEAYTQALLDIVQKENIDVYVPVCSPVASYYDSLAKPVLSKYCEVFHCDADVTQMLDDKYAFVEKARSLGLSVPKSFKITDPEQVSNFDFSQEKRKYILKSIPYDSVRRLDLTKLPCETPEATADFVNSLPISSQKPWIMQEFIPGKEFCTHSTVRNGELRMHCCCESSAFQVNYENVDHPQILEWVRHFVKALGITGQVSFDFIEAQDGTIYAIECNPRTHSAITMFYNHPDVANAYLSEIPQVEPIQPLINSKPTYWTYHEIWRLTGIRSFSQLQTWLKNFFGGKDAIYSLSDPLPFLTVHHWQIPLLLLQNLQQLKGWIRIDFNIGKLVEFGGD

SEQ ID NO: 51 - protein, 458 aa, Synthetic construct
MAQSLPLSSAPATPSLPSQTKIAAIIQNICTLALLLLALPINATIVFISLLVFRPQKVKAANPQTILISGGKMTKALQLARSFHAAGHRVVLVETHKYWLTGHRFSQAVDKFYTVPAPQDNPQAYIQALVDIVKQENIDVYIPVTSPVGSYYDSLAKPELSHYCEVFHFDADITQMLDDKFALTQKARSLGLSVPKSFKITSPEQVINFDFSGETRKYILKSIPYDSVRRLDLTKLPCATPEETAAFVRSLPITPEKPWIMQEFIPGKEFCTHSTVRNGELRLHCCCESSAFQVNYENVNNPQITEWVQHFVKELKLTGQISFDFIQAEDGTVYAIECNPRTHSAITTFYDHPQVAEAYLSQAPTTETIQPLTTSKPTYWTYHEVWRLTGIRSFTQLQRWLGNIWRGTDAIYQPDDPLPFLMVHHWQIPLLLLNNLRRLKGWTRIDFNIGKLVELGGD

SEQ ID NO: 52 - protein, 465 aa, Synthetic construct
MAQSISLSLPESTTPSTSAGVKIVALFKTLGTLTLLLIALPFNALIVLIALLWGIVRRPFTKKAAVAAHPQTILVSGAKMTKALQLARSFHAAGHRVILIEGHKYWLSGHRFSKAVSRFYTVPAPQKDPEGYIQALVEIVKKEKVDVYVPVCSPVASYYDSLAKPALSEYCEVFHFDADITKMLDDKFAFTDKARSLGLSVPKSFKITDPQQVINFDFSQETRKYILKSIAYDSVRRLDLTKLPCDTPEETAAFVNSLPISSENPWIMQEFIPGKEFCTHSTVRDGELRLHCCCNSSAFQINYENVENPQIREWVQQFVKSLRLTGQVSFDFIQAEDGTVYAIECNPRTHSAITMFYNHPGVADAYLGKTPLAAPLEPLASSKPTYWIYHEIWRLTGIRSWKQLQTSINTLLRGTDAICCLDDPVPFLTLHHWQIPLLLLKNLQQLKGWVKIDFNIGKLVELGGD

SEQ ID NO: 53 - protein, 465 aa, Synthetic construct
MAQSISLSLPQSTTPSTGVKVKIVALFKTLGTLTLLLIALPFNALIVLISLLWGIGRSPFTKKAVVATHPQTILVSGAKMTKALQLARSFHAAGHRVILIEGHKYWLSGHRFSKAVSRFYTVPAPQEDPEGYIQALVEIVKQEKVDVYVPVCSPVASYYDSLAKPALSEYCEVFHFDADITKMLDDKFAFTDRARSLGLSVPKSFKITDPQQVINFDFSQEIRKYILKSISYDSVRRLDLTKLPCDTPEQTAAFVNSLPISPEKPWIMQEFIPGKELCTHSTVRNGELRLHCCSNSSAFQINYENVENPRIREWVQHFVKSLGLTGQVSFDFIQAEDGTTYAIECNPRTHSAITMFYNHSGVANAYFGKTLLDAPLEPLASSKPTYWIYHEIWRLTGIRSWKQLQTSVNTIVRGTDAIYCLDDPVPFLTLYHWQIPLLLLKNLQQLKGWVKIDFNIGKLVELGGD

SEQ ID NO: 54 - protein, 462 aa, Synthetic construct
MAQSISLSLPESTTPSTGIRIKIVALFKTLGTLTLLLIALPINALIVLLSLLWSILFTKKPAVAAHPQTILVSGGKMTKALQLARSFHAAGHRVILVEGHKYWLSGHRFSNAVSRFYTVPAPQDDPEGYIQALLEIVKKEKVDIYVPVCSPVASYYDSLAKPSLSAYCEVFHFDAEITKMLDDKFAFTDQARSLGLSVPKSFKITDAEQVINFDFSKETRKYIIKSISYDSVRRLNLTKLPCDTPEETAAFVKSLPISPEKPWIMQEFIPGKELCTHSTVRDGELRLHCCSDSSAFQINYENVENPQIRQWVQHFVKSLGLTGQVSFDFIQAEDGTAYAIECNPRTHSAITMFYNHPGVAEAYFGKTLLAAPLEPLADSKPTYWIYHEIWRLTGIRSAKQLQTWFQRLVRGTDAIYQINDPIPFLTLHHWQITLLLLQNLQKLKGWVKIDFNIGKLVELGGD

SEQ ID NO: 55 - protein, 463 aa, Synthetic construct
MAQSIPFDSASPTPQVSWGVRISALWKTVGTLLLLFLALPVNASIVLISLLWGIFSKPFEKRVVAAAPKNILISGGKMTKALQLARSFHAAGHRVVLVESHKYWLTGHQFSNAVSVFYTVSPPEKDPEGYTQQLLDIVKKERIDVYVPVCSPVASYYDSLVKPALSQHCEVFHCDAEITQMLDDKYAFSEKARSFGLSVPKSFKITNPEQVINFDFSQEKRKYILKSIPYDSVRRLNLTKLPCDTPEETAAFVRSLPISPEKPWIMQEFIPGKEFCTHSTVRNGELRLHCCCESSAFQVNYENVNNPQILEWVKHFIKEMGITGQVSFDFIQTEDGTVYAIECNPRTHSAITMFYNHPGVADAYLGKIPLPEPLQPLADSKPTYWLYHEIWRLTGIRSLSQFWTWLKNLMRGKDAIYQLNDPLPFLTVPHWQITLLLLQNLRQLRGWVKIDFNIGKLVELGGD

SEQ ID NO: 56 - protein, 455 aa, Synthetic construct
MQSGQTIRERTFASLKSLGTLTLLLLAFPFSLSVVVGALLWSSLTSLFQKHRVQVKPKRILLTGAKMTKCLTLARSFHAAGHQVFMVETKKYWLSGNQFSNCVEALYTVPAPQHDAEGYIQGLLNIVKQEKIDMFIPVSSPVASYYDSLAKPALSPYCEVFAFDAETTKLLDNKFTFNQKAHSVGLSAPKTFLITNPEQVLNFDFATDGSQYILKSIAYDSINRLALLKLPCAPATMAKYVHSLPISEENPWIMQEFLKGQEYCTHAVVREGKLMLYACSKSCDFLVNYEHDYNPAILDWVTRFVKALNLTGQICLDFIQAEDGTVYPIECNPRTSTCITMFHDQPKVVADAYLSSSASILKEPVQPLPDSKPTYWTFHELWRLITKVKSWQDLQYRLGIIFNGVDPVFHPRDPLPFLGVNHWQIPLLILNNVRQLKGWERIDFNIGKLVQLGGD

SEQ ID NO: 57 - protein, 454 aa, Synthetic construct
MLIILFIQNHAYALFQNLSTFLLLTLLLPFNLLKILPVVLWNILTPIRAKPPGYEKPKNILITGAKMSKSLQLARSFNGSGHRVFLLEIHKYWLSGNRFSNAIKGFYTVPNPQKDWDGYQQAVLEIVQKENINLFIPVSSPAGSYDESRLKPILSPYCEVFHFNLDITELLDNKFTFIEKAKSLGLSVPQSFLITDSKQILDFDFAQDGSRYILKSIPYDSVRRLDMTKLPMKSEQEMEEFVKKLPITEDKPWIMQEFVQGKEYCTHSTVRKGKIRLHCCCESSEFQVNYDHVEEPEIYQWVETFVRALNLTGQISFDFIKTEDGQVYPIECNPRTHSAITTFHDHPGVADAYLKDAEDETESPIFPLPDSKPTYWTYHELWRVTEIRSFGQFQAWIKRITEGTDGIFQLNDPLPFLMVHHWQIPLLLLQNLKKMKGWVRIDFNIGKLVELDGD

SEQ ID NO: 58 - protein, 472 aa, Synthetic construct
MGQSISLSLPQSPTSSTSVRVKIIALFKTLGTLTLLLIALPFNALIVLISLLWGIVRWTLPRRRRSLFTKNVVAAHPQTILVSGAKMTKALQLARSFHAAGHRVILIEGHKYWLSGHRFSKAVSRFYTVLAPQSDLEGYIQALVEIVKKEKVDVYVPVSSPVSSYYESLAKAALSEYCEVFHFDPDITKMLDDKFALTDRARSLGLSVPKSFKITDPQQVINFDFSQETRKYILKSIDYDSVRRLNLTKLPCDTPEETAAFVNSLPISPEKPWIMQEFIPGKELCTHSTVRDGELRLHCCSDSSAFQINYENVENPQIREWVQHFVKSLALTGQVSFDFIQAQDGTVYAIECNPRTHSAITMFYNHPGVADAYLGKTPLAAPLEPLASSKPTYFIYHEIWRLTGIRSWKQLQTSVNTLVRGTDAIYSLDDPIPFLTLHHWQIPLLLLKNLQQLKGWVKIDFNIGKLVELGGD

SEQ ID NO: 59 - protein, 463 aa, Synthetic construct
MPQSISLTSSPTINQVNNKSVDISSSLKTLGTLTLLLLALPVNATLVLVALLLNSLRPRNITTAANPKNILISGGKMTKALQLARSFHNAGHRVVLLEAHKYWLTGHRFSFAVNKFYTVEAPEKDPEGYVQSLVDIVNKENIDVYVPVCSPVASYYDSLAKKALSSQCEVIHCDALTTQMLDDKYAFTETARGFGLSVPKSFKITDPEQVINFDFSQEKRKYILKSIPYDSVRRLDLTKLPCDTPEATAAFVRSLPISPEKPWIMQEFIPGKEYCTHSTVRNGEITLHCCCESSAFQVNYAQVDNPQIFEWVRHFLKQLGITGQVSFDFIEAEDGTVYAIECNPRTHSAITMFYNHPGVADAYLGTLNNLEEPIQPLPTSKPTYWIYHEMWRLINAGSWSKFVERLQIITRGTDAIFSWQDPLPFLMNPHWQIFLLLIQNLQKNRGWIRIDFNIGKLVELGGD

SEQ ID NO: 60 - protein, 450 aa, Synthetic construct
MFLQAKIWAFFQNIGTLTLLLLALPFNAIVVLPCLLWSWIAKLFQKKVVAANPKNILITGGKMTKALQLARCFHAAGHTVFLVETHKYWLSGHRFSRAVKGFFTVPAPEKHANGYCQGLLDIVKQEKIDVFIPVSSPVASYYDSIAKSLLSPHCEALTFDAEITEMLDNKFTFCQKARELGLTAPKAFLITDPEQVLNFDFAADGSRYILKSIAYNSVYRLDLTKLPMSSKEQMASFVKGLPISESQPWIMQEFISGQEYCTHSTVRNGIVRLHCCSQSSPFQVNYEQVDNQNIFQWVQQFVKALNLTGQISLDVIQTKDGKVYPVECNPRTHTAIAMFYNHPGVADAYILDSKDAREPPIQPLPESKPTYWTYHELWRLTGIRSWGQLKGWFNKIIKGTDGIFQVNDPLPFLMVHHWQIPLLLLNNMRKFKGWVKIDFNIGKLVELGGD

SEQ ID NO: 61 - protein, 465 aa, Synthetic construct
MAQSISLSLPQSPTSSTGIKVKLVALFNTLGTLTLLLIALPFNALIVLISLLWGIVSSPFTKKAVVAAHPQTILVSGAKMTKALQLARSFHAAGHRVILIEGNKYWLSGHRFSKAVSRFYTVPAPQEDPEGYIQALVEIVKREKVDVYVPVCSPVASYYDSLAKPLLSEYCEVFHFDPDITKMLDDKFAFTDRARSLGLSVPKSFKITDPQQVINFDFSQETRKYILKSIDYDSVRRLNLTKLPCDTPEETAAFVNSLPISAEKPWIMQEFIPGKELCTHSTVRNGELRLHCCSNSSAFQINYENVENPQIREWVQHFVKSLALTGQVSFDFIQAEDGTAYAIECNPRTHSAITMFYNHPGVADAYLGKTPLAAPLEPLASSKPTYFLYHEIWRLTGIRSWKQLQTSVNTLVRGTDAIYSLDDPIPFLTLHHWQIPLLLLKNLQQLKGWVKIDFNIGKLVELGGD

SEQ ID NO: 62 - protein, 469 aa, Synthetic construct
MLMGFFEGEFMTQSISVASPAPKTQSVPLGFRISALWKNVGTLALLLLVLPINAVIVLVSLLLGHQSQAIATEPKNILISGGKMTKALQLARSFHAAGHRVVLVETHKYWLTGHRFSKAVSRFYTLPTPQSDPKAYTQALLDIVKKENIDVYVPVCSPVASYYDSLAKPVLSKYCEVFHCDADVTQMLDDKYAFAEKARSLGLSVPKSFKITDPEQVINFDFSQEKRQYILKSIPYDSVRRLDLTKLPCETPEVTADFVNSLPISPQKPWIMQEFIPGKEFCTHSTVRNGELRMHCCCESSAFQVNYENVDHPQILEWVRHFVKELGITGQVSFDFIQAEDGTIYAIECNPRTHSAITMFYNHPSVADAYLSEIPQLEPIQPLFNSKPTYWIYHEIWRLTGIRHWSQLQTWLKNFFGGKDAIYSFSDPLPFLTVHHWQIPLLLLQNLQQLKGWLRIDFNIGKLVEFGGD

SEQ ID NO: 63 - protein, 454 aa, Synthetic construct
MGLISRSQKPVYIALQNLGILTLLLSVLPFNLLKVLPAVLWNFISKPFQKKVVAENSKNIILTGAKMTKCLQLARSFQVAGHKVFMLETDKYWLSGNRFSNTVTGFYTVPNPKKNWNGYCQELLDIVKREDIDVFIPVSGAALNYYESLIKPILSEHCEVLHFDIEITKLLDNKFTFIEKAKSFGLAVPKSFLITNPEQILNFDFPADGGQYILKSIPYDSVRRLDMRKLPMKSAQEMKDFVNSLPISEEKPWIMQEFVKGKEYCTHSTVRKGQIRLHCCCESSEFQVNYEHVNHPQIYEWVETFVKELNLTGQISFDFIQTEDNRVYPIECNPRTHSAITTFYNHPEVADAYLNDSQDDNESPLIPLPNSKPTYWIYHELWRLTAIRSWEQLKDWIKKITAGTDSIFQFNDPLPFLMVHHWQIPLLLLDNLKKLKGWVMIDFNIGKLVELEED

SEQ ID NO: 64 - protein, 464 aa, Synthetic construct
MTQSISLSLPESTTPSVGIKVKILALFKTLGTLSLLLVALPFNVLIVLISLLWGIVRVPFTKNVVATHSQTILVSGAKMTKALQLARSFHADGHRVILIESHKYWLSGHRFSKAVSRFYTVPSPQKDPESYIQALIEIVKKEKVDVYVPVCSPVASYYDSLAKPALSEYCEVFHFNADITKMLDDKFAFTQKARALGLSVPKSFKITDPQQVINFDFSQETRKYILKSINYDSVRRLNLTKLLCDTPEETAAFVKSLPISPETPWIMQEFIPGKEFCTHSTVRDGELRLHCCCHSSAFQINYENVENPQIREWVQHFVKSLGLTGQVSFDFIQAEDGTVYAIECNPRTHSAITMFYNHPGVAEAYFGKIPLPAPVEPLATSKPTYWTYHEIWRLTGIRSWKQLQTAIKTIFQGTDAIYCLDDPLPFLTLHHWQIPLLLLQNLQQLKGWVKIDFNIGKLVELGGD

SEQ ID NO: 65 - protein, 458 aa, Synthetic construct
MAQSLSLSSSHATPSIPWQTRVAAILQNIGTLTLLLLALPINASIVFISWLIFRPQKVKAANPQNILISGGKMTKALQLARSFHAAGHRVVLLETHKYWLTGHRFSVAVDKFYTVPAPQENPQAYIQALVDIVKQENIDVYVPVTSPAGSYYDSLAKPELSRYCEVFHFDADITQMLDDKFALVEKARSLGLSVPKSFKITSPEQVINFDFSGESRKYILKSIPYDSVRRLDLTKLPCATPEETAAFVRTLPISQEKPWIMQEFIPGKEFCTHSTVRDGELRLHCCCESSAFQVNYENVDNPQIREWVRRFVKELKLTGQISFDFIQAEDGTVYAIECNPRTHSAITTFYDHPQVAQAYLSKETTAETLQPLATSKPTYWTYHEVWRLTGIRSLTQLGRWLGNIWRGTDAIYQPGDPLPFLMVHHWQIPLLLLNNLRRLKGWTRIDFNIGKLVELGGD

SEQ ID NO: 66 - protein, 464 aa, Synthetic construct
MLETVSVAAMPSERETNTGNRRFPTAFKTIATLILLLLVMPLNLALTAIALLRSIIIKPFQSRSTTATPQTILISGGKMTKALQLARSFHQAGHRVILVETEKYWLTGHRYSRAVDRFYTVPNPQTEEYPQALLKIVRQEGVNVYVPVCSPVASYYDAEVKRVLSGHCTVMHVDVETLQRLDDKYEFATAAQALGLPVPKSYRITNPQQVIDFDFSDAQRKYIIKSIPYDSVRRLDLTKLPCETPAETAAFVNSLPISESKPWIMQEYIPGQEFCTHSTVRNGHLQLHCCCKSSAFQVNYENVDRPDIENWIRQFAKSLNLTGQVSFDFIQAADDGEIYAIECNPRTHSAITMFYNHPDVAKAYLEPDPLPQTVQPLASSRPTYWIYHEIWRLVTHLSSPKLVSERLKIIAQGKDAIFDWDDPLPFLMVHHWQIPLLLWGNLQNPKEWIRIDFNIGKLVEIGGD

SEQ ID NO: 67 - protein, 347 aa, Synthetic construct
SRSVDRFYTVPKPQEKDYIDALLEIVQREGVDVYIPVCSPVASYYDALAKQVLSKYCEVMHFDPELVQKLDDKSEFSAIATSLGLAVPDSYRITDTQQILDFDFAKQAHTYILKSIPYDSLRRLNLTQLPCETPQQTAAFVEQLPICESNPWIMQAFITGQEYCTHSTVRNGELQLHCCCESSAFQINYEMVDKPEIEAWVRKFVSSLKLTGQVSFDFIQTRDGGVYAIECNPRTHSAITMFYNHPDVARAYLESDFPLIKPLESSRPTYWIYHEIWRLVTQPTQIGQRLKIIASGKDAIFDWADPLPFLMVHHAQIPWLLLENLRQLKGWMRIDFNIGKLVEPAGD

SEQ ID NO: 68 - protein, 455 aa, Synthetic construct
MAQVQPIKARIFAVFQNLGTLALLAIAFPINCIVVLASLLWNFCSRPFSKQGVSTLNPKNILIGGGKMTKTLQLARLFHAAGHRVILFDSEKFRFSGYRFSNAVDRFYTVPDPQTDLEGYTQALRAIAKQENIDIFIPVGIFAGGYFDSQRQPVLSGCCELFHFDADTMKMLDNKFTFGEIARSFGLSVPKTFLITDPEQVLQFDFANEKNKYILKSIVYDSVYRLDMTKLPMESQEKMAAHVNSLPIRKDNPWILQEFISGKEYCTHSTVRNGELTVHCCCESSAFQVNYENVDHPEIMQWVSRFVKELKLSGQISFDFMQAEDGTLYAIECNPRTHSAITMYYNHPDLADAYLSAERRNYALPLQPLPDSKPTYWLYHEVWRLNEIRSLKQLQTWFKNIWRGKDAIFEVNDPLPFLMVHHCYIPLLLLDSLRKLKGWVRIDFNIGKLVQLEGD

SEQ ID NO: 69 - protein, 463 aa, Synthetic construct; identical to SEQ ID NO: 59

SEQ ID NO: 70 - protein, 464 aa, Synthetic construct
MAQSISVSSSPAMPSLAVETKIAVIIQNILTLALLLLALPINATIVVVTLLWCNISRPFQHSATKAANPKNILISGGKMTKALQLARSFNAAGHRVVLIETHKYWLSGHRFSQAVDKFYTVPAPQENPECYTQALIDIIKQENIDVYIPVTSPLGSYYDSLAKPLLSEYCEVFHFDADITQKLDDKFAFAETARSLGLSAPKSFKITSAEQVLNFDFSQESRKYILKSIPYDSVRRLDLTKLPCATPEETAAFVRSLPISPEKPWIMQEFIPGKEFCTHSTVRDGELRLHCCCESSAFQVNYENVENSQIREWVRHFVKELKLTGQISFDFIQAEDGRVYAIECNPRTHSAITTFYDHPKVAQAYLDKEPMAETLQPLPTSQPTYWTYHEVWRLTGIRSFTQLKKWIANIWRGTDAIYKSDDPLPFLMVHHWQIPLLLIDNLRRLKGWTRIDFNIGKLVELGGD

SEQ ID NO: 71 - protein, 458 aa, Synthetic construct; identical to SEQ ID NO: 51

SEQ ID NO: 72 - protein, 459 aa, Synthetic construct
MTQSISVASVGQTTQSVTLGLRISALFKNLATLALLLLVLPINAVIVLVSVLLGSQSQAIATEPKNILISGGKMTKALQLARSFHAAGHRVVLVETHKYWLTGHRFSKAVSRFYTLPTPQSDPQAYTQALLDIVKKESIDVYVPVCSPVASYYDSLAKPVLSKYCEVFHCDADVTQMLDDKYAFAEKARSLGLSVPKSFKITDPEQVINFDFSQEKRQYILKSIPYDSVRRLDLTKLPCETPQATADFVNSLPISPQKPWIMQEFIPGKEYCTHSTVRNGELRMHCCCESSAFQVNYENVDHPQILEWVRHFVKALGITGQVSFDFIEAEDGTIYAIECNPRTHSAITMFYNHPDVANAYLSEIPQVEPIQPLTNSKPTYWTYHEIWRLTGIRSFSQLQTWVKNFFGGKDAIYSLSDPLPFLAVHHWQIPLLLLQNLQQLKGWIRIDFNIGKLVEFGGD

SEQ ID NO: 73 - protein, 450 aa, Synthetic construct
MFLQAKIWAFFQNIGTLTLLLLALPFNAIVVLPCLLWSWIAKLFQKKVVAANPKNILITGGKMTKALQLARCFHAAGHTVFLVETHKYWLSGHRFSRAVKGFFTVPAPEKHANGYCQGLLDIVKQEKIDVFIPVSSPVASYYDSIAKSLLSPHCEALTFDAEITEMLDNKFTFCQKARELGLTAPKAFLITDPEQVLNFDFAADGSRYILKSIAYNSVYRLDLTKLPMSSKEQMASFVKGLPISESQPWIVQEFISGQEYCTHSTVRNGIVRLHCCSQSSPFQVNYEQVDNQKIFQWVQQFVKALNLTGQISLDVIQTKDGKVYPVECNPRTHTAIAMFYNHPGVADAYLLDSKDAREPPIQPLPESKPTYWTYHELWRLTGIRSWGQLKGWFNKIIKGTDGIFQVNDPLPFLMVHHWQIPLLLLNNMRKFKGWVKIDFNIGKLVELGGD

SEQ ID NO: 74 - protein, 423 aa, Synthetic construct
MFPINLTLVITAFLTNLITLPFPKKITYENSKNILLTGGKMTKSLQLARSFHRAGHKVFMVETHKYWLSGHQYSKAVKKFLTVPAPEKDPEGYCQSLLDIVKREKIDVFIPVSSPVASYYDSLAKPILSPYCEVFHFDTEMTKTLDDKFSLCEQARVLGLTAPKVFLITSPGEIINFDFSQEQNPYIIKSIQYDSVTRLDMTKFPFEGMKEYVKKLPISKERPWVMQEFIKGQEYCTHSTVRDGEIRLHCCSKSSPFQVNYEQVDNPEIFQWVQKFVKELNLTGQISFDFMQTEDGKVYPIECNPRTHTAITMFYDHPGLADAYLEPGKNQPHIEPLPTSKPTYWLYHELWRITGIRSFNDLTNWLNKVIKGKDAMLDKDDPLPFLMVHHWQIVLLLLQNMVKLKGWVRIDFNIGKLVEIGGD

SEQ ID NO: 75 - protein, 394 aa, Synthetic construct
LITGAKMSKSLQLARSFNGAGHRVFLLETHKYWLSGNRFSNAIKDFYTVPNSEKNWDGYQQAVLEIVQKENINLFIPVSSAAGSYDESRLKAILSPYCEVFHFDLDITELLDNKFTFIEKAKNLGLSVPKSFLMTDSKQILDFDFVQDGSRYILKSIPYDSVRRLDMTKLPMKSEQEMEEFVKELPITEDKPWIMQEFVQGKEYCTHSTVRKGKIRLYCCCESSEFQVNYNHVEEPEIYQWVKTFVRALNLTGQISFDFIKTEDGQVYPIECNPRTHSAITTFHDHPGVADAYLKDVEDETKSPIFPLPDSKPTYWTYHELWRLTQIRSFGQFKAWIKRMIEGTDGIFQPHDPLPFLMVHHWQIPLLILQNLKTMKGWVRIDFNIGKLVELDGD

SEQ ID NO: 76 - protein, 464 aa, Synthetic construct
MAQSISVDSSPAIPSLASETKIAVIIQNILTLALLLLALPINATIVLVTLFWGTILRPFQHSATKTANPKNILISGGKMTKALQLARSFHAAGHKVVLLETHKYWLTGHRFSQAVDKFYTVPAPQENPESYTQALIDIIKQENIDVYIPVTSPLGSYYDSLAKPLLSRHCEVFHFDVDITQNLDDKFEFAQKARSLNLSAPKSFKITSAEQVLNFDFSQESRKYILKSIPYDSVRRLDLTKLPCATPEETAAFVRSLPISPEKPWIMQEFIPGKEFCTHSTVRDGELRLHCCCESSAFQVNYENVENSQIREWVRHFVKELKLTGQISFDFIQAEDGAVYAIECNPRTHSAITTFYDHPKVAQAYLDQEPMAETLQPLPTSKPTYWTYHEVWRLTGIRSFTQLQKWLANIGRGTDAIYKLDDPLPFLMVHHWQIPLLLLNNLLRLKGWTRIDFNIGKLVELGGD

SEQ ID NO: 77 - protein, 469 aa, Synthetic construct
MAQSSIPVLSSQTATHTISLGRRFVALVQNLATLTALLLALPINATIVFISLVLKILISPFQKEQTTVTTAERKNILISGGKMTKALQLARFFHAAGHRVVLTETHKYWLSGHRFSQAVDKFYTTPVPQKDSQIYTQALIDIVNKENIDIYIPVTSPIASYYDALAKQTLSEYCEVFHIDAATCEMLDDKFAFSEKARSFGLSVPKSFKITNPEQVLNFDFSGETRKYILKSIPYDSVRRLDLTKLPCDTPEETEAFVRSLPISPQKPWIMQEFIPGKEYCTHSTIRDGVVRLHCCCESSAFQVNYENVENAKIREWVTHFVKELGVTGQLSFDFIEAEDGNVYAIECNPRTHSAITIFHDQLQPAANAYLSKEPIKEPLQALINSKPTYWTYHEFWRLNEIRSFSQLGNWIKNMLQGTDAIYTFDDSLPFLMVHHWQIPLLLLKNLFKLKGWTRIDFNIGKLVESGGD

SEQ ID NO: 78 - protein, 465 aa, Synthetic construct
MAQSISLTESQTTVKPLAVWGKINALLKNLGTLVLLLVALPINATIVLVSLLWNLLAKPFQKEQTVAGDRKNILISGAKMTKALQLARSFHAAGHRVVLLETHKYWLSGHRFSKAVDNFYTTPVPQRDPQAYTQALIDIIEKENIDVYIPVTSPIASYYDSLAKPVLSQYCEVFHFDAAVTQMLDDKFAFSEKARSLGLSVPKSFKITSPEQVLNFDFSQETRKYILKSIPYDSVRRLDLTKLPCDTPEQTEAFVRSLPISAQKPWIMQEFIPGKEFCTHSTVRDGEIRLHCCCESSAFQVNYEHVEHPQISEWIARFVKGLGITGQISFDFIQAEDGSVYAIECNPRTHSAITTFHDRPEVAQAYLGKEAMTEPLQPLPSSKPTYWLYHEVWRLTSIRSLAQLRTWIRNIWRGTDAIYKLDDPLPFLMLHHWQIPLLLLNNLWRLKGWTRIDFNIGKLVELGGD

SEQ ID NO: 79 - protein, 453 aa, Synthetic construct
MAQLLFVRTPSFTMLKSLGTLTLLLIAFPINSIVVLTSLLWGLLSRPFQKQPLPADNQKTAMFTGGKMTKALQLARSFHAAGHRVILVETHKYWLTGHRFSNAVDRFYTIPAPQKDPEGYTQALLNIAKQENVDIYIPVCSPVSSYYDSLAKPALSGCCEVFHFDADITKMLDDKFAFSEKARALGLSVPKSFKITNPEQVLNFDFSNETRKYILKSIPYDSVRRLNLTKLPCDTPEETAAFVKSLPISEEKPWIMQEFIPGQEYCTHSTVRDGELRLHCCCESSAFQVNYENVDQPEIMKWVSHFVKELKLTGQASFDFIQAEDGAIYAIECNPRTHSAITMFYNHPGVADAYLGKEPLAEPLQPLPDSKPTYWLYHEIWRLNEIRSWSQLQTWMNNLLRGTDAIFDVNDPLPFLTVHHWQIPVLLLDNLRKLRGWVRIDFNIGKLVESGGD

SEQ ID NO: 80 - protein, 461 aa, Synthetic construct
MAQSISLSLPQSTTPSKGVRLKIAALLKTIGTLILLLIALPLNALIVLISLMCRPFTKKPAVATHPQNILVSGGKMTKALQLARSFHAAGHRVILIEGHKYWLSGHRFSNSVSRFYTVPAPQDDPEGYTQALLEIVKREKIDVYVPVCSPVASYYDSLAKSALSEYCEVFHFDADITKMLDDKFAFTDRARSLGLSAPKSFKITDPEQVINFDFSKETRKYILKSISYDSVRRLNLTKLPCDTPEETAAFVKSLPISPEKPWIMQEFIPGKELCTHSTVRDGELRLHCCSNSSAFQINYENVENPQIQEWVQHFVKSLRLTGQISLDFIQAEDGTAYAIECNPRTHSAITMFYNHPGVAEAYLGKTPLAAPLEPLADSKPTYWIYHEIWRLTGIRSGQQLQTWFGRLVRGTDAIYRLDDPIPFLTLHHWQITLLLLQNLQRLKGWVKIDFNIGKLVELGGD

SEQ ID NO: 81 - protein, 453 aa, Synthetic construct
MTKLQPIKARIIAVFQNLGTLLLLAIAFPINCSVVLVSLLWNFFSRPSHKQVVLTENPKNILIGGGRMTKTLQLARSFHAAGHRVILVDIDKYWLSGHRFSRAVAGYYTVPAPQKDLEGYTQALRAIAKKENIDFFIPVAIFAVSYFDSKGEPVLSGCCEIFHFDADITKMLDDKFAFAEKARSLGLSVPKSFKITDPEQVLNFDFSQEKRKYILKSIPYDCLRRLNMTKLPCDTFDMTAEFVKSLPISEEKPWIMQEFIPGKEYCTHSTVRDGELRLYCCCESSAFQVNYENVDRPEIRQWVQQFVQEVGLTGEISFDIIQADDGTVYPIECNPRTHSAITMFYNHPGVANAYLNKEPLVEPLQPLADSKPTYWLYHEVWRLTGIRSLKQLQTWIRNILRGKEAIFSVSDPLPFMMVHHWQIPLLLLDNLRRLKGWVRIDFNLGELIESEEY

SEQ ID NO: 82 - protein, 464 aa, Synthetic construct
MAQSISFSSAPATPSVPSTSKIAAIFPNIGTLTLLLLALPINASIVLITLLLRAILRPFQPSAVKAANPKNILISGGKMTKALQLARSFHAAGHRVVLLETHKYWLTGHQYSQAVDKFYTVSAPQENPERYTQALVDIIKQENIDVYIPVTSPLGSYYDSLAKPELSRYCEVFHFDADITQMLDDKYELAQTARSLGLSVPKSFKITSAEQVLNFDFSGETRKYILKSIPYDSVRRLDLTKLPCATPEETAAFVRSLPISPEKPWIMQEFIPGKEFCTHSTVRNGELRLHCCCESSAFQVNYENVENPQILEWVKHFVKELKLTGQISFDFIQAEDGKVYAIECNPRTHSAITTFYDHPKVAEAYLSQEATTETLQPLPTSKPTYWTYHEVWRLTGIRSFKQLKTWIVNIWRGTDAIYKFDDPLPFLMVHHWQIPLLLLKNLRQLKGWTRIDFNIGKLVELGGD

SEQ ID NO: 83 - protein, 435 aa, Synthetic construct
MFQNLGTLVLLAIAFPLNCIVVLTSLLWSFIKQPFNKSIVVNPNSKNILIAGARMTKTLQLARSFHAAGHRVIIIDIEKYWLSGNKYSNSVAGFYTVPDPSKDLEGYVETLHAIANTEKIDFFIPVAIFSVIHYDQGKPPLPDCVEFFHFDADVTKILDDKFAFAETARSFGLSVPKSFKITDPEQVLNFDFSQEKRKYILKSIPYDQVRRLNLTKLPCDTKSETAAFVKSLPISEENPWIMQEFIPGKEYCTHTTARDGESRMYCCCESSAFQVNYENVDQREIMQWASHFTKELGKTGQLSFDFIQAEDGTVYAIECNPRTHSAITMFYNHPGVADAYLGKEPLAESLQPLPDSKPTYWLYHEVWRLNEIRSFKQLQTWVRNIRRGKEAIFEVSDPLPFLMVHHWQIPLLILDNLRRLKGWIRIDFNMGELIE

SEQ ID NO: 84 - protein, 454 aa, Synthetic construct; identical to SEQ ID NO: 47

SEQ ID NO: 85 - protein, 443 aa, Synthetic construct
MKEQIFIVFQNLGTLVLLAIAFPFNCIVVLTSLVWNFIKQPFSQSIVVNPNSKNILIAGARMTKTLQLARSFHAAGHRVIIIDIEKFWSSGNKYSNSVAGFYTVPDPSKDLEGYVESLHAIAKKEKIDFFIPVAIFSVIHYDSQGKPPLPDDVEFFHFDADVTKILDDKFAFAETARSFGLSVPKSFKITDPEQVLNFDFSQEKRKYILKSIPYDQVRRLNLTKLPCDTPSQTAAFVKTLPISEEKPWIMQEFIPGKEYCTHTTARDGESRMYCCCESSAFQVNYENVDQPEIMQWASHFTKELGKTGQLSFDFIQAEDGTVYAIECNPRTHSAITMFYNHPGVADAYLGKEPLAESLQPLSDSKPTYWLYHEVWRLNEIRSFKQLQTWVRNIRRGKEAIFEVSDPLPFLMVHHWQIPLLILDNLRRLKGWIRIDFNMGELID

SEQ ID NO: 86 - protein, 458 aa, Synthetic construct; identical to SEQ ID NO: 51

SEQ ID NO: 87 - protein, 456 aa, Synthetic construct
MIDTVSLNKSLAEKGFGRREIGVIGRNLATLGLLLLVLPINLLLTGVGLISRVSLRNPISQKTILISGGKMTKALLIARRFHAAGHRVILIESHKYWLTGHRFSNAVNKFYTVPAPEKNPSAYIQALLDIIKREKVDLYVPVCSPVASYYDALVKSEMGFLTQVFHCDPEMVKMLDDKFTFAETARKLGLSVPKSFLITHPHQVINFDFQKETRPYILKSIRYDSVRRLDLTKLPCETPEATERFVRSLPISPENPWIMQEFIPGQEYCTHSTVKNGELRMHCTSKSSAFQVNYENIDHPRIQSWVSKFVKELGITGQVSFDFIETEDGEVYAIECNPRTHSAITMFYNHPRVADAYLDEGVWEQPIQPLPDSKPTYWLYHEIWRLTGIRSWKDLQYRWKVLSTGVDAIYSLDDPLPFLMVHHWQIPLLLWQNLLQLRGWVRIDFNIGKLVELGGD

SEQ ID NO: 88 - protein, 445 aa, Synthetic construct
MQKMFAIFQNLGTLTLLAIAFPFNCIVVLSALVWNLISQPFQKQVVFNPDAKNILIGGGRMTKTLQLARSFHAAGHRVILFDIDKNWFSGYRFSNAVAGFYTVPDPIKDLEGYTIALRAIAKQENIDFFVPVGIFANDYFDSKRQPVLSGCCETFHFDADTMKMLDNKFTFTQKARSLSLSVPKAYLITDPEQVLKFDFSNEKNKYILKSIVYDPVFRLDLTKLPMESLEKMAIHVRNLPISKDNPWILQEFITGQEYCTHSTVRNGELTVHCCCESSAFQVNYENVDKPEILQWVSHFVKELQLTGQISFDFIQAEDGTIYAIECNPRTHSAITMYYNHPGLADAYLGQKPLAELLQPLPDSKPTYWLYHEVWRLNEIRSLKQLQTWFKNILRGKDAIFDVNDPLPFLMVHHWHIPLLLLDNLQKLKGWVRIDFNIGKIVQVSD

SEQ ID NO: 89 - protein, 464 aa, Synthetic construct
MDNLFNSSADSSSLSKGWLRSIQGSSLKTLGTLLLLLLMLPFNLALTLTALVWSWVWPFRKRVIASNPKTVMISGGKMTKALQLARSFYMAGHRVILVETHKYWLVGHRYSWAVDRFYTIPDPKQDTEGYLQGLLDIAQKEQVDLYVPVCSPVASYYDALAKELLAQQCDVFHEDAKTVQQLDDKYQFAQAATNLGLTVPKSFKITHPQQVLDFDFSKETHPYIIKSIPYDSVNRLNLTKLPCASRQDTEMFVNSLPISETKPWVMQEFITGQEYCVHSTVKNGELRVYCCCESSAFQVNYEAVDIPEIKQWVTQFVQGMKLTGQMSFDFIRTPTGEVYAIECNPRTHSAITLFYNHPDLAKAYLDPEPFSEPLEPLASARPTYWTYHEFWRLVTHLSSLQEVAYRLGILFKGKDAIFSWNDPLPFLMVHGWQIPLLLLKSLRQGKDWIRIDFNIGKLVQMGGD

SEQ ID NO: 90 - protein, 447 aa, Synthetic construct
MTQIFFVSGRGSAVLQNLGTLVLLLFLLPFNLIAVAFSAVINIFSGSKQRLTKTDVPKRILITGAKMTKALQLARSFHQRGHEVYLVETHKYWLSGHRFSRAVKGFFTVPTPEKEPDAYCQRLLEIVQQKNIDVFIPVSSPIASYYDSLAKKILEPDCEAIHFDPEITAMLDDKYAFCTKAKELGLSAPKVFCFTSPQQVIDFDFESDGSQYIVKSIPYDSVRRLDLTKLPFEGMESYLRSLPISSEKPWVMQEFIRGQEYCFHATVRKGKIRLHCCSQSSPFQVNYEQVDNPAIYQWVEKFVRELNLTGQICFDMIQTPDGTVYPIECNPRLHSAITMFHDHPGVADAYLLDGEQAITPLPDSKPTYWTYHELWRLLQVRSLSELQAWWHKVSRGTDAILQGDDPLPFLMLHNWQIPLLLLDNLRRLKGWIRIDFNIGKLVELEGD

SEQ ID NO: 91 - protein, 464 aa, Synthetic construct
MTQSISLSLPESTTPSTGIKVKIVALFKTLGTLTLLLIALPFNVLIVLISLLWGIVRVPFTKNVVATHPQTILVSGAKMTKALQLARSFHADGHRVILIEGHKYWLSGHRFSKAVSRFYTVPAPQSDPEGYIQALIEIVKKEKVDVYVPVCSPVASYYDSLAKPALSEYCEVFHFDADITKMLDDKFAFTEKARSLGLSVPKSFKITDPQQVINFDFSQETRKYILKSINYDSVRRLNLTKLPCDTPEQTAAFVKSLPISPETPWIMQEFIPGKEFCTHSTVRDGELRLHCCCHSSAFQINYENVENPQIQAWIQHFVKSLRLTGQVSFDFIQAEDGQVYAIECNPRTHSAITMFYNHPGVAEAYFGKTPLAAPLEPLPSSKPTYWTYHEIWRLTGVRSWKQLQTRLNILLRGTDAIYCLDDPIPFLTLHHWQIPLLLLQNLQQLKAWVKIDFNIGKLVELGGD

SEQ ID NO: 92 - protein, 462 aa, Synthetic construct
MAQSISLSVPKSTTPSTGVSIKIVALFKTLGTLTLLLIALPINAFIVLLSLLWGILFTKKPAVAAHPQNILVSGGKMTKALQLARSFHAAGHRVILIEGHKYWLSGHRFSNAVSRFYTVPAPQDDPQGYTQALLEIVKQEKIDIYVPVCSPVASYYDSLAKPALSEYCEVFHFDADITKMLDDKFAFTDQARSLGLSVPKSFKITDPEQVINFDFSKETRKYILKSISYDSVRRLNLTKLPCDTPEETAAFVNSLPISPEKPWIMQEFIPGKELCTHSTVRDGELRLHCCSDSSAFQINYENVENPQIREWVQHFVKSLGLTGQVSFDFIQAEDGTAYAIECNPRTHSAITMFYNHPGVAEAYFGKTPLAAPLEPLADSKPTYWVYHEIWRLTGIRSGKQLQTWFARLVRGTDAIYKIDDPLPFLTLHHWQIALLLLQNLQQLKGWVKIDFNIGKLVELGGD

SEQ ID NO: 93 - protein, 442 aa, Synthetic construct; identical to SEQ ID NO: 42

SEQ ID NO: 94 - protein, 464 aa, Synthetic construct
MAQSISVSSSPAIPSFPSETKIAVIIQNLLTLALLLLALPINAAIVLVTLLWHTISRPFQQPATKAANPKNILISGGKMTKALQLARSCAAAGHRVILIETHKYWLSGHRFSQAVDKFYTVPAPQENPERYTQALIDIIKQENIDVYIPVTSPLGSYYDSLAKPLLSEYCEVFHFDIDITEKLDDKFAFAETARSLGLSVPKSFKITSAEQVLNFDFSQESRKYILKSIPYDSVRRLDLTKLPCATPEETAAFVRSLPISPDKPWIMQEFIPGKEFCTHSTVRDGELRLHCCCESSAFQVNYENVENSQIREWVRHFVKELKLTGQVSFDFIQAEDGRVYAIECNPRTHSAITTFYDHPQVAQAYLDNEPMAETLQPLPSSKPTYWTYHEVWRLTGIRSFTQLKKWIANIWRGTDAIYKPDDPLPFLMVHHWQIPLLLLKNLRQIKGWTRIDFNIGKLVELGGD

SEQ ID NO: 95 - protein, 459 aa, Synthetic construct
MTQSISVASVGQTTQSVTLGLRISALFKNLATLALLLLVLPINAAIVLVSLLLGSQSQAIATEPKNILISGGKMTKALQLARSFHAAGHRVVLVETHKYWLTGHRFSKAVSRFYTLPTPQSDPEAYTQALLDIVQKESINVYVPVCSPVSSYYDSLAKPVLSKYCEVFHCDADVTQMLDDKYAFAEKARSLGLSVPKSFKITDPKQVINFDFSQEKRKYILKSIPYDSVRRLDLTKLPCESPEATADFVNSLPISSQKPWIMQEFIPGKEFCTHSTVRNGELRMHCCCESSAFQVNYENVDHPQILEWVRHFVKALGITGQVSFDFIEAQDGTIYAIECNPRTHSAITMFYNHPDVANAYLSEIPQVEPIQPLINSKPTYWTYHEIWRLTGIRSFSQLQTWVKNFFGGKDAIYSLSDPLPFLTVHHWQIPLLLLQNLQQLKGWIRIDFNIGKLVEFGGD

SEQ ID NO: 96 - protein, 463 aa, Synthetic construct; identical to SEQ ID NO: 55

SEQ ID NO: 97 - protein, 465 aa, Synthetic construct
MTQSISFSSPVPATPPFCVKTRFIALFQNLGALTLLLLALPINVAIVLISLIWSFLSRLFSTQETTVAGAKNILISGGKMTKALQLARFFSAAGHRVVLIETHKYWLSGHRFSNAVSRFYTTPTPQDEPEEYIQTLVDIVKRENIDVYVPVTSPVASYYDSLAKPALSPYCEVLHFDADVTKMLDDKFAFSEKARALGLSVPKSFKITNPEQVLNFDFSQETRKYILKSLPYDSVRRLDLTKLPCNTPEETAAFVKSLPISLEKPWIMQEFIPGKEFCTHSTVRNGDLKLHCCSESSAFQVNYENVKNPKIQEWVRHFVKGLGLTGQVSFDFIQADDGKVYAIECNPRTHSAITMFYNHPQVADAYLGTEPLAEPLAPVPNSKPTYWLYHEVWRLTGIRSFAQLQSWIRNILRGTDAIYELHDPLPFLMVHHWQIALLLLNNLRQLKGWTKIDFNIGKLVELGGD

SEQ ID NO: 98 - protein, 442 aa, Synthetic construct
MRKHIFVVFQNLGTLVLLALAFPLNSIVVLTSLLWNFLKQPFSKSIVVNPNSKNILIAGARMTKTLQLARSFHAAGHRVIIIDIEKFWSSGNKYSNSVAGFYTVPDPSKDLEGYVETLHAIAKTEKIDFFIPVAIFSVIHYDRGKPPLPDFCEFFHFDADVTKSLDDKFAFAETARSFGLSVPKSFKITNPEQVLNFDFSQEKRKYILKSIPYDQIRRLNLTKLPCDTQSETAAFVKSLPISEENPWIMQEFIPGKEYCTHTTARDGESRMYCCCESSAFQVNYENVDRLEIMEWASHFTKQLGKTGQLSFDFIQAEDGTVYAIECNPRTHSAITMFYNHPGVADAYLGKNPLAESLQPLGDSKPTYWLYHEVWRLNEIRSFKQLQTWLRNIRRGKEAMFEVSDPLPFLMVHHWQIPLLILDNLRRLKGWIRIDFNMGELIE

SEQ ID NO: 99 - protein, 449 aa, Synthetic construct
MVELQFIKARIFAVFRNLGTLALLAIAFPFNCIVVLAALLWNFFTRPFQKQVVLSENPKNILIGGGRMTKTLQLARSFHAAGHRVILVDIHKYWLSGHRFSKAVAGYYTVPEPQKDLEGYTQALRAIAKKENIDFFIPVAIFAVSYFDPQNKPVLAGCCEIFHFDGEVTKMLDDKFAFAEKARSFGLSVPKSFKITAPEQVLNFDFSQEKNKYILKSIPYDSVRRLNMTKLPCDTTEQTAAFVKSLPISEENPWIMQEFIPGQEYCTHSSLRNGELRLHCCCESSAFQVNYENVDKPEIMQWVSHFVKELGLTGEASFDIIQAVDGTVYPIECNPRTHSAITMFYNHPGVADAYLGKEPLAEPLQPLPDSKPTHWLYHEVWRLTGIRSLKQLQTWVRNILRGKDAIFEVHDPLPFLMVHHWQIPLLLLDNLRRLKGWIRIDFNLGELIE

SEQ ID NO: 100 - protein, 472 aa, Synthetic construct
MHFNCGAEKLMAQSISLSLPKSTTPSTGVRIKIVALFKTLGTLTLLLIALPINAFIVLLSLLWSIPFTKKPAVAAHPQNILVSGGKMTKALQLARSFHAAGHRVILVEGHKYWLSGHRFSKAVSRFYTVPAPQDDPEGYTQALLEIVKQEKIDIYVPVCSPIASYYDSLAKPALSEYCEVFHFDADITKMLDDKFAFTDQARSLGLSVPKSFKITDPEQVINFDFSKETRKYILKSISYDSVRRLNLTKLPCDTPEETAAFVNSLPISPEKPWIMQEFIPGKELCTHSTVRDGELRLHCCSDSSAFQINYENVENPQIREWVQHFVKSLGLTGQVSFDFIQAEDGTAYAIECNPRTHSAITMFYNHPSVAEAYFGKTPLAAPLEPLADSKPTYWVYHEIWRLTGIRSGKQLQTWFTRLVRGTDAIYKIDDPLPFLTLHHWQIALLLLQNLQQLKGWVKIDFNIGKLVELGGD

SEQ ID NO: 101 - protein, 423 aa, Synthetic construct
MFPINLTLVITAFLTNLITLPFQKKITYENPKNILLTGGKMTKSLQLARSFHRAGHKVFMVETHKYWLSGHQYSKAVKKFLTVPAPEKDPEGYCQSLLDIVKREKIDVFIPVSSPVASYYDSLAKPILSPYCEVFHFDTEMTKTLDDKFSLCEQARVLGLTAPKVFLITSPGEIINFDFSQEQNPYIIKSIQYDSVTRLDMTKFPFEGMKEYVKKLPISKERPWVMQEFIKGQEYCTHSTVRDGEIRLHCCSKSSPFQVNYEQVDNPEIFQWVQKFVKELNLTGQISFDFMQTEDGKVYPIECNPRTHTAITMFYDHPGLADAYLEPGKNQPHIEPLPTSKPTYWLYHELWRITGIRSFNDLTNWLNKVIKGKDAMLDKDDPLPFLMVHHWQIVLLLLQNMVKLKGWVRIDFNIGKLVEIGGD

SEQ ID NO: 102 - protein, 458 aa, Synthetic construct; identical to SEQ ID NO: 51

SEQ ID NO: 103 - protein, 465 aa, Synthetic construct
MAQSISLSLGNSPTSSTGVWVKLVALFKTLGTLTLLLIALPFNALIVLISLLWGFVRSPFRQKAVVAEHPQTILVSGAKMTKALQLARCFHAAGHRVILIEGHKYWLSGHRFSKAVSGFYTVPAPQLDPEAYIQALVDIVEKEQVDVYVPVCSPVASYYDSLAKPALSEYCEVFHFDADVTKMLDDKFAFTAQARSLGLSVPKSFKITDTQQVINFDFSQETHKYILKNIAYDSVRRLNLTKLPCDTPEETAAFVNSLPISEENPWIMQEFIPGKELCTHSTVRDGELRLHCCSDSSAFQINYENVENTQIREWVQHFVKSLALTGQISFDFIQAESGTVYAIECNPRTHSAITMFYNHPGVAEAYLGKTTLDAPLEPLTNSKPTYWIYHEIWRLTGIRSWKQLQTAVNTLLRGTDAIFQLNDPVPFLTLHHWQIPLLLLKNLQQLKGWVKIDFNIGKLVELDGD

SEQ ID NO: 104 - protein, 461 aa, Synthetic construct
MIIHMAQSISLSSPAKTHAPGISASSLKTLGTLTLLLLALPLNASLVLVALLLKSLRPQNFTTEKPKNILISGGKMTKALQLARSFHNAGHRVILLEAHKYWLTGHRFSSAVNKFYTVEAPEKDPEGYIQSLVDIVEKENIDVYVPVCSPVASYYDSLAKKALPQCEVIHCDAEMTQMLDDKHAFAQTAQSFGLSVPKSFKITDPEQVINFDFSQEKRKYILKSIPYDSVRRLDLTRLPCDTPEATAAFVRSLPISSEKPWIMQEFIPGKEYCTHSTVRNGVITLHCCCESSAFQVNYENVDNPKIFEWVSRFVKELGITGQVSFDFIEAEDGNIYAIECNPRTHSAITMFYNHPGVADAYLGTGNNLAEPIQPKFTSKPTYWTYHEIWRLFNTRSWSDFVYRFKIIKHGKDAIFSWQDPLPFLMNPHWQIFLLLIQNLQKNRGWIRIDFNIGKLVELGGD

SEQ ID NO: 105 - protein, 439 aa, Synthetic construct
MGNGALAENLKEDDKTVIWRPHEEKYRTSEWYTGSGQITTADEGLSFEVTAVYQLKSEVKVVKDIFAISNHTLANIYRPRSRCIAVVDQTVAELYGEKIEGYFQAQEIPLELMVIRAWESDKTPETVHRILAFLGKDGCDVSRNEPVLVIGGGVLSDVAGLACALQHRRTPYIMIGTTIIAAIDAGPSPRTCTNGTQFKNSIGVYHPPVLTLVDRQFFSTLDMGHIRNGMAEIIKMAVTDDKELFELLEQYGQELIKTRFATIDASEELEKIADLIIYKALYAYMKHEGTNMFETYQDRPHAYGHTWSPRFEPAVKLMHGHAVTIGMAFGATLAQELGWLSQEECQRIINLSSKLGLSVFHPILEDVQIMVDGQKNMRRKRGDGGLWAPLPTTIGACDYVQEVEPELLNQAVVAHKKYCSQLPHEGAGEQMYLSDLGLE

SEQ ID NO: 106 - protein, 403 aa, Synthetic construct
MSNLQAQVVAGDRSFRVEGYERIEYDLIYVDGVFAIENTELADSYRPYGRALMVVDEAVHDIYGDRISAYFDHHEIALTVVPVHIAETAKSLETFERIVGEFDAFGLVRTEPVLVVGGGLTTDVAGFACASYRRNTPYIRIPTTLIGLIDASVSIKVAVNYGKHKNRLGAYHASQKVLLDFSFLGTLPEDQVRNGMAELIKISVVGNLEIFEMLEQYGPELLRTRFGHLDGTAELRSVADKLTYSAIATMLELEAPNLHEIDLDRVIAFGHTWSPTLELTPPAPFFHGHAINIDMALSTTVAEQRGHLSTADRDRVLGVMSSIGLALDSPYLTPELLSEATASILKTRDGILRAAVPDPIGTCRFLNDLDAAELADVLTLHKKICLDFPRAGEGLDMFTAPTP

SEQ ID NO: 107 - protein, 410 aa, Synthetic construct
MAGIKATFTSTDCAFHIQGYEKIDFSLLYVNGAFKIGNPEIAESYAPFRRCLMVIDQTVYGLYRQQIDQYFAHYQIDLTVFQVSIKEPEKTLRTFEKIVDAFADFGLVRKEPVLVVGGGLTTDVAGFACSAYRRKTNYIRVPTSLIGLIDASVAIKVAVNHGKLKNRLGAYHASQKVILDFSFLGTLPIDQIRNGMAELIKIAVVGNQEIFELLEEHGAALLHSRFGYLNGTPELQAVGHRLTYKAIQAMLELEVPNLHELDLDRVIAYGHTWSPTLELTPEPPMLHGHSVNIDMAFTATIAQLRGYISVEDRNRILGLMSRLGLAIDSPYLTPELLWKATEAITRTRDGLQRAAAPRPIGQCVFMNDLTRSELDKALAVHRAIAQNYPRQGNGEDMYVRLEPALEGAGV

SEQ ID NO: 108 - protein, 409 aa, Synthetic construct
MSSVQAKVEVTDQSFHLEGYEKIEFNLDLIEGLFEVGNSGLADNYRTLGRCLAVVDHNVDRLYGDQLRSYFEYYEIDLTVFAIEITEPTKTIDTFLKITDAFCDFNLKRKEPVLVIGGGLVLDVAGFACSAYRRSTNYIRVPSTLIGLIDAGVAIKVAVNHGKLKNRLGAYHPPKQVILDFSFLKTLPVDQIRNGMAELVKIAVVSNEEVFNLLEQHGEELLYNHFGFVGNDAELKQIGHRVNYESIKTMLELEAPNLHELMLDRVIAYGHTWSPTLELAPQIPLLHGHAVNIDMAISATIAEKRGYISALDRDRILGLMSRLGLALDHPLMEIDLMWKATQSIMLTRDGFLRAAMPRPIGTCYFVNDLTREELESAIADHKRLCADYPRAGAGIDAYVGSSELIGSAN

SEQ ID NO: 109 - protein, 415 aa, Synthetic construct
MSNPQAVLSATDTEFRVESWERIEFTLSYVDGVFAPHNTELADLYRPWGRCLMVIDETVHEHYGDPIRSYFDHHDIAVTLVPLTIAETAKSLRTLERIVDAYADFGLLRTEPVLVVGGGLTTDVTGFACASYKRGTPYVRIPTTLIGLIDASVAMKVAVNHGRHKNRLGAFHASQQVLLDFSFLATLPEAQVRNGVAEMIKIATVANAGLFDLLEKYGDDLLATRFGHREGTPELRQIAHRCTYDAIHTMLELEHRNLHELDLDRVIAFGHTWSPTLELAPPTPMLHGHAIAIDMAFSATLAARRGDITTGERDRIHRLFSGLGLSVDSTYLTEQLLIDATASIMQTRAGKLRAALPRPIGTCHFANDIEHTELIETLAAHKAVVAGLPTSVEGVEMWSSAKTELTTAPNTEART

SEQ ID NO: 110 - protein, 406 aa, Synthetic construct
MTTNLTATVTATENDFRVRAVEERDYLLTYVDGAFSPESSRIADHHRAHGRCLMIVDANVHRLHGDRIRAYFEHHGIALTALPLAIDETQKSLRTVERIVDAFGEFGLIRKEPVLVVGGGLLTDVAGFACAVFRRSTDYVRVPTSLIGLIDASVAIKVAVNHGRTKNRLGAFHASKEVVLDFSFLGTLPTEQVRNGMAELVKIAVVANAEVFRLLEKYGEDLLHTAFGTVDGTPQLRETARKVTHEAIGTMLALEAPNLRELDLDRAIAFGHTWSPALELAPETPYLHGHAISVDMALSCTIAERRGYLATSERDRIFWLLSKVGLSLDSPHLTPELLRAATESIVQTRDGLQRAAMPRPIGTCCFVNDLTESELLDGLAAHRELVARYPRGGAGEDVRVTRSGAA

SEQ ID NO: 111 - protein, 414 aa, Synthetic construct
MSTVQAKFEATETAFHVEGYEKIDFSLVFVNGAFDTKNRELADSYRNFGRCLAVVDANVNRLYGSQICEYFKYYNIDLNLFPVTISEPTKNLDTFQSIVDAFADFGLVRKEPVLIVGGGLVTDVAGFACAAYRRSTNYIRIPTTLISLVDAGIAIKVAVNHGKLKNRLGAYHAPKKVMLDFSFLRTLPTPEVRNGMAELVKIAVVSNVEVFELLCEYGADLLTTHFGFDGGTPLLKEVAHRINYESIKTMLALETPNLHELDLDRVIAYGHTWSPTLELAPSVPLLHGHAVNIDMALSATIAEKRGYITVEERDRILGLMSQLGLALDHPLLDIDLLWSATQSITLTRDGLQRAAMPRPIGKCFFVNDLTREELDAALAEHKHACAQYPRAGAGVDAYVGSYQQQNLIEGIANV

SEQ ID NO: 112 - protein, 278 aa, Synthetic construct
MSTTIAKPTARPVTPVGILAKKLEAIVQKINQRTDLPADLVDNITQAWQLAAGLDPYLEEYTTSESSALTALAEKTSTEAWQEHFSEGTTVRPLEQEMLSGHVEGQTLKMFVHMTKAKRVLEIGMFTGYSALAMAEALPPDGVLVACEVDPFAAEVGQAAFDKSPDGKKIRVELGPALETLNKLVEAGESFDMVFIDADKKEYITYFQTLLDTNLLAPSGFICVDNTLLQGEVYLPTQQRTANGEAIAQFNRAVALDPRVEQVILPLRDGLTIIRRTA

(19) United States
(12) Patent Application Publication
(10) Pub. No.: US 2026/0272801 A1
(43) Pub. Date: Sep. 17, 2026

(54) BIOCATALYTIC SYNTHESIS OF UNNATURAL MYCOSPORINE-LIKE AMINO ACIDS

(71) Applicant: University of Florida Research Foundation, Incorporated, Gainesville, FL (US)

(72) Inventors: Yousong Ding, Gainesville, FL (US); Manyun Chen, Gainesville, FL (US)

(73) Assignee: University of Florida Research Foundation, Incorporated, Gainesville, FL (US)

(21) Appl. No.: 18/869,598

(22) PCT Filed: May 26, 2023
(86) PCT No.: PCT/US2023/023710

§ 371 Date: Nov. 26, 2024

Related U.S. Application Data
(60) Provisional application No. 63/346,791, filed on May 27, 2022.
(60) Provisional application No. 63/392,462, filed on Jul. 26, 2022.

(51) Int. Cl. A61K 8/44; A61K 8/35; A61Q 17/04; C12N 9/00; C12P 13/00

(52) U.S. Cl. CPC A61K 8/44; A61K 8/35; A61Q 17/04; C12N 9/93; C12P 13/005; C12Y 603/02004

(57) ABSTRACT

The present invention relates to methods of producing compounds of interest in a recombinant microorganism. In particular, the present invention relates to using a recombinant microorganism comprising a heterologous nucleic acid encoding one or more mycosporine-like amino acid (MAA) biosynthetic enzymes (e.g., MysD) to produce compounds of interest using unnatural amino acids or amine containing substrates. Compositions comprising compounds produced using such methods are also provided herein. The present disclosure also provides methods of preventing sunburn, cancer, and chronic inflammatory diseases by administering such compositions to subjects in need thereof.

### CROSS-REFERENCE TO RELATED APPLICATIONS

This application claims priority to U.S. Provisional Application No. 63/346,791, filed May 27, 2022, and U.S. Provisional Application No. 63/392,462, filed Jul. 26, 2022, all of which are incorporated herein by reference in their entireties.

### GOVERNMENT SUPPORT

This invention was made with government support under Grant No. 2108383 awarded by the National Science Foundation. The government has certain rights in the invention.

### BACKGROUND OF THE INVENTION

Skin cancers are among the most common cancer types in the United States with about 1.2 million Americans living with melanoma and 3 million more affected by nonmelanoma skin cancers.^{1,2} Solar radiation, especially ultraviolet (UV) radiation, is an established risk factor of skin cancers,³ as more than 90% of melanoma in some populations are linked to sunlight exposure.⁴ UV rays, mainly UVA (315-400 nm) and UVB (280-315 nm), induce a variety of damages on biomolecules (e.g., DNA and proteins) of living organisms on earth.⁵ In addition to behavioral changes, proper skin protection from excessive sun exposure has proven to be effective in reducing skin cancers.⁶ In this regard, many organic and inorganic compounds have been developed to dissipate the energy of UV rays and/or directly block their reach on the skin, and some have been used as active ingredients of commercial sunscreens.⁷ However, there are increasing concerns regarding the potential negative health impact of synthetic sunscreens (e.g., endocrine disruption, neurotoxicity, and systemic absorption),⁸⁻¹⁰ while multiple organic UV filters are accumulated in almost all water sources globally and may be potential contributors to coral reef bleaching, raising a severe environmental concern over their use.¹¹ Accordingly, there is a need for safer, biodegradable, and environmentally friendly new compounds with UV-modulating, anti-inflammatory, and/or anti-oxidative properties.

### SUMMARY OF THE INVENTION

Natural organisms have developed multiple effective UV mitigation strategies when utilizing solar energy, including the biosynthesis of diverse natural products as photoprotectants.^{12,13} These natural products (e.g., flavonoids, phenols, terpenoids, and polyketides) absorb UV radiation and release energy through thermal de-excitation, similar to synthetic chemical UV filters, while providing additional protection from UV-induced damages with other biological functions, e.g., antioxidants, anti-inflammation, and immunomodulation.¹⁴ These compounds provide important inspiration for the development of new generation sunscreens.¹⁵ One such example, mycosporine-like amino acids (MAAs), are a family of natural, thermally and photochemically stable UV protectants (FIG. 1A).¹⁶ The superior UV protection properties of MAAs has potential to impact the development of next-generation sunscreens for broad cosmetic applications if the low quantity available from natural resources or the lack of efficient synthetic preparation were properly addressed.²⁵⁻²⁶

Accordingly, in one aspect, the present disclosure provides methods for producing a compound (e.g., an MAA, or a derivative thereof, and any of the compounds delineated herein). The methods of the present invention comprise culturing a recombinant microorganism under conditions suitable for production of the compound and isolating the compound from the recombinant microorganism, wherein the recombinant microorganism comprises a heterologous nucleic acid encoding one or more mycosporine-like amino acid (MAA) biosynthetic enzymes (e.g., a D-alanine-D-alanine ligase (MysD), or a homolog thereof). In some embodiments, the one or more MAA biosynthetic enzymes include MysA, MysB, MysC, MysE, and/or MysH.

In certain embodiments, the compound is of Formula (I), or a salt thereof:

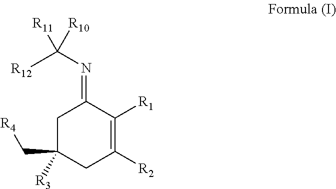

wherein R₁, R₂, R₃, R₄, R₁₀, R₁₁, and R₁₂ are as defined herein.

In some embodiments, the methods described herein further comprise providing a substrate of the one or more MAA biosynthetic enzymes to the recombinant microorganism. In certain embodiments, the substrate is a compound of Formula (II), or a salt thereof:

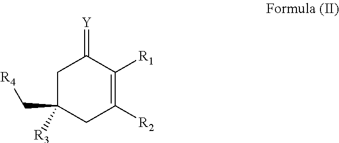

wherein R₁, R₂, R₃, R₄, and Y are as defined herein.

In another aspect, the present disclosure provides a recombinant microorganism comprising a heterologous nucleic acid encoding one or more MAA biosynthetic enzymes. In some embodiments, the one or more MAA biosynthetic enzymes comprise a D-alanine-D-alanine ligase (MysD), or a homolog thereof.

In another aspect, the present disclosure provides compositions comprising a compound produced by the methods disclosed herein. In some embodiments, the composition comprises an excipient. The composition may be formulated for topical administration (e.g., for use as a sunscreen or a cosmetic). In certain embodiments, the present disclosure provides methods of making the compositions disclosed herein. Such methods may comprise producing a compound using the methods disclosed herein and adding the compound to one or more excipients to produce the composition.

In another aspect, the present disclosure provides methods of administering a composition (e.g., any of the compositions described herein), comprising applying to composition to a subject. In some embodiments, the composition is applied to the skin of a subject. In certain embodiments, the method is a method of preventing sunburn. In certain embodiments, the method is a method of preventing cancer. In certain embodiments, the method is a method of preventing or treating a chronic inflammatory disease.

In another aspect, the present disclosure provides compounds produced using the methods disclosed herein. In some embodiments, the compounds are of Formula (I), or a salt thereof, as provided herein.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1B show the structures and biosynthesis of mycosporine-like amino acids. FIG. 1A provides the chemical structures and maximal absorbance of representative mycosporine-like amino acid analogs. FIG. 1B shows the biosynthetic pathway of shinorine, porphyra-334, palythine-Ser, and palythine-Thr.

FIGS. 2A-2B show structures of the native and unnatural substrates. FIG. 2A provides the chemical structures of the substrates tested in the MysD reaction. FIG. 2B shows the chemical structures of the corresponding predicted MAA analogs.

FIG. 3 shows extracted ion chromatogram (EIC) traces of the in-vitro MysD reaction using different amino acid substrates. Five MAA analogs were detected.

FIG. 4 shows HRMS traces of produced unnatural MAA analogs in the MysD reaction. FIG. 4A shows an HRMS trace of the unnatural MAA analog produced from L-ornithine in the MysD reaction. FIG. 4B shows an HRMS trace of the unnatural MAA analog produced from 4-aminobutanoic acid in the MysD reaction. FIG. 4C shows an HRMS trace of the unnatural MAA analog produced from (S)-2-aminobut-3-enoic acid in the MysD reaction, along with its UV absorption spectrum. FIG. 4D shows an HRMS trace of the unnatural MAA analog produced from (S)-2,3-diaminopropanoic acid in the MysD reaction, along with its UV absorption spectrum.

FIG. 5 shows SSN analysis of 249 cyanobacterial MysD homologs with a filter value at 172.

FIG. 6 shows the predicted structure of MysD from Nostoc linckia NIES-25.

FIG. 7 shows unnatural substrates tested in the MysD reaction.

FIG. 8 shows biocatalytic synthesis of unnatural palythines.

FIG. 9 shows the relative activity of MysD mutants compared to WT (L-Thr as the substrate).

FIG. 10 shows the relative activity of MysD mutants compared to WT (α-aminobutyric acid (AABA) as the substrate).

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7th Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds and oligonucleotides described herein, even though only a single tautomeric form may be represented. All such isomeric forms of such compounds herein are expressly included in the present disclosure. The term “isomers” is intended to include diastereoisomers, enantiomers, regioisomers, structural isomers, rotational isomers, tautomers, and the like. For compounds that contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 2 to 20 carbon atoms and one or more carbon-carbon double bonds (e.g., 1, 2, 3, or 4 double bonds) (“C₂₋₂₀ alkenyl”). In some embodiments, the group ═CH₂ along with the sp²-hybridized carbon to which it is attached form an alkenyl group. In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁ alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₂₀ alkenyl. In certain embodiments, the alkenyl group is a substituted C₂₋₂₀ alkenyl. In an alkenyl group, a C═C double bond for which the stereochemistry is not specified (e.g., —CH═CHCH₃ or

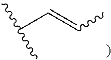

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 11 carbon atoms, at least one double bond, and 1 or more heteroatoms within the parent chain (“heteroC₂₋₁₁ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 carbon atoms, at least one double bond, and 1 heteroatom within the parent chain (“heteroC₂ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₂₀ alkenyl. In certain embodiments, the heteroalkenyl group is a substituted heteroC₂₋₂₀ alkenyl.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₁₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 carbon atoms, at least one triple bond, and 1 heteroatom within the parent chain (“heteroC₂ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₂₀ alkynyl. In certain embodiments, the heteroalkynyl group is a substituted heteroC₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R_{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R_{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R_{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion; each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃, —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S)N(R^{bb})₂, —SC(═O)SR^{aa}, —SC(═O)OR^{aa}, —SC(═O)N(R^{bb})₂, and —SC(═O)R^{aa}, wherein R^{aa} and R_{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(—NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphino” refers to the group —P(R^{cc})₂, wherein R^{cc} is as defined herein.

The term “phosphono” refers to the group —(P═O)(OR^{cc})₂, wherein R^{aa} and R^{cc} are as defined herein.

The term “phosphoramido” refers to the group —O(P═O)(N(R^{bb})₂)₂, wherein each R^{bb} is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R_{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂RC, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R_{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3 -phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)- 6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl (o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl (pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R_{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5 -dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3″′-[N-(imidazolylmethyl)]trityl Ether (IDTr-OR), 4,4′-Dimethoxy-3″′-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio) pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O) (NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In some embodiments, the subject is at risk of developing a disease or condition due to environmental factors (e.g., exposure to the sun).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient to provide anti-oxidative or anti-inflammatory effects. In some embodiments, a therapeutically effective amount is an amount sufficient to provide UV-modulating effects (e.g., absorption of UV wavelengths between 280 and 400 nm). In certain embodiments, a therapeutically effective amount is an amount sufficient for preventing sunburn. In certain embodiments, a therapeutically effective amount is an amount sufficient for preventing cancer. In certain embodiments, a therapeutically effective amount is an amount sufficient for preventing or treating a chronic inflammatory disease.

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, cancer is skin cancer (e.g., basal-cell skin cancer, squamous-cell skin cancer, or melanoma).

The terms “inflammatory disease” and “inflammatory condition” are used interchangeably herein, and refer to a disease or condition caused by, resulting from, or resulting in inflammation. A “chronic inflammatory disease” is an inflammatory disease that causes symptoms over a prolonged period of time. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent). Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenia purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

A “microorganism” refers to a single-celled organism, or a colony of such cells. In some embodiments, the microorganism is a eukaryote. In certain embodiments, the eukaryote is a species of yeast. In some embodiments, the microorganism is a prokaryote. In certain embodiments, the prokaryote is a species of cyanobacteria or a species of bacteria from the human microbiome. In certain embodiments, the prokaryote is E. coli. A “recombinant microorganism” refers to a microorganism that has been genetically altered to express one or more heterologous genes. The genome of the microorganism may be altered, for example, by genetic engineering techniques. In some embodiments, the microorganism is transformed with a vector comprising one or more heterologous genes (e.g., heterologous nucleic acid encoding one or more MAA biosynthetic enzymes, as described herein).

The term “cyanobacteria” refers to members from the group of photoautotrophic prokaryotic microorganisms which can utilize solar energy and fix carbon dioxide. Cyanobacteria are also referred to as blue-green algae. The cyanobacteria species of the present invention can be selected from the group consisting of Synechocystis, Synechococcus, Anabaena, Chroococcidiopsis, Cyanothece, Lyngbya, Phormidium, Nostoc, Spirulina, Arthrospira, Trichodesmium, Leptolyngbya, Plectonema, Myxosarcina, Pleurocapsa, Oscillatoria, Pseudanabaena, Cyanobacterium, Geitlerinema, Euhalothece, Calothrix, and Scytonema.

The term “human microbiome” refers to the aggregate of all the microorganisms that reside on or within human tissues. In some cases, the human microbiome refers specifically to all of the species of bacteria that reside on or within human tissues. Species of human microbiome bacteria for use in the present invention can be selected from the group consisting of, but not limited to, Achromobacter, Acidaminococcus, Acinetobacter, Actinomyces, Aeromonas, Aggregatibacter, Acidaminococcus, Anaerobiospirillum, Alcaligenes, Arachnia, Bacillus, Bacteroides, Bacterionema, Burkholderia, Bifidobacterium, Buchnera, Butyriviberio, Campylobacter, Capnocytophaga, Candida, Clostridium, Chlamydia, Chlamydophila, Citrobacter, Cornybacterium, Cutibacterium, Demodex, Eikenella, Epidermophyton, Enterobacter, Enterococcus, Escherichia, Eubacterium, Faecalibacterium, Flavobacterium, Fusobacterium, Gingiva, Gordonia, Haemophilus, Lactobacillus, Leptotrichia, Malassezia, Methanobrevibacter, Morganella, Mycoplasma, Microbacterium, Micrococcus, Moraxella, Mycobacterium, Mycoplasma, Neisseria, Peptococcus, Peptostreptococcus, Plesiomonas, Porphyromonas, Propionibacterium, Providencia, Pseudomonas, Ruminococcus, Rothia, Ruminococcus, Sarcina, Staphylococcus, Streptococcus, Torulopsis, Treponema, Trichophyton, Veillonella, Vibrio, Wolinella, and Yersinia.

The term “unnatural amino acid” refers to amino acids that are non-proteinogenic amino acids, i.e., amino acids that are not L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine, and L-valine. In some embodiments, the unnatural amino acid is an unnatural α-amino acid. In some embodiments, the unnatural amino acid is an unnatural β-amino acid. In some embodiments, the unnatural amino acid is an unnatural γ-amino acid.

Another object of the present disclosure is the use of a compound as described herein in the manufacture of a medicament for use in the treatment of a disorder or disease described herein. Another object of the present disclosure is the use of a compound as described herein for use in the treatment of a disorder or disease described herein. Another object of the present disclosure is the use of a compound as described herein in the manufacture of a sunscreen for use in skin protection described herein. Another object of the present disclosure is the use of a compound as described herein for use in skin protection described herein. Another object of the present disclosure is the use of a compound as described herein in the manufacture of a sunscreen for use as a cosmetic as described herein. Another object of the present disclosure is the use of a compound as described herein for use as a cosmetic as described herein.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Methods for Producing a Compound

In one aspect, provided herein are methods for producing a compound comprising a) culturing a recombinant microorganism under conditions suitable for production of the compound; and b) isolating the compound from the recombinant microorganism. In some embodiments, the recombinant microorganism comprises a heterologous nucleic acid encoding (e.g., that encodes) one or more mycosporine-like amino acid (MAA) biosynthetic enzymes, wherein the one or more MAA biosynthetic enzymes comprise a D-alanine-D-alanine ligase (MysD), or a homolog thereof.

Exemplary MysD enzymes for use in the present invention include, but are not limited to, the amino acid sequence of SEQ ID NO: 1, or an amino acid sequence at least 70%, at least 75%, at least 80%, at least 85%, at least 90%, at least 95%, or at least 99% identical to the amino acid sequence of SEQ ID NO: 1:

A0A1Z4LFR3
(SEQ ID NO: 1)
MPVLRILHLVGSAQDDFYCDLSRLYAQDCLAAMAELPYDSAIAYITPDGQ
WRFPRSLSREDIAQAKPMPVSEAIEFIAAQNIDIVLPQMFCIPGMTYYRA
LFDLLEIPYIGNTPDLMAITAHKARTKAIVEAAGVKVPRGEVLRRGDVPT
ITPPVVIKPVSSDNSLGVTLVKDAAEYEAALEKAFEHGDEAIVETFIEGR
EVRCGIIVKDGELIGLPLEEYLIDSQEKPIRTYADKLKKTDDGSLGFAAK
GNNKSWILDPNDPITQKVQEVAKKCHQALGCRHYSLFDFRIDSQGQPWFL
EAGLYCSFAPKSVISSMAKAVGIPLNELLTIAIAETLGSNKYSDRISVVE
INEPSKTPRKERELSQMI

Biosynthetic enzymes other than MysD may also be encoded by the recombinant microorganism used in the methods disclosed herein. In some embodiments, the one or more MAA biosynthetic enzymes further comprise a phytanoyl-CoA dioxygenase (MysH), or a homolog thereof. Exemplary MysH enzymes for use in the present invention include, but are not limited to, those of SEQ ID NOs: 2-12, or an amino acid sequence at least 70%, at least 75%, at least 80%, at least 85%, at least 90%, at least 95%, or at least 99% identical to the amino acid sequence of any one of SEQ ID NOs: 2-12:

A0A1Z4LFF0 (SEQ ID NO: 2)
MASLENQIILITGASSGIGTACAKIFAGAGAKLILAARRLERLQQLADIL
TQDFNTEVHLLELDVRDRSAVESAISNLPASWSDIDILINNAGLSRGLDK
LHEGSFTDWEEMIDTNIKGLLYLSRYVVPGMVSRGRGHVVNLGSIAGHQT
YPGGNVYCATKAAVRAISEGLKQDLLGTPVRVTSVDPGMVETEFSQVRFH
GNAQRANQVYQGVTPLTPDDVADVIFFCVTRSPHVNINEVVLMPVDQASA
TLVNRRT
A0A367QPY5 (SEQ ID NO: 3):
MLKVDTLKISSQQVEAFERDGVICVKNALDDIWVERLRTAVDRNISIPGP
LEEKNAPRPEGSVEHASSLWLVDADFRALAFESPLPTLAAQVLKSEKLNF
LADGFFVKKPKTNGHIGWHNDLPYWPVQGWQCCKIWLPLDTVKQENGRLE
YIKGSHQWGKELRERSNPSWFVEPEPHEILSWDMEAGDCLIHHFLTIHHS
VTNISSTQRRAIVTNWTGDDVTYYQRPKAWPFKPLEEIDLPEFNSFKTKK
VGEPIDCDIFPRVEVFR
A0A2Z6D3B5 (SEQ ID NO: 4):
MLKLELPKITLQEIEAFEQDGVICVKNVLDNIWVERMRKAVDKNISIAGP
LEVKGISKPEGNVEHTNSLWLVDADFRALVFESPLATLAAQILKSTKLNF
LADGFFVKQPKATSRVGWHNDLPYWPIQGWQCCKIWLALDKVNQQNGRLE
YIKGSHRWGKELREDSNPAWFSQPESHELLSWDMEPGDCLVHHLLTIHHS
VTNISSTQRRAVVTNWTGDDVTYYPRPKAWPFRPLDEIDIPEFDSLKAKK
PGEPIDCDMFPKIKWHR
A0A2T1LWM2 (SEQ ID NO: 5):
MLIANSSKISRQEVENFKRDGVICLKNVVDDYWVERMRKAVDRNLLNSNG
VRGRKLKTGDVVHDYGLWLKDNDFRDLVFKSPLARVAAQIMESETINFLC
DGFFVKKAKADSHVGWHNDLPYWPVKGWKCCKIWLALDPVNQENGRLEYI
KGSHLWNKDLRENSNVSWFSEPSYSDILYWDMEPGDALVHHFQTIHHSIG
NTTYKSRRAIVTNWTGDDVVYDPSPQTWPFQPIEEIGISEFNSLDTLRSG
ESIDCEIFPKIDLTPSPSPTSRGEQNPNFLKFPHRL
A0A2L2NS52 (SEQ ID NO: 6):
MLKVDTSKITTQQVEAFERDGVICVKNVLDDIWVERMRRAVDKNVLIPGP
LEVKGIPRAEGHVEHTSSLWLTDADFRALAFESPLATLTAQVLKSKKLNF
LGDGFFVKKPKGETGVGWHNDKSYWPIQGWQCCKIWLALDSVNQENGKLE
YIKASHLWGKELREASDPSWFVEPEPHEIISWDMEPGDCLVHHFMTIHHS
VRNTSSTRRRAVVINWTGDDVTYERRPNAWPFRPLEEIDIPEFESLKAKK
SGEPIDCDIFPRVELHR
A0A2C6TQQ8 (SEQ ID NO: 7):
MLKVDTPKISPQQVEAFERDGVICVKNALDDIWIERMRKAVDKNISIPGP
LEGKNTPKKEASAEHTSSLWLVDADFRALAFESPLPKLAVGVLKSEKLNF
LADGFFVKRPEANGRIGWHNDLPYWPVQGWQCCKIWLALDTVKQENGRLE
YIKGSHQWGRELRERSNPSWFVEPEPHEILSWDMEAGDCLIHHFLTIHHS
VTNKSSTQRRAIVTNWTGDDVTYYQRPKAWPFKPLEEIDLPQFNSLKTKK
FGEPIDCDIFPRVEVHRHRTHI
A0A252E419 (SEQ ID NO: 8):
MLKIDTLKISLQQIEAFERDGVICLRNVLDESWVERMRTAVDKNVSIPGP
LEVKGISRPEASVEHTSSLWLVDPDFRALVFESPLSTIAAQLLRSEKLNF
LADGFFVKKPKATSRVGWHNDLPYWPIQGWQFCKIWLALDNVNEENGRLE
YIKGSHQWGKELREDSNPSWFVEPEPHELLSWDMEPGDCLVHHLLTIHHS
VTNISSRQRRAVVTNWTGDDVTYYPRLKAWPFRPLEEIDLPEFNSLKTKK
TGEQIDCYMFPPIQLHR
A0A1Z4LFC6 (SEQ ID NO: 9):
MLKVDTQKISPQQVEAFERDGVICVKNAVDDIWVERMRTAVDKNISIPGP
LEDKNVPKPQGSAEHASSIWLIDADFRALAFESPLPTLAAQVLKSKKLNF
LADGFFVKKPESNGRIGWHNDLPYWPVQGWQCCKIWLALDTVKQENGRLE
YIKGSHQWGKELRERSNPSWFIEPEPHEILSWDMEAGDCLIHHFLTIHHS
VTNISSTQRRAIVTNWTGDDVTYYQRPKAWPFKPLEEIDLPEFNSLKTKK
SGEPIDCDIFPRVQVHR
A0A1Z4IIA4 (SEQ ID NO: 10):
MLKLDLPKITLQEIEAFEQDGVICVKNVLDNIWVERMRKAVDKNLSIAGP
LEVKGITKPEGNVEHSNSLWLVDTDFRALVFESPLANLAAQFLKSTKLNF
LADGFFVKQPKASSRVGWHNDLPYWPIQGWQCCKIWLALDKVNQQNGRLE
YIKGSHRWGKELREDSNPSWFSEPEPHELLSWDMEPGDCLVHHLLTIHHS
VTNISSTKRRAVVTNWTGDDVTHYPRPKAWPFRPLDEIDIPEFDSLKAKK
PGEPIDCDMFPKIKWHR
A0A1Z4HWL1 (SEQ ID NO: 11):
MLKIDTSKISFQQIGAFERDGVICLRNVLDENWVERMRTAVDKNVSINGP
LEAKGISRAEASVEHTSSLWLVDPDFRALVFESPLSTIAAQLLQSEKLNF
LADGFFVKKPKATSRVGWHNDLPYWPIQGWQCCKIWLALDHVNEKNGRLE
YIKGSHKWGKELREDSNPLWFVEPEPHELLSWNMEPGDCLVHHLLTIHHS
VTNISSTQRRAVVTNWTGDDVTYYPRPKAWPFRSVEEIDLPEFNSLKTKK
TGEPIDCDMFPQVQLH
A0A1U71924 (SEQ ID NO: 12):
MLKVDTRKISHQQVEAFERDGVICVKNAVDDIWVQRMRTAVDKNVLIPGP
LEEKNAPKPEASAEHTSNLWLVDADFRALAFESPLPTLAVQVLKSKKLNF
LADGFFVKKPKSNSRIGWHNDLPYWPIQGWQCCKIWLALDTVNQENGRLE
YIKGSHRWGKELRERSNPSWFVEPKPHEILSWDMEAGDCLIHHFLTIHHS
VTNISSRQRRAVVTNWTGDDVTYYQRPKAWPFKSIEEIDLPQFNSFKTKK
SGEPLDCDIFPRIEVHR

In some embodiments, the one or more biosynthetic enzymes comprise an ATP-grasp enzyme (MysC), or a homolog thereof. Exemplary MysC enzymes for use in the present invention include, but are not limited to, the amino acid sequence of any one of SEQ ID NOs: 13-105, or an amino acid sequence at least 70%, at least 75%, at least 80%, at least 85%, at least 90%, at least 95%, or at least 99% identical to the amino acid sequence of any one of SEQ ID NOs: 13-104:

A0A0Q2QHP0
(SEQ ID NO: 13)
MSGVRVHRIWDAGPGRTVAALAALCATLPVDLAVVLVALLVGRQPPRGRLPAEARRTVLLNGGKM
TKALQLARSFHLAGHRVILVESAKYRWTGHRFSRAVDAFYCVPEPGTPGYAPALLNIVRYENVDV
YVPVSSPAGSVPDAVARELLDGACDVVHSDAKTVQLLDDKAEFASTAASLSLQVPDSHRITDARQ
VADFPFPPGRSYILKRIAYNPVGRMNLTRLSAATPDRNAAYARSLSISEDDPWILQEFIEGREYC
THGTARSGRLQVYGCCESSAAQVNYRSVDKPEIRRWVETFVKNLNLSGQVSFDFIEAHDGQVYAI
ECNPRTHSAITMFHDHPDLAAAYLNDGHPLITPKHNSRPTYWIYHELWRLLRHPGRLGRLATILR
GTDAIFTGWDPVPYLMVHHLQIPALLWANLRVGKGWSRIDFNIGKLVENGGD
A0A3S0TU06
(SEQ ID NO: 14)
MGRTLATLVVLFGTLPFDLALVLVALLAGRRPSRGRLPAQARRTILLNGGKMTKALQLARSFHLA
GHRVILVESEKYRWTGHRFSRAVDAFYCVPEPTEPGYALALLDIVRYENVDVYVPVSSPAGSVPD
AVARELLDGACDVVHSDAKTVQLLDDKAEFASTAASLSLRVPDSHRITDARQVVDFAFPAGRSYI
LKRIAYDPVGRMNLTRLSGATPDHNAAYARSLPISEDDPWILQEFIEGREYCTHGTARSGRLQVY
GCCESSSAQVNYRNVDKPEIRRWVETFVKNLNLSGQVSFDFIEARDGQVYAIECNPRTHSAITMF
HDHPDLAAAYLDDNHPLITPNDGARPTYWIYHELWRLLRHRGRISRLVTMLRGKDAIFAGWDPMP
YLMVHHLQIPALLWANLRAGKGWSRIDFNIGKLVENGGD
A0A5A7SAT3
(SEQ ID NO: 15)
MREVFQAKTIGTLALLQVVLPLNLALTTFALLRGVFVAPPPVAVAAQRKTILVSGGKMTKALQLA
RSFHAAGHRVVLVESSKYRFNGHRFSRAVDRFYTVPAPDSDNYAVALLAVVRAEEVDVYVPVCSP
VASYYDALAKDQLSPHCEVLHCDADMVARLDDKYEFFALVASLGLSTPETHRVTAPGQVEEFDFT
GTDYILKSIPYDPVHRRDMTTVPRPTATETTTYARSKPITEATPWIMQEFVRGQEYCTHSLVRDG
AVQVFCCCESSAFQINYRMVDKPEIEEWVGEFAQRLNLTGQVSFDFIQGDDGRLHAIECNPRTHS
AITMFYDHPDLARAYLERGVPVVKPLPHSKPTYWIYHELWRLVTQRGGRAHRLAVIAQGKDAIFD
WDDPLPFLLVHHLQIPSLLLSNLLRRKGWTGIDFNIGKLIEAAGD
A0A0G4HZ53
(SEQ ID NO: 16)
MCRVETRPQVGEHAGMESVPLKAAEGGLVEERKAFLPQSYSLWKDSIEGRLWSLLTLFGLFISSP
FLFAFVALSVLSAVVRKLLRLPAARKLPEGSNKGRGRTALVTGGKMTKSLDVCRHLKNEGFRVIL
TETPRYWMSASRFSSAVDKFVVLPVAPETHPEGYVEALRNLFEKENVSLFAPVCSPFSSLYDAKA
AESLPEGAISWSLPAEMVQQLDDKVEFARMAKEVGLPVPDTLRVESKEEVRRFNSELAEKWRRDS
SSAIASGAEKKKTDCRRYILKTLDYDPMRRLDLFTLPCGPKELEKYLDETTISPDRPWLVQEFLE
GREYSSCALSWKGKLLAFTDNEAVISCYNFKYAGRDKIQEWVRVFCEKYQLSGVICVDFFERADG
TQLAIECNPRFSSNMTAFYNNPRLGAAMADPDLALRSGVTETPLPSSKESNWTLVDLYFHSYTQM
MKNPLAAFTAAAGLLLVSEETKEKQDAYWAPEDPLPSLALHCFHMPALLVRNVWDGRKWAKIDFC
IGKMTEENGD
R1G4T9
(SEQ ID NO: 17)
EVKPNGKVAIVSGGKMTKAYVIARQLKAQGCRVVLLETSKYWMVASRASNCVDRFAMVPLPEKDL
AGYLDAVRALAIEEKADLFIPVTSPAASEYEAQVAPVLPAGCVSWSLDLETVRDLDDKTAFCSSA
ERLGLPAPRSHRVASDEEAHAFNEKLLAEAATATAGAETRYILKSLAYDSMHRLDLFTLPCAPDN
PWIIQTFVVGDEYSTCALVKEGRLLAFTDNRACLSCFNYTPARSEALRSWVRDFCAARRLSGVVC
IDFIVDAQSGTPYAIECNPRFSSNVLNLFWNPPFGGALFRPHKGGGVEAFFWPPPPPPPLQIWAL
LSKRPFSLRSAGALLSTVATKKDAYFDVADPLPFIAHLFVHIPALLARNLSTGNKWAKIDPCIGK
LTEENGD
A0A433W0B3
(SEQ ID NO: 18)
MLLPQSITPTMQIFAVFQNLGTLLLLAIAFPFNCIVVLTALLWNLVSKPFRDRGILPVHPKNIML
TGGKMTKALQLARSFHMVGHRVVLVETHKYWLTGHRFSNAVDRFYTVPAPEKDPEAYSQALLAIA
KQENIDVYVPVCSPVASYYDSVAKSVLSGCCEVFHFDAEVTQMLDDKYEFAEKARSLGLSVPKSF
KITNPEQVINFDFSDAERPYILKSIPYDSVRRLNLTKLPCATPAETAAFVNSLPISPEKPWIMQE
FIPGQEYCTHSTVRNGELRMHCCCESSAFQVNYENVDKPEILAWVRHFVKELGITGQASFDFIQA
EDGNVYAIECNPRTHSAITMFYNHPGVADAFCRDVTCNTSTSRAGLLNSSFINNISGEPAPTIYP
LQPLSTSKPTYWTYHELWRLTGIRSFPQLQTWCKNILRGKDAIFAIDDPLPFLMVHHWQIPLLLL
DNLRRLKGWIRIDFNIGKIVELGGD
A0A139WZN8
(SEQ ID NO: 19)
MTQSISFSSPVPATPPISVKARFIALFQNLGTLTLLLLALPVNAVIVVISLVWNSLTRLFSTQQT
TVARSKNILISGGKMTKALQLARSFGAAGHRVVLIETHKYWLSGHRFSNAVSRFYTTPTPQYDPE
AYIQTLIDIVKRENIDVYVPVTSPVASYYDSLAKPALSPYCEVLHFDADVTKMLDDKFAFSEKAR
DLGLSVPKSFKITNPEQVLNFDFSQETRKYILKSIPYDSVRRLDLTKLPCDTLEETAAFVKSLPI
SPEKPWIMQEFIPGKEFCTHSTVRNGELRLHCCSESSAFQVNYENVENPEIQAWVKHFVNGLGFT
GQVSFDFIQTDDGKVYAIECNPRTHSAITMFYNHPQVSDAYLGTEPLTEPLQPLPNSKPTYWLYH
EVWRLTGIRSFSQLQNWVRNIFRGTDAIYKLHDPLPFLTVHHWQIPLLLLNNLWQLRGWTKIDFN
IGKLVEFGGD
A0A2Z5X784
(SEQ ID NO: 20)
MLCPYERLVFCLKEKLMTQSIPLSFSQPTTPLTVVKTKIVALFKTLGTLALLLLALPLNGFVVLI
SLLWVIVRNPFTKPTAVAAHPQNILVSGAKMTKALQLARSFHAAGNRVILIEGHKYWLSGHRFSN
AVSRFYTVPAPQDDPESYTQALLEIVKKEKIDVYIPVCSPVASYYDSLAKPVLSEYCEVFHFDAD
ITAMLDDKFAFTDQARSLGLSVPKSFKITDPEQIINFDFSQETRKYIIKSISYDSVRRLNLTKLP
CDTPEETAAFVRSLPISPEKPWIMQEFIPGKELCTHSTVRDGELRLHCCSNSSAFQINYENVENP
QIREWVQHFVKSLRLTGQVSFDFIQAEDGTVYAIECNPRTHSAITMFYNHPGVAQAYLGKTPQAA
PLEPLADSKPTYWLYHEIWRLTSIRSWKHLQTWFKNLVRGTDAIYSMDDPIPFLTLHHWQITLLL
LQNLQQLKGWVKIDFN
A0A1Z4GTP3
(SEQ ID NO: 21)
MAQSISLSLPSSTTPSTGVRVKIVALFKTLGTLTLLLIALPFNALIVLIALLWGIARSPFTKKAV
VAANPQTILVSGAKMTKALQLARSFHAAGHRVILIEGHKYWLSGHRFSQAVSRFYTVPAPQSDPE
AYIQALVEIVKKEKVDIYVPVCSPVASYYDSLAKPTLSEYCEVFHFDADITKMLDDKFAFTDKAR
SLGLSVPKSFKITDPQQVINFDFSQETRKYILKSIAYDSVRRLDLTKLPCDSPEETAAFVNSLPI
SPENPWIMQEFIPGKEFCTHSTVRDGELRLHCCCHSSAFQINYENVENPQIREWVQQFVKSLRLT
GQVSFDFIQAEDGTVYAIECNPRTHSAITMFYNHPGVAEAYFGKTPLAAPLEPLASSKPTYWIYH
EIWRLTNIRSWKQLQTRLNILFRGTDAIFRLNDPVPFLTLHHWQIPLLLLQNLQKLKGWVKIDFN
IGKLVELGGD
A0A1Q4RU46
(SEQ ID NO: 22)
MAQSISLSSPAKTHAPGISASSLKTLGTLTLLLLALPLNASLVLVALLLKSLRPQNVTTEEPKNI
LISGGKMTKALQLARSFHEQGHRVILLEAHKYWLTGHRFSFAVNKFYTVEAPEKDPEGYIQSLVN
IVEKENIDVYVPVCSPVASYYDSLAKKALPQCEVIHCDAEMTQMLDDKYAFAQTAQSFGLSVPKS
FKITEPEQVINFDFSQEKRKYILKSIPYDSVRRLDLTKLPCDTPEATAAFVRSLPISPEKPWIMQ
EFIPGKEYCTHSTVRNGVITLHCCCESSAFQVNYENVDNPKIFEWVSRFVKELGITGQVSFDFIE
AEDGNIYAIECNPRTHSAITMFYNHPGVADAYLGTGSNLAEPIQPKSTSKPTYWTYHEVWRLITT
RSWSDFVYRFKIITHGKDAIFSWQDPLPFLMNPHWQIFLLLIQNLQKNRGWVRIDFNIGKLVELG
GD
A0A0C2R3C6
(SEQ ID NO: 23)
MAQSLPLTSAGGATSPTAFVAQVKALFQNIATLTILLLVLPINAAIVLTSLFWSRVSRFVRPQTV
VAANRKNILISGGKMTKALQIARSFHAAGHRVVLIETHKYWLSGHRFSDAISRFYTTPTPQYDPE
AYIQALLDIVKKENIDVYVPVTSPVASYYDSLAKPALSPYCEVFHFDADVTQMLDDKFAFSEKAR
SFGLSVPKSFKITNPEQVLNFDFSGETRKYILKSIPYDSVRRLDLTKLPCDTPEETAAFVRSLPI
SPEKPWIMQEFIPGKEFCTHSTVKNGELRLHCCAESSAFQVNYENVENPKIQEWVRHFVKELGIT
GQVSFDFIQAEDGTVYAIECNPRTHSAITMFYNHPDVADAYLSEEPFTEPLVPLPNSKPTYWTYH
EVWRLTGIHSFAQLQTWIRNFLQGTDAIYQLDDPLPFLMVHHWQIPLLLLNNLRQLKGWTKIDFN
IGKLVEIGGD
A0A2R5FKA4
(SEQ ID NO: 24)
MRKYIFVVFQNLGTLVLLAIAFPLNCIVVLTSLLWNFLKQPFNKSIVVNPNSKNILIAGARMTKT
LQLARSFHAAGHRVIIIDIEKFWSSGNKYSNSVAGFYTVPDPSKDLEGYVESLHAIAKTEKIDFF
IPVAIFSVIHYDQGQPPLPDFVEFFHFDADVTKILDDKFAFAETARSFGLSVPKSFKITHPEQVI
NFDFSHEKRKYILKSIPYDQIRRLNLTKLPCATSAETAAFVNSLPISEENPWIMQEFIPGKEYCT
HTTARDGESRMYCCCESSAFQVNYENVDQQEIMQWATHFTKELGKTGQLSFDFIQAEDGTVYAIE
CNPRTHSAITMFYNHPGVADAYLGKEPLAESLQPLADSKPTYWLYHEVWRLNEIRNFEQLQTWVR
NIRRGKEAIFEVSDPLPFLMVHHWQIPLLILDNLRRLKGWIRIDENMGELIE
A0A0M0SH70
(SEQ ID NO: 25)
MTQSISVASPAPKTQSVPLGLRISALWKNVGTLALLLLVLPINAVIVLVSLLLGHQSQAIATEPK
NILISGAKMTKALQLARSFHAAGHRVVLVETHKYWLTGHRFSKAVSRFYTVPTPQSDPEAYTQAL
LDIVKTENIDVYVPVCSPIASYYDSLAKPVLSKFCEVFHCDADVTQMLDDKYAFAEKARSLGLSV
PKSFKITDPEQILNFDFSQEKRQYILKSIPYDSVRRLDLTKLPCETPEATADFVNSLPISPQKPW
IMQEFIPGKEYCTHSTVRNGELRMHCCCESSAFQVNYENVDHPQILEWVRHFVKALGITGQVSFD
FIQAEDGTIYAIECNPRTHSAITMFYNHPHVADAYLSEIPQLEPIQPLTNSKPTYWTYHEIWRLT
GIRSFSQLQTWLKTFFGGKDAIYCFSDPLPFLTVHHWQIPLLLLQNLQQLKGWIRIDFNIGKLVE
FGGD
A0A2T1F866
(SEQ ID NO: 26)
MLLPQSITPTMQIFAVFQNLGTLLLLAIAFPFNCIVVLTALLGNLVSKPFRDRGILPVSHPKNIM
LTGGKMTKALQLARSFHMVGHRVVLVETHKYWLTGHRFSNAVDRFYTVPAPEKDPEGYSQALLAI
AKQENIDVYVPVCSPVASYYDSVAKSVLSGCCEVFHFDAEVTQMLDDKYEFAEKARSLGLSVPKS
FKITNPEQVINFDFSDAERPYILKSIPYDSVRRLNLTKLPCATPAETAAFVNSLPISPEKPWIMQ
EFIPGQEYCTHSTVRNGELRMHCCCESSAFQVNYENVDKPEILAWVRHFVKELGITGQASFDFIQ
AEDGNVYAIECNPRTHSAITMFYNHPGVADAFCRDVTCNTSTSRAGLLNSSFINNISGEPAPTIY
PLQPLSTSKPTYWTYHELWRLTGIRSFPQLQTWCKNILRGKDAIFAIDDPLPFLMVHHWQIPLLL
LDNLRRLKGWIRIDFNIGKIVELGGD
A0A367QNV7
(SEQ ID NO: 27)
MAQSISVSSSPAIPSFPSETKIAVIIQNLLTLALLLLALPINATIVLVTLLWHTISRPFQQPATK
AANPKNILISGGKMTKALQLARSCNAAGHRVVLIETHKYWLSGHRFSQAVDKFYTVPAPQENPER
YTQALIDIIKQENIDVYIPVTSPLGSYYDSLAKPLLSKYCEVFHFDADITERLDDKFAFAETARS
LGLSVPKSFKITKAEQVLNFDFSQESRKYILKSIPYDSVRRLDLTKLPCATPEETAAFVRSLPIS
PEKPWIMQEFIPGKEFCTHSTVRDGELRLHCCCESSAFQVNYENVENSQIREWVRHFVKELKLTG
QVSFDFIQAEDGKVYAIECNPRTHSAITTFYDHPQVAQAYLDNEPMAQTLQPLPSSKPTYWTYHE
VWRLTGIRSLTQFKKWIANIWRGTDAIYKSDDPLPFLMVHHWQIPLLLIKNLRQLKGWTRIDFNI
GKLVELGGD
A0A2N6JWS5
(SEQ ID NO: 28)
MAQLQSIQASIFAVLQNLGTLALLMIAFPFNCIVVLLSLLLNFLSRPFHKPVILTKNPRNIMIAG
ARMTKTLQLARSFHAAGHRVILVDTEKFWLSGNQFSHAVAGFYTVPDPHKDLEGYTQALRAIAKK
ENIDFFIPVAIFAVIYYDSMSQHQLFDCCEVFHFNADVTKMLDDKFAFAEKARSLSLSVPKSFKI
TAPEQILNFDFSNEKRKYILKSIPYDAVRRLNMTLLPCDTPEQTAAFVKSLPISEEKPWIMQEFI
PGKEYCTHSTVRDGKQTIYCCCESSAFQVNYENVDKPEILQWVNHFVKELGLTGQISFDFIQAVD
GTVYVIECNPRTHSAITMFYNHPGVADAYLSKQPLAEPLQPLSDSKPTYWLYHEVWRLNEIRSLK
QLQTWIKNILRGKDAIFTVNDPLPFLMVHHWQIPLLLLDNLRRLKGWIRIDENPLLSL
B4VP63
(SEQ ID NO: 29)
MTNSLILAVLQNLGTLTLLAIAFPFNLTVVVVALVWDSLTRPFQNPKVANPNPKTIMLTGGKMTK
SLQLARSFYADGHRVILVESHKYWLVGHRFSRAVDRFYTVPAPNKDPDGYMEGLLAIAKQENVDV
YVPVCSPVASYYDSLAKPVLSGCCEVFHFDPDVTQLLDDKFAFAQKAREFGLSVPKSFKITDPQQ
VIDFDFRGEKRKYILKSIPYDSVRRLNLTKLPCKTPSETAAFVKSLPISEDKPWIMQEFIPGKEY
CTHSTVRNGELRLHCCCESSAFQVNYENVDQPDILQWVSRFVQGLNLTGQASFDFIKTEDGIVYA
IECNPRTHSAITMFYNHPGVAEAYLSDTPLPEPLQPLPESKPTYWLYHEVWRLNEIRSFGDIRRW
FKTVFGGKDAIFQVNDPLPFLMVHHWQIPLLLLDNLRRMQGWIRIDFNIGKLVELGGD
K9QUQ5
(SEQ ID NO: 30)
MAQSISFDSSPATPSLGLETKIAAIIQNILTLALLLLALPINAIIVCIALVLGTIFRPQTTKTSN
PKNILISGGKMTKALQLARSFHADGHRVVLLETHKYWLTGHRFSQAVDKFYTTPAPQKKPEDYIK
ALVDIVKRENIDVYIPVTSPVGSYYDSLAKPELSHHCEVFHFDAEITQMLDDKFAMAEKARSLGL
SVPKSFKITSGEQVINFDFSRETRKYILKSIAYDSVRRLDLTKLPCATPEETAAFVRKLPISPEK
PWIMQEFIPGKEFCTHSTVRDGEIRLHCCCESSAFQVNYENIENPQILEWVRHFVKELKLTGQIS
FDFIQTEDGQVYAIECNPRTHSAITTFYNHPQVAEAYIGKQPMAETLQPLATSKPTYWTYHEIWR
LTGIRSFTQLKTWLKNIWRGTDAILQLHDPLPFLMVHHWQIPLLLLNNLRQLKGWTRIDFNIGKL
VEFGGD
A0A0S3U2V2
(SEQ ID NO: 31)
MLNKLIAALQNLLTLTALLITLPINLAIVLIASLIGLFQRETIPQSNSPKRILITGGKMTKALQL
ARSFHAAGHFVVLVETQKYWLTGHQFSNAVDRFYTVPAPKQDSEAFIQALVDIVQRENIDFFVPV
TSPIESYYCSLAKPELSKYCEVLHFDVGITQLLDDKFELSEKARSLNLTAPKTYRITDPQQVLDF
EFDSSQYILKSIAYNSVHRLDMTKYPLESKAAMKAHLATLPISEDNPWILQEFISGQEYCTHSTV
RDGKVRLHCCAKSSAFQVNYEQVENSEIQAWVTTFVKALNLSGQISFDFIESSSGEVYAIECNPR
THSAITMFYNHPDVAKAYLGEPLTVEPIQPLPTSKPTYWTYHEVWRLITGDRPLYRLQTILHGKD
AILQTSDPIPFLMVHHWQIPLLLLNNLRHLKGWVRIDFNIGKLVELGGD
K9TVZ3
(SEQ ID NO: 32)
MLLPQSITPTMQIFAVFQNLGTLLLLAIAFPFNCIVVLTALLWNLVSKPFRDRGILPVSHPKNIM
LTGGKMTKALQLARSFHMVGHRVVLVETHKYWLTGHRFSNAVDRFYTVPAPEKDPEAYSQALLAI
AKQENIDVYVPVCSPVASYYDSVAKSVLSGCCEVFHFDAEVTQMLDDKYEFAEKARSLGLSVPKS
FKITNPEQVINFDFSDAERPYILKSIPYDSVRRLNLTKLPCATPAETAAFVNSLPISPEKPWIMQ
EFIPGQEYCTHSTVRNGELRMHCCCESSAFQVNYENVDKPEIIAWVRHFVKELGITGQASFDFIQ
AEDGNVYAIECNPRTHSAITMFYNHPGVADAFCRDVTCNTSTSRAGLLNSSFINNISGEPAPTIY
PLQPLSTSKPTYWTYHELWRLTGIRSFPQLQTWCKNILRGKDAIFAIDDPLPFLMVHHWQIPLLL
LDNLRRLKGWIRIDFNIGKIVELGGD
A0A2N6MZD6
(SEQ ID NO: 33)
MAQLQSIQASIFAVLQNLGTLALLMIAFPFNCIVVLLSLLLNFLSRPFHKPVILTKNPRNIMIAG
ARMTKTLQLARSFHAAGHRVILVDTEKFWLSGNQFSHAVAGFYTVPDPHKDLEGYTQALRAIAKK
ENIDFFIPVAIFAVIYYDLMSQHPLFDCCEVFHFNADVTKMLDDKFAFAEKARLLSLSVPKSFKI
TAPEQILDFDFSNEKRKYILKSIPYDAVRRLNMTLLPCDTPEQTAAFVKSLPISEEKPWIMQEFI
PGKEYCTHSTVRDGKQTIYCCCESSAFQVNYENVDKPEILQWVNHFVKELGLTGQISFDFIQAVD
GTVYAIECNPRTHSAITMFYNHPGVADAYLSKQPLAEPLQPLSDSKPTYWLYHEVWRLNEIRSLK
QLQTWVKNILRGKDAIFTVNDPLPFLMVHHWQIPLLLLDNLRRLKGWIRIDFNIGELIE
A0A218PXL8
(SEQ ID NO: 34)
MAQSISLSLAKSPGSSTGVWVKLVALFKTLGTLTLLLIALPFNALIVLISLLWGFVRSPFRQKAV
VADHPQTILVSGAKMTKALQLARCFHAAGHRVILIEGHKYWLSGHRFSKAVSGFYTVPAPELDPL
GYIQALVEIVKKEKVDVYVPVCSPVASYYDSLAKPALSEYCEVFHFDADVTKMLDDKFAFTDQAR
SLGLSVPKSFKITDHQQVINFDFSQETHKYILKNIAYDSVRRLNLTKLPCDTPEETAAFVNSLPI
SEENPWIMQEFIPGKELCTHSTVRDGELRLHCCSDSSAFQINYENVENPQIREWVQHFVKSLALT
GQVSFDFIQAESGTVYAIECNPRTHSAITMFYNHPGVAEAYLGKTPLTDLTEPLANSKPTYWIYH
EIWRLTGIRSWKQLQTSINTLAQGTDAVYQLDDPIPFLTLHHWQIPLLLLKNLQQLKGWVKIDFN
IGKLVELGGD
A0A1Z4HW63
(SEQ ID NO: 35)
MAQSISLSLPESTTPATSVGVKIAALFKTLGTLTLLLIALPFNALIVLIALLWGIVRSPFTKKAV
VAAHSQTILVSGAKMTKALQLARSFHAAGHRVILIEGHKYWLSGHRFSQAVSRFYTVPAPQSDSE
GYIQALVEIVKQEKVDIYVPVCSPIASYYDSLAKPALSEYCEVFHFDADITKMLDDKFAFTDKAR
SLGLSVPKSFKITDPQQVINFDFSQETRKYILKSIAYDSVRRLDLTKLPCNTSEETAAFVNSLPI
SPENPWIMQEFIPGKEFCTHSTVRDGELRLHCCCHSSAFQINYENVENPQICEWVQQFVKSLQLT
GQVSFDFIQAEDGSVYAIECNPRTHSAITMFYNHHGVADAYFGKTPLAAPLEPLASSKPTYWIYH
EIWRLTGIRSWKQLQTSVNTLLRGTDAIYNLNDPVPFLTLHHWQIPLLLLKNLQQLKGWVKIDFN
IGKLVELGGD
A0A1Z4LYV8
(SEQ ID NO: 36)
MAQSSVSVSASQPIAPPTSIGMRFFALFQNLATLTLLLLALPINATIVLTTLLLNILTSPFQKKQ
TTVVATEKKNILISGGKMTKALQLARFFHSAGHRVILTETHKYWLSGHRFSQSVDKFYTTPVPQK
DSQAYTQALIDIINKEGIDIYIPVTSPIASYYDSLAKPALSEYCEVFHIDAATCEMLDDKFAFSE
KARSFGLSIPKCFKITNPEQVINFDFSGETRKYILKSIPYDSVRRLDLTKLPCDTPEETEAFVRS
LPISPQKPWIMQEFIPGKEYCTHSTVRDGVMRLHCCCESSAFQVNYENVENPKIREWVTHFVKEL
GVTGQLSFDFIEAEDGNVYAIECNPRTHSAITIFHDQLQQAANAYLSKEPIAAPLQALPNSKPTY
WTYHEFWRLNEIRSLSQLGNWIKNMLRGTDAIYTFDDCLPFLMVHHWQIPVLLLKNLSKLKGWTR
IDFNIGKLVELGGD
A0A654SJH1
(SEQ ID NO: 37)
MAKSVSLSLAKSTTPSTDVRLKLVALFKTLGTLTLLLIALPENGLIVLIALLWGIVQWPLRKKAL
VAADPRTVLVSGGKMTKALQLARCFHGAGHRVILIETHKYWLSGHKFSRAVSAFYTVPSPQSDPE
GYIQSLVAIVKKEKVDFYVPVCSPVASYYDSLAKPALSAYCEVFHFDADITKMLDDKFAFTEQGR
SLGLSVPKSFQITDPQQVINFDFSQETRKYILKNIAYDSVRRLNLTKLPCNTPEETAAFVNSLPI
SAQNPWIMQEFIPGKELCTHSTVRDGELRLHCCSNSSAFQINYQNVENPQIRQWVQQFVKSLGLT
GQVSFDFIQAEDGTVYAIECNPRTHSAITMFYNHPGVADAYLGKTPQAAPVEPLANSKPTYWLYH
EIWRLTGIRSWKQLQTSVNTLVGGTDAIFCFDDPVPFLTLYHWQIPLLLLKNLQDLKGWVKIDFN
IGKLVELDGD
A0A2C6VZE1
(SEQ ID NO: 38)
MAQSISVSSSPAIPSFPSETKIAVIIQNLLTLALLLLALPFNATIVLVTLLWHTISRPFQQATTK
TANPKNVLISGAKMTKALQLARSFNAAGHRVVLIETHKYWLSGHRFSQAVDKFYTVPAPQENPER
YTQALIDIIKQENIDVYVPVTSPLGSYYDSLAKPMLSNYCEVFHFDADITQKLDDKFAFAETARS
LGLSVPKSFKITSAEQVLNFDFSQESRKYILKSIPYDSVRRLDLTKLPCATPEETAAFVKSLPIS
PEKPWIMQEFIPGKEFCTHSTVRNGELRLHCCCESSAFQVNYENVENSQIREWVRHFVKEQKLTG
QVSFDFIQAEDGRVYAIECNPRTHSAITTFYDHPQVAQAYLDKEPMAETLQPLPTSKPTYWTYHE
VWRLTGIRSFTQLKKWIANIWRGTDAIYKPDDPLPFLMVHHWQIPLLLLKNLRQLKGWTRIDFNI
GKLVELGGD
A0A2T1EQS1
(SEQ ID NO: 39)
MLALFQNLGTLLLLALAFPFNCIVVLVALLTKPKLPQATVAKAQNILISGGKMTKALQLARSFYA
AGHRVVLIETDKYWLTGHRFSRAVDAFYTVPAPQKDPEAYIQALVNIAKKENIDVYIPVCSPISS
YYDSLAKPALAGCCEVFHFDADITKMLDDKFAFAQTAQSFGLSVPKSYKITHPQQVLDFDFSTEQ
NKYILKSIPYDSVRRLNLTKLPCNTRAETAAFVNSLPISEEKPWIMQEFITGKEYCTHSTVRDGE
LRLHCCCESSAFQVNYENVDQPEILQWVSHFVKQLGVTGQASFDFIRAENGNIYAIECNPRTHSA
ITMFYNHPGVASAYLSSQPLKPLQPLTDSKPTYWLYHEVWRLNEIRSLQQLQTWFKNIRRGKESI
FAFNDPLPFLMVHHWQIPLLLLDNLRRLAGWIRIDFNIGKLVEFGGD
A0A1E5QWM1
(SEQ ID NO: 40)
MFSTTFKSLGTLALLKLALPFNLTLVLIASIINIFSTPFKIKKKPNINSKTVLLTGGKMTKALQL
ARSFYSAGHRVILVETHKYWLSGHRFSVAVDKFFTIPDPVKDKEGYIDGLLDIVKRENVDIFIPV
SSPVASYYDSVAKMVLSPYCKVLHFDVEMTLVLDDKASLCQKASSLGLTSPASYLITDVQEILDF
DFSKNNHKYILKSIKYDSVYRLNMTQFPFEGMEEYVRSLPISEENPWVMQQFITGQEYCTHSTVL
NGKIRLHCCSMSSHFQVNYEHVDNQKIYEWVEEFVGKLNLTGQISFDFIQTDDGTVYPIECNPRT
HSAISMFYNHPLVADAYLNDGDDAPITPLESSKPTFWTYHELWRLTEVRSPQDLSQWWQKVTKGQ
DGIFSWQDPLPFLMVHHWQIPLLLFGNLIKLKPWVKIDFNIGKLVESAGD
A0A218ACV8
(SEQ ID NO: 41)
MAQSISFDSSPATPSLGLETKIAAIIQNILTLTLLLLALPINTAIVFIYLVVGAIFRPQTSKTSN
PKNILISGGKMTKSLQLARSFHAPGHRVVLVETHKYWLTGHRFSQAVDKFYTTPAPQKDPEAYIQ
ALEEIVKRENIDVYIPVTSPVGSYYDSLAKPKLSPHCEVLHFDAEITQMLDDKFAMAEKARSLGL
SVPKSFKITSSEQVINFDFSGETRKYILKSIPYDSVRRLDLTKLPCATPEETAAFVRNLPISPEK
PWIMQEFIPGKEFCTHSTVRDGEIKLHCCCESSAFQVNYENVENPQILEWVKHFVKELKLTGQIS
FDFIQTEDGQVYAIECNPRTHSAITAFYNHPLVAEAYIGSVTETLQPLSTSKPTYWTYHEVWRLT
GIRSFTQLKTWLHNIWRGTDAILKLDDPLPFLMVHHWQIPLLLLNNLRQLKGWTRIDFNIGKLVE
LGGD
A0A2D3HK59
(SEQ ID NO: 42)
MRKHIFVVFQNLGTLVLLAIAFPLNCIVVLTSLLWSFIKQPFNKSIVVNPNSKNILIAGARMTKT
LQLARSFHAAGHRVIIIDIEKYWLSGNKYSNSVAGFYTVPDPSKDLEGYVETLHAIANTEKIDFF
IPVAIFSVIHYDQGKPPLPDCVEFFHFDADVTKILDDKFAFAETARSFGLSVPKSFKITDPEQVL
NFDFSQEKRKYILKSIPYDQVRRLNLTKLPCDTKSETAAFVKSLPISEENPWIMQEFIPGKEYCT
HTTARDGESRMYCCCESSAFQVNYENVDQREIMQWASHFTKELGKTGQLSFDFIQAEDGTVYAIE
CNPRTHSAITMFYNHPGVADAYLGKEPLAESLQPLPDSKPTYWLYHEVWRLNEIRSFKQLQTWVR
NIRRGKEAIFEVSDPLPFLMVHHWQIPLLILDNLRRLKGWIRIDFNMGELIE
A0A2S6VI18
(SEQ ID NO: 43)
MKSRQTPRERTFALLKSLGTLSLLLLAFPFSLSAVVGALLWSSLASLFQKRRVQAEPKRILLTGA
KMTKCLTLARSFHAAGHQVVMVETHKYWLSGNRFSNCVEAFYTVPAPQHDAEGYIQGLLNIVKQE
KIDMFIPVSSPVASYYDSLAKPALSPYCEVFAFDAETTKLLDNKFTFNQKAHSVGLSAPKTFLIT
NPEQVLNFDFAADGSQYILKSIAYDSINRLALLKLPCAPQKMAEYVRSLPISEENPWIMQEFLKG
QEYCTHAVVRDGKLLLYACSKSCDFLVNYEHDYNPAILDWVTRFVKELNLTGQICLDFIQAEDGT
VYPIECNPRTSTCITMFHDQPKVVADAYLSSGAQASKEPVQPLPDSKPTYWTFHELWRLLTKVKS
WKDLQYRLGIIFNGVDPVFHPRDPLPFLGVNHWQIPLLILNNVRQLKGWERIDFNIGKLVQLGGD
K9X913
(SEQ ID NO: 44)
MQSGQTTSERTFALLKSLGTLTLLLLAFPFSLSVVVGALLWSSLTSLFQKRRVQVEPKRILLTGA
KMTKCLTLARSFHAAGHQVFMVETKKYWLSGNQFSNCVEALYTVPAPQHDAEGYIQGLLNIVKQE
KIDMFIPVSSPVASYYDSLAKPALSPYCEVFAFDAETTKLLDNKFTFNQKAHSVGLSAPKTFLIT
NPEQVLNFDFAADGSQYILKSIAYDSINRLALLKLPCAPEKMAEYVHSLPISAENPWIMQEFLKG
QEYCTHAVVRDGKLLLYACSKSCDFLVNYEHDYNPAILDWVTRFVKELNLTGQICLDFIQAEDGT
VYPIECNPRTSTCITMFHDQPKVVADAYLSSSAQAPKEPVQPLPESKPTYWTFHELWRLLTKVKS
WKDLQYRLGIIFNGVDPVFHPRDPLPFLGVNHWQIPLLILNNVRQLKGWERIDFNIGKLVQLGGD
A0A1Y0RL91
(SEQ ID NO: 45)
MAHSISLSSRPATPAISIKALLVALFQNLGTLTILLLVLPINAAIVLISLLWSRLSSPWRSQKAV
VATHRKNILISGGKMTKALQLARSFHAAGHRVVLIETHKYWLSGHRFSNAVSRFYTTPTPQHNPE
AYIQALLDIVKREKIDVYVPVTSPVASYYDSLAKPALSPYCEVFHFDADVTQMLDDKFAFSEKAR
ALGLSVPKSFKITNPEQVINFDFSQETRKYILKSIPYDSVRRLDLTKLPCDTPEETAAFVRSLPI
SPEKPWIMQEFIPGKEFCTHSTVKNGELRLHCCSESSAFQVNYENIENPKIQKWVTHFVKELGIT
GQISFDFIQAEDGTVYAIECNPRTHSAITMFYNHPQVADAYLSQEAFTEPQEPLPNSKPTYWTYH
EVWRLTGIRSFAQLQTWIRNFLRGKDAIYQVDDPLPFLMVHHWQIFLLLLDNLRQFRGWTRIDFN
IGKLVELGGD
A0A2P8QMI8
(SEQ ID NO: 46)
MQIFAVFQNLGTLLLLAIAFPFNCIVVLTALFWNLVSKPFRDRGILPVSHPKNIMLTGGKMTKAL
QLARSFHMVGHRVVLVETHKYWLTGHRFSNAVDRFYTVPAPEKDPEGYSQALLAIAKQENIDVYV
PVCSPVASYYDSVAKSVLSGCCEVFHFDAEVTQMLDDKYEFAEKARSLGLSVPKSFKITNPEQVI
NFDFSDAERPYILKSIPYDSVRRLNLTKLPCATQAETAAFVNSLPISPEKPWIMQEFIPGQEYCT
HSTVRNGELRMHCCCESSAFQVNYENVDKPEILAWVRHFVKELGITGQASFDFIQAEDGNVYAIE
CNPRTHSAITMFYNHPGVADAFCRDVTCNVSTLYPLQPLSTSKPTYWTYHELWRLTGIRSFPQLQ
TWFKNILRGKDAIFAIDDPLPFLMVHHWQIPLLLLDNLRRLKGWIRIDFNIGKIVELGGD
A0A6B3P645
(SEQ ID NO: 47)
MALILFVQGRAYALFQNLGTLILLLIVLPFNFLKVIPSLLWNFISQPFQKKVVAENPKNILITGA
KMTKCLQLARSFHAAGHKVFLLEANKYWLSGNRFSNAVTGFYTLPFPQKDWEGYSQGLLEIIKKE
KIDVFIPVSSPAGSYYESLAKPLISEHCEVLHFDAEITQLLDNKFTFIEKAKSFGLSVPKSFLIT
NPEQVLNFDFATDGSKYILKSIPYDSVRRLDMTKLPMNSKAEMEEFVNSLPISEQRPWIMQEFVK
GKEYCTHSTVRKGKVRLYCCCESSEFQVNYHHVDRPQIYQWVEKFVRELNITGQISFDFIQTEDG
RVYPIECNPRTHSAITTFYDHPGVADAYLKDSKDENEASLIPLPNSKPTYWTYHELWRLTGIRSL
GQLKTWINRIFQGTDGIFQINDPLPFLMVHHWQIPLLLLGNLQKLKGWVRIDFNIGKLVELGGD
A0A6B3MZW3
(SEQ ID NO: 48)
MGLISGSQKPIYTVLQNLGTLTLLLSVLPFNLLKVLPALLWNFLSKPFQKKLVVENSKNIILTGA
KMTKCLQLARSFQAAGHKVFMLETDKYWLSGNRFSNSVTGFYTVPNPKKDWNGYCQKLLDIVKKE
NIDVFIPVSSAVLNYYESLVKPILSEYCEVLHFDVEITKLLDNKFTFIEKAKSFGLTVPKSFLIT
KPEQIINFDFATDGSQYILKSIPYDSVRRLNMTKLPMKSVQEMSNFVKSLPINQEKPWIMQEFVK
GKEYCTHSTVRKGQIRLHCCCESSEFQVNYEHVDHPQIYEWIEKFVKELNLTGQISFDFIQTEDN
RVYPIECNPRTHSAITTFYNHPEVADAYLNDSQNDNESPITPLSNSKPTYWTYHELWRLTAIRSW
EQLKAWSKKITAGTDSIFQFNDPLPFLMVHHWQIPLLLLENLKKLKGWVMIDFNIGKLVELEED
A0A2K8WS68
(SEQ ID NO: 49)
MFLTTFKSLGTLALLKLALPFNLTLVLIASIINIFSNPFKIKKKPNINSKTVLLTGGKMTKALQL
ARSFHSAGHRVILVETHKYWLSGHRFSVAVDKFFTMPNPVKDKEGYIDGLLDIVKRESVDIFIPV
SSPVASYYDSVAKMVLSPYCEVLHFDVEMTLVLDDKANLCKKASSLGLTSPASYLITNVQEILDF
DFSKNNHKYILKSIKYDSVYRLNMTQFPFEGMEEYVRSLPISEENPWVMQQFITGQEYCTHSTVR
NGKIRLHCCSESSHFQVNYKHIDNQKIYEWVEEFVGKLNLTGQISFDFIQTDDGTVYPIECNPRT
HSAISMFYNHPLVADAYLNDGDDAPITPLESSKPTFWTYHELWRLTEVRSPQDLSQWWQKVTKGQ
DGIFSWQDPLPFLMVHHWQIPLLLFGNLMKLKPWVKIDFNIGKLVESAGD
A0A4Q9JE38
(SEQ ID NO: 50)
MTQSISVASVGQTTQSVTLGLRISALFKNLATLALLLLVLPINAAIVLVSLLLGSQSQAIATEPK
NILISGGKMTKALQLARSFHAAGHRVVLVETHKYWLTGHRFSKAVSRFYTLPTPQSDPEAYTQAL
LDIVQKENIDVYVPVCSPVASYYDSLAKPVLSKYCEVFHCDADVTQMLDDKYAFVEKARSLGLSV
PKSFKITDPEQVSNFDFSQEKRKYILKSIPYDSVRRLDLTKLPCETPEATADFVNSLPISSQKPW
IMQEFIPGKEFCTHSTVRNGELRMHCCCESSAFQVNYENVDHPQILEWVRHFVKALGITGQVSFD
FIEAQDGTIYAIECNPRTHSAITMFYNHPDVANAYLSEIPQVEPIQPLINSKPTYWTYHEIWRLT
GIRSFSQLQTWLKNFFGGKDAIYSLSDPLPFLTVHHWQIPLLLLQNLQQLKGWIRIDFNIGKLVE
FGGD
Q3M6C5
(SEQ ID NO: 51)
MAQSLPLSSAPATPSLPSQTKIAAIIQNICTLALLLLALPINATIVFISLLVFRPQKVKAANPQT
ILISGGKMTKALQLARSFHAAGHRVVLVETHKYWLTGHRFSQAVDKFYTVPAPQDNPQAYIQALV
DIVKQENIDVYIPVTSPVGSYYDSLAKPELSHYCEVFHFDADITQMLDDKFALTQKARSLGLSVP
KSFKITSPEQVINFDFSGETRKYILKSIPYDSVRRLDLTKLPCATPEETAAFVRSLPITPEKPWI
MQEFIPGKEFCTHSTVRNGELRLHCCCESSAFQVNYENVNNPQITEWVQHFVKELKLTGQISFDF
IQAEDGTVYAIECNPRTHSAITTFYDHPQVAEAYLSQAPTTETIQPLTTSKPTYWTYHEVWRLTG
IRSFTQLQRWLGNIWRGTDAIYQPDDPLPFLMVHHWQIPLLLLNNLRRLKGWTRIDFNIGKLVEL
GGD
A0A252E4S5
(SEQ ID NO: 52)
MAQSISLSLPESTTPSTSAGVKIVALFKTLGTLTLLLIALPFNALIVLIALLWGIVRRPFTKKAA
VAAHPQTILVSGAKMTKALQLARSFHAAGHRVILIEGHKYWLSGHRFSKAVSRFYTVPAPQKDPE
GYIQALVEIVKKEKVDVYVPVCSPVASYYDSLAKPALSEYCEVFHFDADITKMLDDKFAFTDKAR
SLGLSVPKSFKITDPQQVINFDFSQETRKYILKSIAYDSVRRLDLTKLPCDTPEETAAFVNSLPI
SSENPWIMQEFIPGKEFCTHSTVRDGELRLHCCCNSSAFQINYENVENPQIREWVQQFVKSLRLT
GQVSFDFIQAEDGTVYAIECNPRTHSAITMFYNHPGVADAYLGKTPLAAPLEPLASSKPTYWIYH
EIWRLTGIRSWKQLQTSINTLLRGTDAICCLDDPVPFLTLHHWQIPLLLLKNLQQLKGWVKIDFN
IGKLVELGGD
A0A367RKS4
(SEQ ID NO: 53)
MAQSISLSLPQSTTPSTGVKVKIVALFKTLGTLTLLLIALPFNALIVLISLLWGIGRSPFTKKAV
VATHPQTILVSGAKMTKALQLARSFHAAGHRVILIEGHKYWLSGHRFSKAVSRFYTVPAPQEDPE
GYIQALVEIVKQEKVDVYVPVCSPVASYYDSLAKPALSEYCEVFHFDADITKMLDDKFAFTDRAR
SLGLSVPKSFKITDPQQVINFDFSQEIRKYILKSISYDSVRRLDLTKLPCDTPEQTAAFVNSLPI
SPEKPWIMQEFIPGKELCTHSTVRNGELRLHCCSNSSAFQINYENVENPRIREWVQHFVKSLGLT
GQVSFDFIQAEDGTTYAIECNPRTHSAITMFYNHSGVANAYFGKTLLDAPLEPLASSKPTYWIYH
EIWRLTGIRSWKQLQTSVNTIVRGTDAIYCLDDPVPFLTLYHWQIPLLLLKNLQQLKGWVKIDFN
IGKLVELGGD
A0A1E2WNZ8
(SEQ ID NO: 54)
MAQSISLSLPESTTPSTGIRIKIVALFKTLGTLTLLLIALPINALIVLLSLLWSILFTKKPAVAA
HPQTILVSGGKMTKALQLARSFHAAGHRVILVEGHKYWLSGHRFSNAVSRFYTVPAPQDDPEGYI
QALLEIVKKEKVDIYVPVCSPVASYYDSLAKPSLSAYCEVFHFDAEITKMLDDKFAFTDQARSLG
LSVPKSFKITDAEQVINFDFSKETRKYIIKSISYDSVRRLNLTKLPCDTPEETAAFVKSLPISPE
KPWIMQEFIPGKELCTHSTVRDGELRLHCCSDSSAFQINYENVENPQIRQWVQHFVKSLGLTGQV
SFDFIQAEDGTAYAIECNPRTHSAITMFYNHPGVAEAYFGKTLLAAPLEPLADSKPTYWIYHEIW
RLTGIRSAKQLQTWFQRLVRGTDAIYQINDPIPFLTLHHWQITLLLLQNLQKLKGWVKIDFNIGK
LVELGGD
A0A1B2CWG9
(SEQ ID NO: 55)
MAQSIPFDSASPTPQVSWGVRISALWKTVGTLLLLFLALPVNASIVLISLLWGIFSKPFEKRVVA
AAPKNILISGGKMTKALQLARSFHAAGHRVVLVESHKYWLTGHQFSNAVSVFYTVSPPEKDPEGY
TQQLLDIVKKERIDVYVPVCSPVASYYDSLVKPALSQHCEVFHCDAEITQMLDDKYAFSEKARSF
GLSVPKSFKITNPEQVINFDFSQEKRKYILKSIPYDSVRRLNLTKLPCDTPEETAAFVRSLPISP
EKPWIMQEFIPGKEFCTHSTVRNGELRLHCCCESSAFQVNYENVNNPQILEWVKHFIKEMGITGQ
VSFDFIQTEDGTVYAIECNPRTHSAITMFYNHPGVADAYLGKIPLPEPLQPLADSKPTYWLYHEI
WRLTGIRSLSQFWTWLKNLMRGKDAIYQLNDPLPFLTVPHWQITLLLLQNLRQLRGWVKIDFNIG
KLVELGGD
A0A1UTHY56
(SEQ ID NO: 56)
MQSGQTIRERTFASLKSLGTLTLLLLAFPFSLSVVVGALLWSSLTSLFQKHRVQVKPKRILLTGA
KMTKCLTLARSFHAAGHQVFMVETKKYWLSGNQFSNCVEALYTVPAPQHDAEGYIQGLLNIVKQE
KIDMFIPVSSPVASYYDSLAKPALSPYCEVFAFDAETTKLLDNKFTFNQKAHSVGLSAPKTFLIT
NPEQVLNFDFATDGSQYILKSIAYDSINRLALLKLPCAPATMAKYVHSLPISEENPWIMQEFLKG
QEYCTHAVVREGKLMLYACSKSCDFLVNYEHDYNPAILDWVTRFVKALNLTGQICLDFIQAEDGT
VYPIECNPRTSTCITMFHDQPKVVADAYLSSSASILKEPVQPLPDSKPTYWTFHELWRLITKVKS
WQDLQYRLGIIFNGVDPVFHPRDPLPFLGVNHWQIPLLILNNVRQLKGWERIDFNIGKLVQLGGD
A0A1L9QXK4
(SEQ ID NO: 57)
MLIILFIQNHAYALFQNLSTFLLLTLLLPFNLLKILPVVLWNILTPIRAKPPGYEKPKNILITGA
KMSKSLQLARSENGSGHRVFLLEIHKYWLSGNRFSNAIKGFYTVPNPQKDWDGYQQAVLEIVQKE
NINLFIPVSSPAGSYDESRLKPILSPYCEVFHFNLDITELLDNKFTFIEKAKSLGLSVPQSFLIT
DSKQILDFDFAQDGSRYILKSIPYDSVRRLDMTKLPMKSEQEMEEFVKKLPITEDKPWIMQEFVQ
GKEYCTHSTVRKGKIRLHCCCESSEFQVNYDHVEEPEIYQWVETFVRALNLTGQISFDFIKTEDG
QVYPIECNPRTHSAITTFHDHPGVADAYLKDAEDETESPIFPLPDSKPTYWTYHELWRVTEIRSF
GQFQAWIKRITEGTDGIFQLNDPLPFLMVHHWQIPLLLLQNLKKMKGWVRIDFNIGKLVELDGD
A0A2L2NR98
(SEQ ID NO: 58)
MGQSISLSLPQSPTSSTSVRVKIIALFKTLGTLTLLLIALPFNALIVLISLLWGIVRWTLPRRRR
SLFTKNVVAAHPQTILVSGAKMTKALQLARSFHAAGHRVILIEGHKYWLSGHRFSKAVSRFYTVL
APQSDLEGYIQALVEIVKKEKVDVYVPVSSPVSSYYESLAKAALSEYCEVFHFDPDITKMLDDKF
ALTDRARSLGLSVPKSFKITDPQQVINFDFSQETRKYILKSIDYDSVRRLNLTKLPCDTPEETAA
FVNSLPISPEKPWIMQEFIPGKELCTHSTVRDGELRLHCCSDSSAFQINYENVENPQIREWVQHF
VKSLALTGQVSFDFIQAQDGTVYAIECNPRTHSAITMFYNHPGVADAYLGKTPLAAPLEPLASSK
PTYFIYHEIWRLTGIRSWKQLQTSVNTLVRGTDAIYSLDDPIPFLTLHHWQIPLLLLKNLQQLKG
WVKIDFNIGKLVELGGD
A0A2H2XFD9
(SEQ ID NO: 59)
MPQSISLTSSPTINQVNNKSVDISSSLKTLGTLTLLLLALPVNATLVLVALLLNSLRPRNITTAA
NPKNILISGGKMTKALQLARSFHNAGHRVVLLEAHKYWLTGHRFSFAVNKFYTVEAPEKDPEGYV
QSLVDIVNKENIDVYVPVCSPVASYYDSLAKKALSSQCEVIHCDALTTQMLDDKYAFTETARGFG
LSVPKSFKITDPEQVINFDFSQEKRKYILKSIPYDSVRRLDLTKLPCDTPEATAAFVRSLPISPE
KPWIMQEFIPGKEYCTHSTVRNGEITLHCCCESSAFQVNYAQVDNPQIFEWVRHFLKQLGITGQV
SFDFIEAEDGTVYAIECNPRTHSAITMFYNHPGVADAYLGTLNNLEEPIQPLPTSKPTYWIYHEM
WRLINAGSWSKFVERLQIITRGTDAIFSWQDPLPFLMNPHWQIFLLLIQNLQKNRGWIRIDFNIG
KLVELGGD
A0A533NZW2
(SEQ ID NO: 60)
MFLQAKIWAFFQNIGTLTLLLLALPFNAIVVLPCLLWSWIAKLFQKKVVAANPKNILITGGKMTK
ALQLARCFHAAGHTVFLVETHKYWLSGHRFSRAVKGFFTVPAPEKHANGYCQGLLDIVKQEKIDV
FIPVSSPVASYYDSIAKSLLSPHCEALTFDAEITEMLDNKFTFCQKARELGLTAPKAFLITDPEQ
VLNFDFAADGSRYILKSIAYNSVYRLDLTKLPMSSKEQMASFVKGLPISESQPWIMQEFISGQEY
CTHSTVRNGIVRLHCCSQSSPFQVNYEQVDNQNIFQWVQQFVKALNLTGQISLDVIQTKDGKVYP
VECNPRTHTAIAMFYNHPGVADAYILDSKDAREPPIQPLPESKPTYWTYHELWRLTGIRSWGQLK
GWFNKIIKGTDGIFQVNDPLPFLMVHHWQIPLLLLNNMRKFKGWVKIDFNIGKLVELGGD
A0A367RVN3
(SEQ ID NO: 61)
MAQSISLSLPQSPTSSTGIKVKLVALFNTLGTLTLLLIALPFNALIVLISLLWGIVSSPFTKKAV
VAAHPQTILVSGAKMTKALQLARSFHAAGHRVILIEGNKYWLSGHRFSKAVSRFYTVPAPQEDPE
GYIQALVEIVKREKVDVYVPVCSPVASYYDSLAKPLLSEYCEVFHFDPDITKMLDDKFAFTDRAR
SLGLSVPKSFKITDPQQVINFDFSQETRKYILKSIDYDSVRRLNLTKLPCDTPEETAAFVNSLPI
SAEKPWIMQEFIPGKELCTHSTVRNGELRLHCCSNSSAFQINYENVENPQIREWVQHFVKSLALT
GQVSFDFIQAEDGTAYAIECNPRTHSAITMFYNHPGVADAYLGKTPLAAPLEPLASSKPTYFLYH
EIWRLTGIRSWKQLQTSVNTLVRGTDAIYSLDDPIPFLTLHHWQIPLLLLKNLQQLKGWVKIDFN
IGKLVELGGD
A0A1Z4TPY4
(SEQ ID NO: 62)
MLMGFFEGEFMTQSISVASPAPKTQSVPLGFRISALWKNVGTLALLLLVLPINAVIVLVSLLLGH
QSQAIATEPKNILISGGKMTKALQLARSFHAAGHRVVLVETHKYWLTGHRFSKAVSRFYTLPTPQ
SDPKAYTQALLDIVKKENIDVYVPVCSPVASYYDSLAKPVLSKYCEVFHCDADVTQMLDDKYAFA
EKARSLGLSVPKSFKITDPEQVINFDFSQEKRQYILKSIPYDSVRRLDLTKLPCETPEVTADFVN
SLPISPQKPWIMQEFIPGKEFCTHSTVRNGELRMHCCCESSAFQVNYENVDHPQILEWVRHFVKE
LGITGQVSFDFIQAEDGTIYAIECNPRTHSAITMFYNHPSVADAYLSEIPQLEPIQPLFNSKPTY
WIYHEIWRLTGIRHWSQLQTWLKNFFGGKDAIYSFSDPLPFLTVHHWQIPLLLLQNLQQLKGWLR
IDFNIGKLVEFGGD
A0A6B3MAD2
(SEQ ID NO: 63)
MGLISRSQKPVYIALQNLGILTLLLSVLPFNLLKVLPAVLWNFISKPFQKKVVAENSKNIILTGA
KMTKCLQLARSFQVAGHKVFMLETDKYWLSGNRFSNTVTGFYTVPNPKKNWNGYCQELLDIVKRE
DIDVFIPVSGAALNYYESLIKPILSEHCEVLHFDIEITKLLDNKFTFIEKAKSFGLAVPKSFLIT
NPEQILNFDFPADGGQYILKSIPYDSVRRLDMRKLPMKSAQEMKDFVNSLPISEEKPWIMQEFVK
GKEYCTHSTVRKGQIRLHCCCESSEFQVNYEHVNHPQIYEWVETFVKELNLTGQISFDFIQTEDN
RVYPIECNPRTHSAITTFYNHPEVADAYLNDSQDDNESPLIPLPNSKPTYWIYHELWRLTAIRSW
EQLKDWIKKITAGTDSIFQFNDPLPFLMVHHWQIPLLLLDNLKKLKGWVMIDFNIGKLVELEED
A0A1Z4IH51
(SEQ ID NO: 64)
MTQSISLSLPESTTPSVGIKVKILALFKTLGTLSLLLVALPFNVLIVLISLLWGIVRVPFTKNVV
ATHSQTILVSGAKMTKALQLARSFHADGHRVILIESHKYWLSGHRFSKAVSRFYTVPSPQKDPES
YIQALIEIVKKEKVDVYVPVCSPVASYYDSLAKPALSEYCEVFHFNADITKMLDDKFAFTQKARA
LGLSVPKSFKITDPQQVINFDFSQETRKYILKSINYDSVRRLNLTKLLCDTPEETAAFVKSLPIS
PETPWIMQEFIPGKEFCTHSTVRDGELRLHCCCHSSAFQINYENVENPQIREWVQHFVKSLGLTG
QVSFDFIQAEDGTVYAIECNPRTHSAITMFYNHPGVAEAYFGKIPLPAPVEPLATSKPTYWTYHE
IWRLTGIRSWKQLQTAIKTIFQGTDAIYCLDDPLPFLTLHHWQIPLLLLQNLQQLKGWVKIDFNI
GKLVELGGD
A0A1Z4IB36
(SEQ ID NO: 65)
MAQSLSLSSSHATPSIPWQTRVAAILQNIGTLTLLLLALPINASIVFISWLIFRPQKVKAANPQN
ILISGGKMTKALQLARSFHAAGHRVVLLETHKYWLTGHRFSVAVDKFYTVPAPQENPQAYIQALV
DIVKQENIDVYVPVTSPAGSYYDSLAKPELSRYCEVFHFDADITQMLDDKFALVEKARSLGLSVP
KSFKITSPEQVINFDFSGESRKYILKSIPYDSVRRLDLTKLPCATPEETAAFVRTLPISQEKPWI
MQEFIPGKEFCTHSTVRDGELRLHCCCESSAFQVNYENVDNPQIREWVRRFVKELKLTGQISFDF
IQAEDGTVYAIECNPRTHSAITTFYDHPQVAQAYLSKETTAETLQPLATSKPTYWTYHEVWRLTG
IRSLTQLGRWLGNIWRGTDAIYQPGDPLPFLMVHHWQIPLLLLNNLRRLKGWTRIDFNIGKLVEL
GGD
K9VKW1
(SEQ ID NO: 66)
MLETVSVAAMPSERETNTGNRRFPTAFKTIATLILLLLVMPLNLALTAIALLRSIIIKPFQSRST
TATPQTILISGGKMTKALQLARSFHQAGHRVILVETEKYWLTGHRYSRAVDRFYTVPNPQTEEYP
QALLKIVRQEGVNVYVPVCSPVASYYDAEVKRVLSGHCTVMHVDVETLQRLDDKYEFATAAQALG
LPVPKSYRITNPQQVIDFDFSDAQRKYIIKSIPYDSVRRLDLTKLPCETPAETAAFVNSLPISES
KPWIMQEYIPGQEFCTHSTVRNGHLQLHCCCKSSAFQVNYENVDRPDIENWIRQFAKSLNLTGQV
SFDFIQAADDGEIYAIECNPRTHSAITMFYNHPDVAKAYLEPDPLPQTVQPLASSRPTYWIYHEI
WRLVTHLSSPKLVSERLKIIAQGKDAIFDWDDPLPFLMVHHWQIPLLLWGNLQNPKEWIRIDFNI
GKLVEIGGD
A0A2T1F5R3
(SEQ ID NO: 67)
SRSVDRFYTVPKPQEKDYIDALLEIVQREGVDVYIPVCSPVASYYDALAKQVLSKYCEVMHFDPE
LVQKLDDKSEFSAIATSLGLAVPDSYRITDTQQILDFDFAKQAHTYILKSIPYDSLRRLNLTQLP
CETPQQTAAFVEQLPICESNPWIMQAFITGQEYCTHSTVRNGELQLHCCCESSAFQINYEMVDKP
EIEAWVRKFVSSLKLTGQVSFDFIQTRDGGVYAIECNPRTHSAITMFYNHPDVARAYLESDFPLI
KPLESSRPTYWIYHEIWRLVTQPTQIGQRLKIIASGKDAIFDWADPLPFLMVHHAQIPWLLLENL
RQLKGWMRIDFNIGKLVEPAGD
K9W0D3
(SEQ ID NO: 68)
MAQVQPIKARIFAVFQNLGTLALLAIAFPINCIVVLASLLWNFCSRPFSKQGVSTLNPKNILIGG
GKMTKTLQLARLFHAAGHRVILFDSEKFRFSGYRFSNAVDRFYTVPDPQTDLEGYTQALRAIAKQ
ENIDIFIPVGIFAGGYFDSQRQPVLSGCCELFHFDADTMKMLDNKFTFGEIARSFGLSVPKTFLI
TDPEQVLQFDFANEKNKYILKSIVYDSVYRLDMTKLPMESQEKMAAHVNSLPIRKDNPWILQEFI
SGKEYCTHSTVRNGELTVHCCCESSAFQVNYENVDHPEIMQWVSRFVKELKLSGQISFDFMQAED
GTLYAIECNPRTHSAITMYYNHPDLADAYLSAERRNYALPLQPLPDSKPTYWLYHEVWRLNEIRS
LKQLQTWFKNIWRGKDAIFEVNDPLPFLMVHHCYIPLLLLDSLRKLKGWVRIDFNIGKLVQLEGD
A0A1Z4SWP6
(SEQ ID NO: 69)
MPQSISLTSSPTINQVNNKSVDISSSLKTLGTLTLLLLALPVNATLVLVALLLNSLRPRNITTAA
NPKNILISGGKMTKALQLARSFHNAGHRVVLLEAHKYWLTGHRFSFAVNKFYTVEAPEKDPEGYV
QSLVDIVNKENIDVYVPVCSPVASYYDSLAKKALSSQCEVIHCDALTTQMLDDKYAFTETARGFG
LSVPKSFKITDPEQVINFDFSQEKRKYILKSIPYDSVRRLDLTKLPCDTPEATAAFVRSLPISPE
KPWIMQEFIPGKEYCTHSTVRNGEITLHCCCESSAFQVNYAQVDNPQIFEWVRHFLKQLGITGQV
SFDFIEAEDGTVYAIECNPRTHSAITMFYNHPGVADAYLGTLNNLEEPIQPLPTSKPTYWIYHEM
WRLINAGSWSKFVERLQIITRGTDAIFSWQDPLPFLMNPHWQIFLLLIQNLQKNRGWIRIDFNIG
KLVELGGD
A0A1U71932
(SEQ ID NO: 70)
MAQSISVSSSPAMPSLAVETKIAVIIQNILTLALLLLALPINATIVVVTLLWCNISRPFQHSATK
AANPKNILISGGKMTKALQLARSFNAAGHRVVLIETHKYWLSGHRFSQAVDKFYTVPAPQENPEC
YTQALIDIIKQENIDVYIPVTSPLGSYYDSLAKPLLSEYCEVFHFDADITQKLDDKFAFAETARS
LGLSAPKSFKITSAEQVLNFDFSQESRKYILKSIPYDSVRRLDLTKLPCATPEETAAFVRSLPIS
PEKPWIMQEFIPGKEFCTHSTVRDGELRLHCCCESSAFQVNYENVENSQIREWVRHFVKELKLTG
QISFDFIQAEDGRVYAIECNPRTHSAITTFYDHPKVAQAYLDKEPMAETLQPLPTSQPTYWTYHE
VWRLTGIRSFTQLKKWIANIWRGTDAIYKSDDPLPFLMVHHWQIPLLLIDNLRRLKGWTRIDFNI
GKLVELGGD
A0A1W5CLX0
(SEQ ID NO: 71)
MAQSLPLSSAPATPSLPSQTKIAAIIQNICTLALLLLALPINATIVFISLLVFRPQKVKAANPQT
ILISGGKMTKALQLARSFHAAGHRVVLVETHKYWLTGHRFSQAVDKFYTVPAPQDNPQAYIQALV
DIVKQENIDVYIPVTSPVGSYYDSLAKPELSHYCEVFHFDADITQMLDDKFALTQKARSLGLSVP
KSFKITSPEQVINFDFSGETRKYILKSIPYDSVRRLDLTKLPCATPEETAAFVRSLPITPEKPWI
MQEFIPGKEFCTHSTVRNGELRLHCCCESSAFQVNYENVNNPQITEWVQHFVKELKLTGQISFDF
IQAEDGTVYAIECNPRTHSAITTFYDHPQVAEAYLSQAPTTETIQPLTTSKPTYWTYHEVWRLTG
IRSFTQLQRWLGNIWRGTDAIYQPDDPLPFLMVHHWQIPLLLLNNLRRLKGWTRIDFNIGKLVEL
GGD
A0A328IAQ4
(SEQ ID NO: 72)
MTQSISVASVGQTTQSVTLGLRISALFKNLATLALLLLVLPINAVIVLVSVLLGSQSQAIATEPK
NILISGGKMTKALQLARSFHAAGHRVVLVETHKYWLTGHRFSKAVSRFYTLPTPQSDPQAYTQAL
LDIVKKESIDVYVPVCSPVASYYDSLAKPVLSKYCEVFHCDADVTQMLDDKYAFAEKARSLGLSV
PKSFKITDPEQVINFDFSQEKRQYILKSIPYDSVRRLDLTKLPCETPQATADFVNSLPISPQKPW
IMQEFIPGKEYCTHSTVRNGELRMHCCCESSAFQVNYENVDHPQILEWVRHFVKALGITGQVSFD
FIEAEDGTIYAIECNPRTHSAITMFYNHPDVANAYLSEIPQVEPIQPLTNSKPTYWTYHEIWRLT
GIRSFSQLQTWVKNFFGGKDAIYSLSDPLPFLAVHHWQIPLLLLQNLQQLKGWIRIDFNIGKLVE
FGGD
A0A533NF66
(SEQ ID NO: 73)
MFLQAKIWAFFQNIGTLTLLLLALPFNAIVVLPCLLWSWIAKLFQKKVVAANPKNILITGGKMTK
ALQLARCFHAAGHTVFLVETHKYWLSGHRFSRAVKGFFTVPAPEKHANGYCQGLLDIVKQEKIDV
FIPVSSPVASYYDSIAKSLLSPHCEALTFDAEITEMLDNKFTFCQKARELGLTAPKAFLITDPEQ
VLNFDFAADGSRYILKSIAYNSVYRLDLTKLPMSSKEQMASFVKGLPISESQPWIVQEFISGQEY
CTHSTVRNGIVRLHCCSQSSPFQVNYEQVDNQKIFQWVQQFVKALNLTGQISLDVIQTKDGKVYP
VECNPRTHTAIAMFYNHPGVADAYLLDSKDAREPPIQPLPESKPTYWTYHELWRLTGIRSWGQLK
GWFNKIIKGTDGIFQVNDPLPFLMVHHWQIPLLLLNNMRKFKGWVKIDFNIGKLVELGGD
A0A479ZZ55
(SEQ ID NO: 74)
MFPINLTLVITAFLTNLITLPFPKKITYENSKNILLTGGKMTKSLQLARSFHRAGHKVFMVETHK
YWLSGHQYSKAVKKFLTVPAPEKDPEGYCQSLLDIVKREKIDVFIPVSSPVASYYDSLAKPILSP
YCEVFHFDTEMTKTLDDKFSLCEQARVLGLTAPKVFLITSPGEIINFDFSQEQNPYIIKSIQYDS
VTRLDMTKFPFEGMKEYVKKLPISKERPWVMQEFIKGQEYCTHSTVRDGEIRLHCCSKSSPFQVN
YEQVDNPEIFQWVQKFVKELNLTGQISFDFMQTEDGKVYPIECNPRTHTAITMFYDHPGLADAYL
EPGKNQPHIEPLPTSKPTYWLYHELWRITGIRSFNDLTNWLNKVIKGKDAMLDKDDPLPFLMVHH
WQIVLLLLQNMVKLKGWVRIDFNIGKLVEIGGD
A0A357A498
(SEQ ID NO: 75)
MLIILFIQNRAYALFQNLSTFLLLTLLLPFNLLKILPALLWNILTSIRAKLPGDEKPKNILITGA
KMSKSLQLARSENGAGHRVFLLETHKYWLSGNRFSNAIKDFYTVPNSEKNWDGYQQAVLEIVQKE
NINLFIPVSSAAGSYDESRLKAILSPYCEVFHFDLDITELLDNKFTFIEKAKNLGLSVPKSFLMT
DSKQILDFDFVQDGSRYILKSIPYDSVRRLDMTKLPMKSEQEMEEFVKELPITEDKPWIMQEFVQ
GKEYCTHSTVRKGKIRLYCCCESSEFQVNYNHVEEPEIYQWVKTFVRALNLTGQISFDFIKTEDG
QVYPIECNPRTHSAITTFHDHPGVADAYLKDVEDETKSPIFPLPDSKPTYWTYHELWRLTQIRSF
GQFKAWIKRMIEGTDGIFQPHDPLPFLMVHHWQIPLLILQNLKTMKGWVRIDFNIGKLVELDGD
A0A1Z4QDW0
(SEQ ID NO: 76)
MAQSISVDSSPAIPSLASETKIAVIIQNILTLALLLLALPINATIVLVTLFWGTILRPFQHSATK
TANPKNILISGGKMTKALQLARSFHAAGHKVVLLETHKYWLTGHRFSQAVDKFYTVPAPQENPES
YTQALIDIIKQENIDVYIPVTSPLGSYYDSLAKPLLSRHCEVFHFDVDITQNLDDKFEFAQKARS
LNLSAPKSFKITSAEQVLNFDFSQESRKYILKSIPYDSVRRLDLTKLPCATPEETAAFVRSLPIS
PEKPWIMQEFIPGKEFCTHSTVRDGELRLHCCCESSAFQVNYENVENSQIREWVRHFVKELKLTG
QISFDFIQAEDGAVYAIECNPRTHSAITTFYDHPKVAQAYLDQEPMAETLQPLPTSKPTYWTYHE
VWRLTGIRSFTQLQKWLANIGRGTDAIYKLDDPLPFLMVHHWQIPLLLLNNLLRLKGWTRIDFNI
GKLVELGGD
K9R4C7
(SEQ ID NO: 77)
MAQSSIPVLSSQTATHTISLGRRFVALVQNLATLTALLLALPINATIVFISLVLKILISPFQKEQ
TTVTTAERKNILISGGKMTKALQLARFFHAAGHRVVLTETHKYWLSGHRFSQAVDKFYTTPVPQK
DSQIYTQALIDIVNKENIDIYIPVTSPIASYYDALAKQTLSEYCEVFHIDAATCEMLDDKFAFSE
KARSFGLSVPKSFKITNPEQVLNFDFSGETRKYILKSIPYDSVRRLDLTKLPCDTPEETEAFVRS
LPISPQKPWIMQEFIPGKEYCTHSTIRDGVVRLHCCCESSAFQVNYENVENAKIREWVTHFVKEL
GVTGQLSFDFIEAEDGNVYAIECNPRTHSAITIFHDQLQPAANAYLSKEPIKEPLQALINSKPTY
WTYHEFWRLNEIRSFSQLGNWIKNMLQGTDAIYTFDDSLPFLMVHHWQIPLLLLKNLFKLKGWTR
IDFNIGKLVESGGD
A0A3SOZZ73
(SEQ ID NO: 78)
MAQSISLTESQTTVKPLAVWGKINALLKNLGTLVLLLVALPINATIVLVSLLWNLLAKPFQKEQT
VAGDRKNILISGAKMTKALQLARSFHAAGHRVVLLETHKYWLSGHRFSKAVDNFYTTPVPQRDPQ
AYTQALIDIIEKENIDVYIPVTSPIASYYDSLAKPVLSQYCEVFHFDAAVTQMLDDKFAFSEKAR
SLGLSVPKSFKITSPEQVLNFDFSQETRKYILKSIPYDSVRRLDLTKLPCDTPEQTEAFVRSLPI
SAQKPWIMQEFIPGKEFCTHSTVRDGEIRLHCCCESSAFQVNYEHVEHPQISEWIARFVKGLGIT
GQISFDFIQAEDGSVYAIECNPRTHSAITTFHDRPEVAQAYLGKEAMTEPLQPLPSSKPTYWLYH
EVWRLTSIRSLAQLRTWIRNIWRGTDAIYKLDDPLPFLMLHHWQIPLLLLNNLWRLKGWTRIDFN
IGKLVELGGD
A0A3CONJT8
(SEQ ID NO: 79)
MAQLLFVRTPSFTMLKSLGTLTLLLIAFPINSIVVLTSLLWGLLSRPFQKQPLPADNQKTAMFTG
GKMTKALQLARSFHAAGHRVILVETHKYWLTGHRFSNAVDRFYTIPAPQKDPEGYTQALLNIAKQ
ENVDIYIPVCSPVSSYYDSLAKPALSGCCEVFHFDADITKMLDDKFAFSEKARALGLSVPKSFKI
TNPEQVLNFDFSNETRKYILKSIPYDSVRRLNLTKLPCDTPEETAAFVKSLPISEEKPWIMQEFI
PGQEYCTHSTVRDGELRLHCCCESSAFQVNYENVDQPEIMKWVSHFVKELKLTGQASFDFIQAED
GAIYAIECNPRTHSAITMFYNHPGVADAYLGKEPLAEPLQPLPDSKPTYWLYHEIWRLNEIRSWS
QLQTWMNNLLRGTDAIFDVNDPLPFLTVHHWQIPVLLLDNLRKLRGWVRIDFNIGKLVESGGD
B2J6X7
(SEQ ID NO: 80)
MAQSISLSLPQSTTPSKGVRLKIAALLKTIGTLILLLIALPLNALIVLISLMCRPFTKKPAVATH
PQNILVSGGKMTKALQLARSFHAAGHRVILIEGHKYWLSGHRFSNSVSRFYTVPAPQDDPEGYTQ
ALLEIVKREKIDVYVPVCSPVASYYDSLAKSALSEYCEVFHFDADITKMLDDKFAFTDRARSLGL
SAPKSFKITDPEQVINFDFSKETRKYILKSISYDSVRRLNLTKLPCDTPEETAAFVKSLPISPEK
PWIMQEFIPGKELCTHSTVRDGELRLHCCSNSSAFQINYENVENPQIQEWVQHFVKSLRLTGQIS
LDFIQAEDGTAYAIECNPRTHSAITMFYNHPGVAEAYLGKTPLAAPLEPLADSKPTYWIYHEIWR
LTGIRSGQQLQTWFGRLVRGTDAIYRLDDPIPFLTLHHWQITLLLLQNLQRLKGWVKIDFNIGKL
VELGGD
A0A0C1NCV3
(SEQ ID NO: 81)
MTKLQPIKARIIAVFQNLGTLLLLAIAFPINCSVVLVSLLWNFFSRPSHKQVVLTENPKNILIGG
GRMTKTLQLARSFHAAGHRVILVDIDKYWLSGHRFSRAVAGYYTVPAPQKDLEGYTQALRAIAKK
ENIDFFIPVAIFAVSYFDSKGEPVLSGCCEIFHFDADITKMLDDKFAFAEKARSLGLSVPKSFKI
TDPEQVLNFDFSQEKRKYILKSIPYDCLRRLNMTKLPCDTFDMTAEFVKSLPISEEKPWIMQEFI
PGKEYCTHSTVRDGELRLYCCCESSAFQVNYENVDRPEIRQWVQQFVQEVGLTGEISFDIIQADD
GTVYPIECNPRTHSAITMFYNHPGVANAYLNKEPLVEPLQPLADSKPTYWLYHEVWRLTGIRSLK
QLQTWIRNILRGKEAIFSVSDPLPFMMVHHWQIPLLLLDNLRRLKGWVRIDFNLGELIESEEY
A0A1Z4S904
(SEQ ID NO: 82)
MAQSISFSSAPATPSVPSTSKIAAIFPNIGTLTLLLLALPINASIVLITLLLRAILRPFQPSAVK
AANPKNILISGGKMTKALQLARSFHAAGHRVVLLETHKYWLTGHQYSQAVDKFYTVSAPQENPER
YTQALVDIIKQENIDVYIPVTSPLGSYYDSLAKPELSRYCEVFHFDADITQMLDDKYELAQTARS
LGLSVPKSFKITSAEQVLNFDFSGETRKYILKSIPYDSVRRLDLTKLPCATPEETAAFVRSLPIS
PEKPWIMQEFIPGKEFCTHSTVRNGELRLHCCCESSAFQVNYENVENPQILEWVKHFVKELKLTG
QISFDFIQAEDGKVYAIECNPRTHSAITTFYDHPKVAEAYLSQEATTETLQPLPTSKPTYWTYHE
VWRLTGIRSFKQLKTWIVNIWRGTDAIYKFDDPLPFLMVHHWQIPLLLLKNLRQLKGWTRIDFNI
GKLVELGGD
A0A2K8SZ63
(SEQ ID NO: 83)
MFQNLGTLVLLAIAFPLNCIVVLTSLLWSFIKQPFNKSIVVNPNSKNILIAGARMTKTLQLARSF
HAAGHRVIIIDIEKYWLSGNKYSNSVAGFYTVPDPSKDLEGYVETLHAIANTEKIDFFIPVAIFS
VIHYDQGKPPLPDCVEFFHFDADVTKILDDKFAFAETARSFGLSVPKSFKITDPEQVLNFDFSQE
KRKYILKSIPYDQVRRLNLTKLPCDTKSETAAFVKSLPISEENPWIMQEFIPGKEYCTHTTARDG
ESRMYCCCESSAFQVNYENVDQREIMQWASHFTKELGKTGQLSFDFIQAEDGTVYAIECNPRTHS
AITMFYNHPGVADAYLGKEPLAESLQPLPDSKPTYWLYHEVWRLNEIRSFKQLQTWVRNIRRGKE
AIFEVSDPLPFLMVHHWQIPLLILDNLRRLKGWIRIDENMGELIE
A0A3N6PGG7
(SEQ ID NO: 84)
MALILFVQGRAYALFQNLGTLILLLIVLPFNFLKVIPSLLWNFISQPFQKKVVAENPKNILITGA
KMTKCLQLARSFHAAGHKVFLLEANKYWLSGNRFSNAVTGFYTLPFPQKDWEGYSQGLLEIIKKE
KIDVFIPVSSPAGSYYESLAKPLISEHCEVLHFDAEITQLLDNKFTFIEKAKSFGLSVPKSFLIT
NPEQVLNFDFATDGSKYILKSIPYDSVRRLDMTKLPMNSKAEMEEFVNSLPISEQRPWIMQEFVK
GKEYCTHSTVRKGKVRLYCCCESSEFQVNYHHVDRPQIYQWVEKFVRELNITGQISFDFIQTEDG
RVYPIECNPRTHSAITTFYDHPGVADAYLKDSKDENEASLIPLPNSKPTYWTYHELWRLTGIRSL
GQLKTWINRIFQGTDGIFQINDPLPFLMVHHWQIPLLLLGNLQKLKGWVRIDFNIGKLVELGGD
A0A0C2QMV0
(SEQ ID NO: 85)
MKEQIFIVFQNLGTLVLLAIAFPFNCIVVLTSLVWNFIKQPFSQSIVVNPNSKNILIAGARMTKT
LQLARSFHAAGHRVIIIDIEKFWSSGNKYSNSVAGFYTVPDPSKDLEGYVESLHAIAKKEKIDFF
IPVAIFSVIHYDSQGKPPLPDDVEFFHFDADVTKILDDKFAFAETARSFGLSVPKSFKITDPEQV
LNFDFSQEKRKYILKSIPYDQVRRLNLTKLPCDTPSQTAAFVKTLPISEEKPWIMQEFIPGKEYC
THTTARDGESRMYCCCESSAFQVNYENVDQPEIMQWASHFTKELGKTGQLSFDFIQAEDGTVYAI
ECNPRTHSAITMFYNHPGVADAYLGKEPLAESLQPLSDSKPTYWLYHEVWRLNEIRSFKQLQTWV
RNIRRGKEAIFEVSDPLPFLMVHHWQIPLLILDNLRRLKGWIRIDFNMGELID
Q3M6C5
(SEQ ID NO: 86)
MAQSLPLSSAPATPSLPSQTKIAAIIQNICTLALLLLALPINATIVFISLLVFRPQKVKAANPQT
ILISGGKMTKALQLARSFHAAGHRVVLVETHKYWLTGHRFSQAVDKFYTVPAPQDNPQAYIQALV
DIVKQENIDVYIPVTSPVGSYYDSLAKPELSHYCEVFHFDADITQMLDDKFALTQKARSLGLSVP
KSFKITSPEQVINFDFSGETRKYILKSIPYDSVRRLDLTKLPCATPEETAAFVRSLPITPEKPWI
MQEFIPGKEFCTHSTVRNGELRLHCCCESSAFQVNYENVNNPQITEWVQHFVKELKLTGQISFDF
IQAEDGTVYAIECNPRTHSAITTFYDHPQVAEAYLSQAPTTETIQPLTTSKPTYWTYHEVWRLTG
IRSFTQLQRWLGNIWRGTDAIYQPDDPLPFLMVHHWQIPLLLLNNLRRLKGWTRIDFNIGKLVEL
GGD
A0A1Z4ND62
(SEQ ID NO: 87)
MIDTVSLNKSLAEKGFGRREIGVIGRNLATLGLLLLVLPINLLLTGVGLISRVSLRNPISQKTIL
ISGGKMTKALLIARRFHAAGHRVILIESHKYWLTGHRFSNAVNKFYTVPAPEKNPSAYIQALLDI
IKREKVDLYVPVCSPVASYYDALVKSEMGFLTQVFHCDPEMVKMLDDKFTFAETARKLGLSVPKS
FLITHPHQVINFDFQKETRPYILKSIRYDSVRRLDLTKLPCETPEATERFVRSLPISPENPWIMQ
EFIPGQEYCTHSTVKNGELRMHCTSKSSAFQVNYENIDHPRIQSWVSKFVKELGITGQVSFDFIE
TEDGEVYAIECNPRTHSAITMFYNHPRVADAYLDEGVWEQPIQPLPDSKPTYWLYHEIWRLTGIR
SWKDLQYRWKVLSTGVDAIYSLDDPLPFLMVHHWQIPLLLWQNLLQLRGWVRIDFNIGKLVELGG
D
A0A0D8ZR72
(SEQ ID NO: 88)
MQKMFAIFQNLGTLTLLAIAFPFNCIVVLSALVWNLISQPFQKQVVFNPDAKNILIGGGRMTKTL
QLARSFHAAGHRVILFDIDKNWFSGYRFSNAVAGFYTVPDPIKDLEGYTIALRAIAKQENIDFFV
PVGIFANDYFDSKRQPVLSGCCETFHFDADTMKMLDNKFTFTQKARSLSLSVPKAYLITDPEQVL
KFDFSNEKNKYILKSIVYDPVFRLDLTKLPMESLEKMAIHVRNLPISKDNPWILQEFITGQEYCT
HSTVRNGELTVHCCCESSAFQVNYENVDKPEILQWVSHFVKELQLTGQISFDFIQAEDGTIYAIE
CNPRTHSAITMYYNHPGLADAYLGQKPLAELLQPLPDSKPTYWLYHEVWRLNEIRSLKQLQTWFK
NILRGKDAIFDVNDPLPFLMVHHWHIPLLLLDNLQKLKGWVRIDFNIGKIVQVSD
A0A2T1LWM6
(SEQ ID NO: 89)
MDNLFNSSADSSSLSKGWLRSIQGSSLKTLGTLLLLLLMLPFNLALTLTALVWSWVWPFRKRVIA
SNPKTVMISGGKMTKALQLARSFYMAGHRVILVETHKYWLVGHRYSWAVDRFYTIPDPKQDTEGY
LQGLLDIAQKEQVDLYVPVCSPVASYYDALAKELLAQQCDVFHEDAKTVQQLDDKYQFAQAATNL
GLTVPKSFKITHPQQVLDFDFSKETHPYIIKSIPYDSVNRLNLTKLPCASRQDTEMFVNSLPISE
TKPWVMQEFITGQEYCVHSTVKNGELRVYCCCESSAFQVNYEAVDIPEIKQWVTQFVQGMKLTGQ
MSFDFIRTPTGEVYAIECNPRTHSAITLFYNHPDLAKAYLDPEPFSEPLEPLASARPTYWTYHEF
WRLVTHLSSLQEVAYRLGILFKGKDAIFSWNDPLPFLMVHGWQIPLLLLKSLRQGKDWIRIDFNI
GKLVQMGGD
K9XU47
(SEQ ID NO: 90)
MTQIFFVSGRGSAVLQNLGTLVLLLFLLPFNLIAVAFSAVINIFSGSKQRLTKTDVPKRILITGA
KMTKALQLARSFHQRGHEVYLVETHKYWLSGHRFSRAVKGFFTVPTPEKEPDAYCQRLLEIVQQK
NIDVFIPVSSPIASYYDSLAKKILEPDCEAIHFDPEITAMLDDKYAFCTKAKELGLSAPKVFCFT
SPQQVIDFDFESDGSQYIVKSIPYDSVRRLDLTKLPFEGMESYLRSLPISSEKPWVMQEFIRGQE
YCFHATVRKGKIRLHCCSQSSPFQVNYEQVDNPAIYQWVEKFVRELNLTGQICFDMIQTPDGTVY
PIECNPRLHSAITMFHDHPGVADAYLLDGEQAITPLPDSKPTYWTYHELWRLLQVRSLSELQAWW
HKVSRGTDAILQGDDPLPFLMLHNWQIPLLLLDNLRRLKGWIRIDFNIGKLVELEGD
A0A2Z6D2K3
(SEQ ID NO: 91)
MTQSISLSLPESTTPSTGIKVKIVALFKTLGTLTLLLIALPENVLIVLISLLWGIVRVPFTKNVV
ATHPQTILVSGAKMTKALQLARSFHADGHRVILIEGHKYWLSGHRFSKAVSRFYTVPAPQSDPEG
YIQALIEIVKKEKVDVYVPVCSPVASYYDSLAKPALSEYCEVFHFDADITKMLDDKFAFTEKARS
LGLSVPKSFKITDPQQVINFDFSQETRKYILKSINYDSVRRLNLTKLPCDTPEQTAAFVKSLPIS
PETPWIMQEFIPGKEFCTHSTVRDGELRLHCCCHSSAFQINYENVENPQIQAWIQHFVKSLRLTG
QVSFDFIQAEDGQVYAIECNPRTHSAITMFYNHPGVAEAYFGKTPLAAPLEPLPSSKPTYWTYHE
IWRLTGVRSWKQLQTRLNILLRGTDAIYCLDDPIPFLTLHHWQIPLLLLQNLQQLKAWVKIDFNI
GKLVELGGD
A0A5P8W9G9
(SEQ ID NO: 92)
MAQSISLSVPKSTTPSTGVSIKIVALFKTLGTLTLLLIALPINAFIVLLSLLWGILFTKKPAVAA
HPQNILVSGGKMTKALQLARSFHAAGHRVILIEGHKYWLSGHRFSNAVSRFYTVPAPQDDPQGYT
QALLEIVKQEKIDIYVPVCSPVASYYDSLAKPALSEYCEVFHFDADITKMLDDKFAFTDQARSLG
LSVPKSFKITDPEQVINFDFSKETRKYILKSISYDSVRRLNLTKLPCDTPEETAAFVNSLPISPE
KPWIMQEFIPGKELCTHSTVRDGELRLHCCSDSSAFQINYENVENPQIREWVQHFVKSLGLTGQV
SFDFIQAEDGTAYAIECNPRTHSAITMFYNHPGVAEAYFGKTPLAAPLEPLADSKPTYWVYHEIW
RLTGIRSGKQLQTWFARLVRGTDAIYKIDDPLPFLTLHHWQIALLLLQNLQQLKGWVKIDFNIGK
LVELGGD
A0A1S6LXZ0
(SEQ ID NO: 93)
MRKHIFVVFQNLGTLVLLAIAFPLNCIVVLTSLLWSFIKQPFNKSIVVNPNSKNILIAGARMTKT
LQLARSFHAAGHRVIIIDIEKYWLSGNKYSNSVAGFYTVPDPSKDLEGYVETLHAIANTEKIDFF
IPVAIFSVIHYDQGKPPLPDCVEFFHFDADVTKILDDKFAFAETARSFGLSVPKSFKITDPEQVL
NFDFSQEKRKYILKSIPYDQVRRLNLTKLPCDTKSETAAFVKSLPISEENPWIMQEFIPGKEYCT
HTTARDGESRMYCCCESSAFQVNYENVDQREIMQWASHFTKELGKTGQLSFDFIQAEDGTVYAIE
CNPRTHSAITMFYNHPGVADAYLGKEPLAESLQPLPDSKPTYWLYHEVWRLNEIRSFKQLQTWVR
NIRRGKEAIFEVSDPLPFLMVHHWQIPLLILDNLRRLKGWIRIDFNMGELIE
A0A1Z4LFB5
(SEQ ID NO: 94)
MAQSISVSSSPAIPSFPSETKIAVIIQNLLTLALLLLALPINAAIVLVTLLWHTISRPFQQPATK
AANPKNILISGGKMTKALQLARSCAAAGHRVILIETHKYWLSGHRFSQAVDKFYTVPAPQENPER
YTQALIDIIKQENIDVYIPVTSPLGSYYDSLAKPLLSEYCEVFHFDIDITEKLDDKFAFAETARS
LGLSVPKSFKITSAEQVLNFDFSQESRKYILKSIPYDSVRRLDLTKLPCATPEETAAFVRSLPIS
PDKPWIMQEFIPGKEFCTHSTVRDGELRLHCCCESSAFQVNYENVENSQIREWVRHFVKELKLTG
QVSFDFIQAEDGRVYAIECNPRTHSAITTFYDHPQVAQAYLDNEPMAETLQPLPSSKPTYWTYHE
VWRLTGIRSFTQLKKWIANIWRGTDAIYKPDDPLPFLMVHHWQIPLLLLKNLRQIKGWTRIDFNI
GKLVELGGD
A0A4D9CF37
(SEQ ID NO: 95)
MTQSISVASVGQTTQSVTLGLRISALFKNLATLALLLLVLPINAAIVLVSLLLGSQSQAIATEPK
NILISGGKMTKALQLARSFHAAGHRVVLVETHKYWLTGHRFSKAVSRFYTLPTPQSDPEAYTQAL
LDIVQKESINVYVPVCSPVSSYYDSLAKPVLSKYCEVFHCDADVTQMLDDKYAFAEKARSLGLSV
PKSFKITDPKQVINFDFSQEKRKYILKSIPYDSVRRLDLTKLPCESPEATADFVNSLPISSQKPW
IMQEFIPGKEFCTHSTVRNGELRMHCCCESSAFQVNYENVDHPQILEWVRHFVKALGITGQVSFD
FIEAQDGTIYAIECNPRTHSAITMFYNHPDVANAYLSEIPQVEPIQPLINSKPTYWTYHEIWRLT
GIRSFSQLQTWVKNFFGGKDAIYSLSDPLPFLTVHHWQIPLLLLQNLQQLKGWIRIDFNIGKLVE
FGGD
A0A1B2CWF7
(SEQ ID NO: 96)
MAQSIPFDSASPTPQVSWGVRISALWKTVGTLLLLFLALPVNASIVLISLLWGIFSKPFEKRVVA
AAPKNILISGGKMTKALQLARSFHAAGHRVVLVESHKYWLTGHQFSNAVSVFYTVSPPEKDPEGY
TQQLLDIVKKERIDVYVPVCSPVASYYDSLVKPALSQHCEVFHCDAEITQMLDDKYAFSEKARSF
GLSVPKSFKITNPEQVINFDFSQEKRKYILKSIPYDSVRRLNLTKLPCDTPEETAAFVRSLPISP
EKPWIMQEFIPGKEFCTHSTVRNGELRLHCCCESSAFQVNYENVNNPQILEWVKHFIKEMGITGQ
VSFDFIQTEDGTVYAIECNPRTHSAITMFYNHPGVADAYLGKIPLPEPLQPLADSKPTYWLYHEI
WRLTGIRSLSQFWTWLKNLMRGKDAIYQLNDPLPFLTVPHWQITLLLLQNLRQLRGWVKIDFNIG
KLVELGGD
A0A0C1N3Z4
(SEQ ID NO: 97)
MTQSISFSSPVPATPPFCVKTRFIALFQNLGALTLLLLALPINVAIVLISLIWSFLSRLFSTQET
TVAGAKNILISGGKMTKALQLARFFSAAGHRVVLIETHKYWLSGHRFSNAVSRFYTTPTPQDEPE
EYIQTLVDIVKRENIDVYVPVTSPVASYYDSLAKPALSPYCEVLHFDADVTKMLDDKFAFSEKAR
ALGLSVPKSFKITNPEQVLNFDFSQETRKYILKSLPYDSVRRLDLTKLPCNTPEETAAFVKSLPI
SLEKPWIMQEFIPGKEFCTHSTVRNGDLKLHCCSESSAFQVNYENVKNPKIQEWVRHFVKGLGLT
GQVSFDFIQADDGKVYAIECNPRTHSAITMFYNHPQVADAYLGTEPLAEPLAPVPNSKPTYWLYH
EVWRLTGIRSFAQLQSWIRNILRGTDAIYELHDPLPFLMVHHWQIALLLLNNLRQLKGWTKIDFN
IGKLVELGGD
A0A2L2N6B5
(SEQ ID NO: 98)
MRKHIFVVFQNLGTLVLLALAFPLNSIVVLTSLLWNFLKQPFSKSIVVNPNSKNILIAGARMTKT
LQLARSFHAAGHRVIIIDIEKFWSSGNKYSNSVAGFYTVPDPSKDLEGYVETLHAIAKTEKIDFF
IPVAIFSVIHYDRGKPPLPDFCEFFHFDADVTKSLDDKFAFAETARSFGLSVPKSFKITNPEQVL
NFDFSQEKRKYILKSIPYDQIRRLNLTKLPCDTQSETAAFVKSLPISEENPWIMQEFIPGKEYCT
HTTARDGESRMYCCCESSAFQVNYENVDRLEIMEWASHFTKQLGKTGQLSFDFIQAEDGTVYAIE
CNPRTHSAITMFYNHPGVADAYLGKNPLAESLQPLGDSKPTYWLYHEVWRLNEIRSFKQLQTWLR
NIRRGKEAMFEVSDPLPFLMVHHWQIPLLILDNLRRLKGWIRIDENMGELIE
A0A1Z4Q915
(SEQ ID NO: 99)
MVELQFIKARIFAVFRNLGTLALLAIAFPFNCIVVLAALLWNFFTRPFQKQVVLSENPKNILIGG
GRMTKTLQLARSFHAAGHRVILVDIHKYWLSGHRFSKAVAGYYTVPEPQKDLEGYTQALRAIAKK
ENIDFFIPVAIFAVSYFDPQNKPVLAGCCEIFHFDGEVTKMLDDKFAFAEKARSFGLSVPKSFKI
TAPEQVLNFDFSQEKNKYILKSIPYDSVRRLNMTKLPCDTTEQTAAFVKSLPISEENPWIMQEFI
PGQEYCTHSSLRNGELRLHCCCESSAFQVNYENVDKPEIMQWVSHFVKELGLTGEASFDIIQAVD
GTVYPIECNPRTHSAITMFYNHPGVADAYLGKEPLAEPLQPLPDSKPTHWLYHEVWRLTGIRSLK
QLQTWVRNILRGKDAIFEVHDPLPFLMVHHWQIPLLLLDNLRRLKGWIRIDFNLGELIE
A0A2Z5VN68
(SEQ ID NO: 100)
MHFNCGAEKLMAQSISLSLPKSTTPSTGVRIKIVALFKTLGTLTLLLIALPINAFIVLLSLLWSI
PFTKKPAVAAHPQNILVSGGKMTKALQLARSFHAAGHRVILVEGHKYWLSGHRFSKAVSRFYTVP
APQDDPEGYTQALLEIVKQEKIDIYVPVCSPIASYYDSLAKPALSEYCEVFHFDADITKMLDDKF
AFTDQARSLGLSVPKSFKITDPEQVINFDFSKETRKYILKSISYDSVRRLNLTKLPCDTPEETAA
FVNSLPISPEKPWIMQEFIPGKELCTHSTVRDGELRLHCCSDSSAFQINYENVENPQIREWVQHF
VKSLGLTGQVSFDFIQAEDGTAYAIECNPRTHSAITMFYNHPSVAEAYFGKTPLAAPLEPLADSK
PTYWVYHEIWRLTGIRSGKQLQTWFTRLVRGTDAIYKIDDPLPFLTLHHWQIALLLLQNLQQLKG
WVKIDFNIGKLVELGGD
A0A1Z4UKN2
(SEQ ID NO: 101)
MFPINLTLVITAFLTNLITLPFQKKITYENPKNILLTGGKMTKSLQLARSFHRAGHKVFMVETHK
YWLSGHQYSKAVKKFLTVPAPEKDPEGYCQSLLDIVKREKIDVFIPVSSPVASYYDSLAKPILSP
YCEVFHFDTEMTKTLDDKFSLCEQARVLGLTAPKVFLITSPGEIINFDFSQEQNPYIIKSIQYDS
VTRLDMTKFPFEGMKEYVKKLPISKERPWVMQEFIKGQEYCTHSTVRDGEIRLHCCSKSSPFQVN
YEQVDNPEIFQWVQKFVKELNLTGQISFDFMQTEDGKVYPIECNPRTHTAITMFYDHPGLADAYL
EPGKNQPHIEPLPTSKPTYWLYHELWRITGIRSFNDLTNWLNKVIKGKDAMLDKDDPLPFLMVHH
WQIVLLLLQNMVKLKGWVRIDFNIGKLVEIGGD
A0A5Q0GJK5
(SEQ ID NO: 102)
MAQSLPLSSAPATPSLPSQTKIAAIIQNICTLALLLLALPINATIVFISLLVFRPQKVKAANPQT
ILISGGKMTKALQLARSFHAAGHRVVLVETHKYWLTGHRFSQAVDKFYTVPAPQDNPQAYIQALV
DIVKQENIDVYIPVTSPVGSYYDSLAKPELSHYCEVFHFDADITQMLDDKFALTQKARSLGLSVP
KSFKITSPEQVINFDFSGETRKYILKSIPYDSVRRLDLTKLPCATPEETAAFVRSLPITPEKPWI
MQEFIPGKEFCTHSTVRNGELRLHCCCESSAFQVNYENVNNPQITEWVQHFVKELKLTGQISFDF
IQAEDGTVYAIECNPRTHSAITTFYDHPQVAEAYLSQAPTTETIQPLTTSKPTYWTYHEVWRLTG
IRSFTQLQRWLGNIWRGTDAIYQPDDPLPFLMVHHWQIPLLLLNNLRRLKGWTRIDFNIGKLVEL
GGD
A0ZIV3
(SEQ ID NO: 103)
MAQSISLSLGNSPTSSTGVWVKLVALFKTLGTLTLLLIALPFNALIVLISLLWGFVRSPFRQKAV
VAEHPQTILVSGAKMTKALQLARCFHAAGHRVILIEGHKYWLSGHRFSKAVSGFYTVPAPQLDPE
AYIQALVDIVEKEQVDVYVPVCSPVASYYDSLAKPALSEYCEVFHFDADVTKMLDDKFAFTAQAR
SLGLSVPKSFKITDTQQVINFDFSQETHKYILKNIAYDSVRRLNLTKLPCDTPEETAAFVNSLPI
SEENPWIMQEFIPGKELCTHSTVRDGELRLHCCSDSSAFQINYENVENTQIREWVQHFVKSLALT
GQISFDFIQAESGTVYAIECNPRTHSAITMFYNHPGVAEAYLGKTTLDAPLEPLTNSKPTYWIYH
EIWRLTGIRSWKQLQTAVNTLLRGTDAIFQLNDPVPFLTLHHWQIPLLLLKNLQQLKGWVKIDFN
IGKLVELDGD
A0A3S1ANM2
(SEQ ID NO: 104)
MIIHMAQSISLSSPAKTHAPGISASSLKTLGTLTLLLLALPLNASLVLVALLLKSLRPQNFTTEK
PKNILISGGKMTKALQLARSFHNAGHRVILLEAHKYWLTGHRFSSAVNKFYTVEAPEKDPEGYIQ
SLVDIVEKENIDVYVPVCSPVASYYDSLAKKALPQCEVIHCDAEMTQMLDDKHAFAQTAQSFGLS
VPKSFKITDPEQVINFDFSQEKRKYILKSIPYDSVRRLDLTRLPCDTPEATAAFVRSLPISSEKP
WIMQEFIPGKEYCTHSTVRNGVITLHCCCESSAFQVNYENVDNPKIFEWVSRFVKELGITGQVSF
DFIEAEDGNIYAIECNPRTHSAITMFYNHPGVADAYLGTGNNLAEPIQPKFTSKPTYWTYHEIWR
LFNTRSWSDFVYRFKIIKHGKDAIFSWQDPLPFLMNPHWQIFLLLIQNLQKNRGWIRIDFNIGKL
VELGGD

In some embodiments, the one or more biosynthetic enzymes comprise a demethyl-4-deoxygadusol synthase (MysA), or a homolog thereof. Exemplary MysA enzymes for use in the present invention include, but are not limited to, the amino acid sequence of any one of SEQ ID NOs: 105-111, or an amino acid sequence at least 70%, at least 75%, at least 80%, at least 85%, at least 90%, at least 95%, or at least 99% identical to the amino acid sequence of any one of SEQ ID NOs: 105-111:

A0A2K8WSM2
(SEQ ID NO: 105)
MGNGALAENLKEDDKTVIWRPHEEKYRTSEWYTGSGQITTADEGLSFEVTAVYQLK
SEVKVVKDIFAISNHTLANIYRPRSRCIAVVDQTVAELYGEKIEGYFQAQEIPLELMVI
RAWESDKTPETVHRILAFLGKDGCDVSRNEPVLVIGGGVLSDVAGLACALQHRRTP
YIMIGTTIIAAIDAGPSPRTCTNGTQFKNSIGVYHPPVLTLVDRQFFSTLDMGHIRNGM
AEIIKMAVTDDKELFELLEQYGQELIKTRFATIDASEELEKIADLIIYKALYAYMKHEG
TNMFETYQDRPHAYGHTWSPRFEPAVKLMHGHAVTIGMAFGATLAQELGWLSQEE
CQRIINLSSKLGLSVFHPILEDVQIMVDGQKNMRRKRGDGGLWAPLPTTIGACDYVQ
EVEPELLNQAVVAHKKYCSQLPHEGAGEQMYLSDLGLE
A0A0D5ACA9
(SEQ ID NO: 106)
MSNLQAQVVAGDRSFRVEGYERIEYDLIYVDGVFAIENTELADSYRPYGRALMVVD
EAVHDIYGDRISAYFDHHEIALTVVPVHIAETAKSLETFERIVGEFDAFGLVRTEPVLV
VGGGLTTDVAGFACASYRRNTPYIRIPTTLIGLIDASVSIKVAVNYGKHKNRLGAYH
ASQKVLLDFSFLGTLPEDQVRNGMAELIKISVVGNLEIFEMLEQYGPELLRTRFGHLD
GTAELRSVADKLTYSAIATMLELEAPNLHEIDLDRVIAFGHTWSPTLELTPPAPFFHG
HAINIDMALSTTVAEQRGHLSTADRDRVLGVMSSIGLALDSPYLTPELLSEATASILK
TRDGILRAAVPDPIGTCRFLNDLDAAELADVLTLHKKICLDFPRAGEGLDMFTAPTP
A0A0K1S781
(SEQ ID NO: 107)
MAGIKATFTSTDCAFHIQGYEKIDFSLLYVNGAFKIGNPEIAESYAPFRRCLMVIDQT
VYGLYRQQIDQYFAHYQIDLTVFQVSIKEPEKTLRTFEKIVDAFADFGLVRKEPVLVV
GGGLTTDVAGFACSAYRRKTNYIRVPTSLIGLIDASVAIKVAVNHGKLKNRLGAYHA
SQKVILDFSFLGTLPIDQIRNGMAELIKIAVVGNQEIFELLEEHGAALLHSRFGYLNGT
PELQAVGHRLTYKAIQAMLELEVPNLHELDLDRVIAYGHTWSPTLELTPEPPMLHGH
SVNIDMAFTATIAQLRGYISVEDRNRILGLMSRLGLAIDSPYLTPELLWKATEAITRTR
DGLQRAAAPRPIGQCVFMNDLTRSELDKALAVHRAIAQNYPRQGNGEDMYVRLEP
ALEGAGV
A0A0P4UW20
(SEQ ID NO: 108)
MSSVQAKVEVTDQSFHLEGYEKIEFNLDLIEGLFEVGNSGLADNYRTLGRCLAVVDH
NVDRLYGDQLRSYFEYYEIDLTVFAIEITEPTKTIDTFLKITDAFCDFNLKRKEPVLVIG
GGLVLDVAGFACSAYRRSTNYIRVPSTLIGLIDAGVAIKVAVNHGKLKNRLGAYHPP
KQVILDFSFLKTLPVDQIRNGMAELVKIAVVSNEEVENLLEQHGEELLYNHFGFVGN
DAELKQIGHRVNYESIKTMLELEAPNLHELMLDRVIAYGHTWSPTLELAPQIPLLHG
HAVNIDMAISATIAEKRGYISALDRDRILGLMSRLGLALDHPLMEIDLMWKATQSIM
LTRDGFLRAAMPRPIGTCYFVNDLTREELESAIADHKRLCADYPRAGAGIDAYVGSS
ELIGSAN
A0A1Q8JXW2
(SEQ ID NO: 109)
MSNPQAVLSATDTEFRVESWERIEFTLSYVDGVFAPHNTELADLYRPWGRCLMVIDE
TVHEHYGDPIRSYFDHHDIAVTLVPLTIAETAKSLRTLERIVDAYADFGLLRTEPVLV
VGGGLTTDVTGFACASYKRGTPYVRIPTTLIGLIDASVAMKVAVNHGRHKNRLGAF
HASQQVLLDFSFLATLPEAQVRNGVAEMIKIATVANAGLFDLLEKYGDDLLATRFGH
REGTPELRQIAHRCTYDAIHTMLELEHRNLHELDLDRVIAFGHTWSPTLELAPPTPML
HGHAIAIDMAFSATLAARRGDITTGERDRIHRLFSGLGLSVDSTYLTEQLLIDATASIM
QTRAGKLRAALPRPIGTCHFANDIEHTELIETLAAHKAVVAGLPTSVEGVEMWSSAK
TELTTAPNTEART
A0A347Q3N8
(SEQ ID NO: 110)
MTTNLTATVTATENDFRVRAVEERDYLLTYVDGAFSPESSRIADHHRAHGRCLMIV
DANVHRLHGDRIRAYFEHHGIALTALPLAIDETQKSLRTVERIVDAFGEFGLIRKEPV
LVVGGGLLTDVAGFACAVFRRSTDYVRVPTSLIGLIDASVAIKVAVNHGRTKNRLGA
FHASKEVVLDFSFLGTLPTEQVRNGMAELVKIAVVANAEVFRLLEKYGEDLLHTAFG
TVDGTPQLRETARKVTHEAIGTMLALEAPNLRELDLDRAIAFGHTWSPALELAPETP
YLHGHAISVDMALSCTIAERRGYLATSERDRIFWLLSKVGLSLDSPHLTPELLRAATE
SIVQTRDGLQRAAMPRPIGTCCFVNDLTESELLDGLAAHRELVARYPRGGAGEDVRV
TRSGAA
A0A6J4VHE9
(SEQ ID NO: 111)
MSTVQAKFEATETAFHVEGYEKIDFSLVFVNGAFDTKNRELADSYRNFGRCLAVVD
ANVNRLYGSQICEYFKYYNIDLNLFPVTISEPTKNLDTFQSIVDAFADFGLVRKEPVLI
VGGGLVTDVAGFACAAYRRSTNYIRIPTTLISLVDAGIAIKVAVNHGKLKNRLGAYH
APKKVMLDFSFLRTLPTPEVRNGMAELVKIAVVSNVEVFELLCEYGADLLTTHFGFD
GGTPLLKEVAHRINYESIKTMLALETPNLHELDLDRVIAYGHTWSPTLELAPSVPLLH
GHAVNIDMALSATIAEKRGYITVEERDRILGLMSQLGLALDHPLLDIDLLWSATQSIT
LTRDGLQRAAMPRPIGKCFFVNDLTREELDAALAEHKHACAQYPRAGAGVDAYVG
SYQQQNLIEGIANV

In some embodiments, the one or more biosynthetic enzymes comprise an O-methyltransferase (MysB), or a homolog thereof. Exemplary MysB enzymes for use in the present invention include, but are not limited to, the amino acid sequence of SEQ ID NO: 112, or an amino acid sequence at least 70%, at least 75%, at least 80%, at least 85%, at least 90%, at least 95%, or at least 99% identical to the amino acid sequence of SEQ ID NO: 112:

A0A1Z4LFB8
(SEQ ID NO: 112)
MSTTIAKPTARPVTPVGILAKKLEAIVQKINQRTDLPADLVDNITQAWQL
AAGLDPYLEEYTTSESSALTALAEKTSTEAWQEHFSEGTTVRPLEQEMLS
GHVEGQTLKMFVHMTKAKRVLEIGMFTGYSALAMAEALPPDGVLVACEVD
PFAAEVGQAAFDKSPDGKKIRVELGPALETLNKLVEAGESFDMVFIDADK
KEYITYFQTLLDTNLLAPSGFICVDNTLLQGEVYLPTQQRTANGEAIAQF
NRAVALDPRVEQVILPLRDGLTIIRRTA

In some embodiments, the one or more biosynthetic enzymes comprise a non-ribosomal peptide synthetase (NRPS)-like enzyme (MysE), or a homolog thereof. In certain embodiments, the one or more biosynthetic enzymes comprises an enzyme with an amino acid sequence at least 70%, at least 75%, at least 80%, at least 85%, at least 90%, at least 95%, or at least 99% identical to the amino acid sequence of a MysE enzyme, or a homolog thereof.

In some embodiments, the one or more biosynthetic enzymes comprise a mutated D-alanine-D-alanine ligase (MysD), or a homolog thereof. In some embodiments, the mutated MysD is a Y45A mutant. In some embodiments, the mutated MysD is a R223A mutant. In some embodiments, the mutated MysD is a E239A mutant. In some embodiments, the mutated MysD is a Y241A mutant. In some embodiments, the mutated MysD is a Y325A mutant. In some embodiments, the mutated MysD is a K331A mutant. In some embodiments, the mutated MysD is a S332A mutant. In some embodiments, the mutated MysD has improved activity compared to wild type (WT) MysD. In some embodiments, the mutated MysD has improved activity compared to WT MysD when L-Thr is the amino acid substrate. In some embodiments, the mutated MysD has improved activity compared to WT MysD when α-aminobutyric acid (AABA) is the amino acid substrate. In some embodiments, the mutated MysD has at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2.0-fold, at least 2.1-fold, at least 2.2-fold, at least 2.3-fold, at least 2.4-fold, at least 2.5-fold, at least 2.6-fold, at least 2.7-fold, at least 2.8-fold, at least 2.9-fold, at least 3.0-fold, at least 3.1-fold, at least 3.2-fold, at least 3.3-fold, at least 3.4-fold, at least 3.5-fold, at least 3.6-fold, at least 3.7-fold, at least 3.8-fold, at least 3.9-fold, at least 4.0-fold, at least 4.1-fold, at least 4.2-fold, at least 4.3-fold, at least 4.4-fold, at least 4.5-fold, at least 4.6-fold, at least 4.7-fold, at least 4.8-fold, at least 4.9-fold, or at least 5.0-fold improved activity compared to WT MysD.

Compounds of varying structures can be produced using the methods of the present invention. In some embodiments, the compound is a palythine analog. In certain embodiments, the compound has UV-modulating activity. For example, the compounds of the present invention may absorb UV wavelengths between 280 nm and 400 nm. In certain embodiments, the compound is a compound of Formula (I), or a salt thereof:

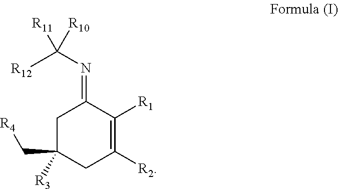

In the compounds of Formula (I) described herein, each of R₁, R₂, R₃, and R₄ may independently be selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl. In some embodiments, R₁ is —OR^{a}, wherein R^{a} is optionally substituted C₁₋₆ alkyl. In certain embodiments, R₁ is —OCH₃. In some embodiments, R₂ is —NH₂. In certain embodiments, R₃ is —OH. In some embodiments, R₄ is —OH. In some embodiments, R₁ is —OCH₃, R₂ is —NH₂, R₃ is —OH, and R₄ is —OH.

The compounds of Formula (I) described herein also include moieties R₁₀, R₁₁, and R₁₂. R₁₀ may be selected from the group consisting of hydrogen and —CO₂R₁₃, wherein R₁₃ may be selected from the group consisting of hydrogen and optionally substituted alkyl; and each of R₁₁ and R₁₂ may be independently selected from the group consisting of hydrogen, optionally substituted alkyl, optionally substituted alkenyl, or R₁₁ and R₁₂ are taken together to form an optionally substituted alkenyl group; provided that if R₁₀ is —CO₂R₁₃ and R₁₁ is hydrogen, R₁₂ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

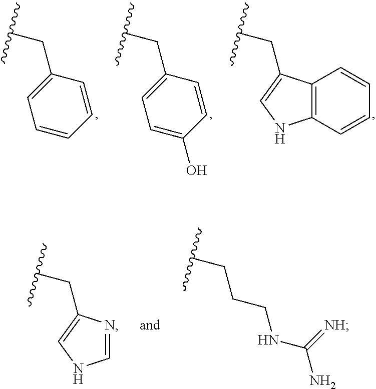 [SMILES: *Cc1ccccc1 ; *Cc1ccc(O)cc1 ; *Cc1c[nH]c2ccccc12 ; *Cc1c[nH]cn1 ; *CCCNC(=N)N]

and provided that if R₁₀ is —CO₂R₁₃ and R₁₂ is hydrogen, R₁₁ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

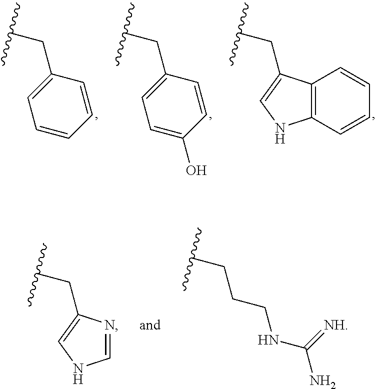 [SMILES: *Cc1ccccc1 ; *Cc1ccc(O)cc1 ; *Cc1c[nH]c2ccccc12 ; *Cc1c[nH]cn1 ; *CCCNC(=N)N]

In certain embodiments, R₁₀ is hydrogen. In certain embodiments, R₁₀ is —CO₂H. In certain embodiments, R₁₁ is hydrogen. In certain embodiments, R₁₁ is optionally substituted alkyl. In certain embodiments, R₁₁ is alkyl optionally substituted with halogen, optionally substituted hydroxyl, optionally substituted amino, or acyl. In certain embodiments, R₁₁ is alkyl optionally substituted with hydroxyl, substituted hydroxyl, amino, substituted amino, or —CO₂H. In certain enbodiments, R₁₂ is hydrogen. In certain embodiments, R₁₂ is optionally substituted alkyl. In certain embodiments, R₁₂ is alkyl optionally substituted with halogen, optionally substituted hydroxyl, optionally substituted amino, or acyl. In certain embodiments, R₁₂ is alkyl optionally substituted with hydroxyl, substituted hydroxyl, amino, substituted amino, or —CO₂H.

In certain embodiments, R₁₀ is hydrogen and R₁₁ is optionally substituted alkyl. In certain embodiments, R₁₀ is hydrogen and R₁₁ is alkyl optionally substituted with halogen, optionally substituted hydroxyl, optionally substituted amino, or acyl. In certain embodiments, R₁₀ is hydrogen and R₁₁ is alkyl optionally substituted with hydroxyl, substituted hydroxyl, amino, substituted amino, or —CO₂H. In certain embodiments, R₁₀ is hydrogen, R₁₂ is hydrogen, and R₁₁ is optionally substituted alkyl. In certain embodiments, R₁₀ is hydrogen, R₁₂ is hydrogen, and R₁₁ is alkyl optionally substituted with halogen, optionally substituted hydroxyl, optionally substituted amino, or acyl. In certain embodiments, R₁₀ is hydrogen, R₁₂ is hydrogen, and R₁₁ is alkyl optionally substituted with hydroxyl, substituted hydroxyl, amino, substituted amino, or —CO₂H.

In certain embodiments, R₁₀ is —CO₂H and R₁₁ is optionally substituted alkyl. In certain embodiments, R₁₀ is —CO₂H and R₁₁ is alkyl optionally substituted with halogen, optionally substituted hydroxyl, optionally substituted amino, or acyl. In certain embodiments, R₁₀ is —CO₂H and R₁₁ is alkyl optionally substituted with hydroxyl, substituted hydroxyl, amino, substituted amino, or —CO₂H. In certain embodiments, R₁₀ is —CO₂H, R₁₂ is hydrogen, and R₁₁ is optionally substituted alkyl. In certain embodiments, R₁₀ is —CO₂H, R₁₂ is hydrogen, and R₁₁ is alkyl optionally substituted with halogen, optionally substituted hydroxyl, optionally substituted amino, or acyl. In certain embodiments, R₁₀ is —CO₂H, R₁₂ is hydrogen, and R₁₁ is alkyl optionally substituted with hydroxyl, substituted hydroxyl, amino, substituted amino, or —CO₂H.

In certain embodiments, R₁₀ is hydrogen and R₁₂ is optionally substituted alkyl. In certain embodiments, R₁₀ is hydrogen and R₁₂ is alkyl optionally substituted with halogen, optionally substituted hydroxyl, optionally substituted amino, or acyl. In certain embodiments, R₁₀ is hydrogen and R₁₂ is alkyl optionally substituted with hydroxyl, substituted hydroxyl, amino, substituted amino, or —CO₂H. In certain embodiments, R₁₀ is hydrogen, R₁₁ is hydrogen, and R₁₂ is optionally substituted alkyl. In certain embodiments, R₁₀ is hydrogen, R₁₁ is hydrogen, and R₁₂ is alkyl optionally substituted with halogen, optionally substituted hydroxyl, optionally substituted amino, or acyl. In certain embodiments, R₁₀ is hydrogen, R₁₁ is hydrogen, and R₁₂ is alkyl optionally substituted with hydroxyl, substituted hydroxyl, amino, substituted amino, or —CO₂H.

In certain embodiments, R₁₀ is —CO₂H and R₁₂ is optionally substituted alkyl. In certain embodiments, R₁₀ is —CO₂H and R₁₂ is alkyl optionally substituted with halogen, optionally substituted hydroxyl, optionally substituted amino, or acyl. In certain embodiments, R₁₀ is —CO₂H and R₁₂ is alkyl optionally substituted with hydroxyl, substituted hydroxyl, amino, substituted amino, or —CO₂H. In certain embodiments, R₁₀ is —CO₂H, R₁₁ is hydrogen, and R₁₂ is optionally substituted alkyl. In certain embodiments, R₁₀ is —CO₂H, R₁₁ is hydrogen, and R₁₂ is alkyl optionally substituted with halogen, optionally substituted hydroxyl, optionally substituted amino, or acyl. In certain embodiments, R₁₀ is —CO₂H, R₁₁ is hydrogen, and R₁₂ is alkyl optionally substituted with hydroxyl, substituted hydroxyl, amino, substituted amino, or —CO₂H.

In certain embodiments, R₁₀ is —CO₂H, R₁₁ is hydrogen, and R₁₂ is

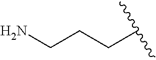 [SMILES: *CCCN]

In certain embodiments, R₁₀ is hydrogen, R₁₁ is hydrogen, and R₁₂ is

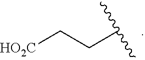 [SMILES: *CCC(=O)O]

In some embodiments, R₁₀ is —CO₂H, and R₁₁ and R₁₂ are taken together to form

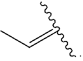 [SMILES: *=CC]

In some embodiments, R₁₀ is —CO₂H, R₁₁ is hydrogen, and R₁₂ is

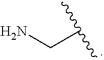 [SMILES: *CN]

In some embodiments, R₁ is —OCH₃, R₂ is —NH₂, R₃ is —OH, R₄ is —OH, R₁₀ is —CO₂H, R₁₁ is hydrogen, and R₁₂ is

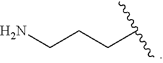 [SMILES: *CCCN]

In some embodiments, R₁ is —OCH₃, R₂ is —NH₂, R₃ is —OH, R₄ is —OH, R₁₀ is hydrogen, R₁₁ is hydrogen, and R₁₂ is

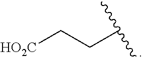 [SMILES: *CCC(=O)O]

In some embodiments, R₁ is —OCH₃, R₂ is —NH₂, R₃ is —OH, R₄ is —OH, R₁₀ is —CO₂H, and R₁₁ and R₁₂ are taken together to form

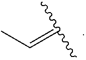 [SMILES: *=CC]

In some embodiments, R₁ is —OCH₃, R₂ is —NH₂, R₃ is —OH, R₄ is —OH, R₁₀ is —CO₂H, R₁₁ is hydrogen, and R₁₂ is

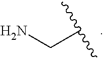 [SMILES: *CN]

In some embodiments, the compound of Formula (I) is of the formula:

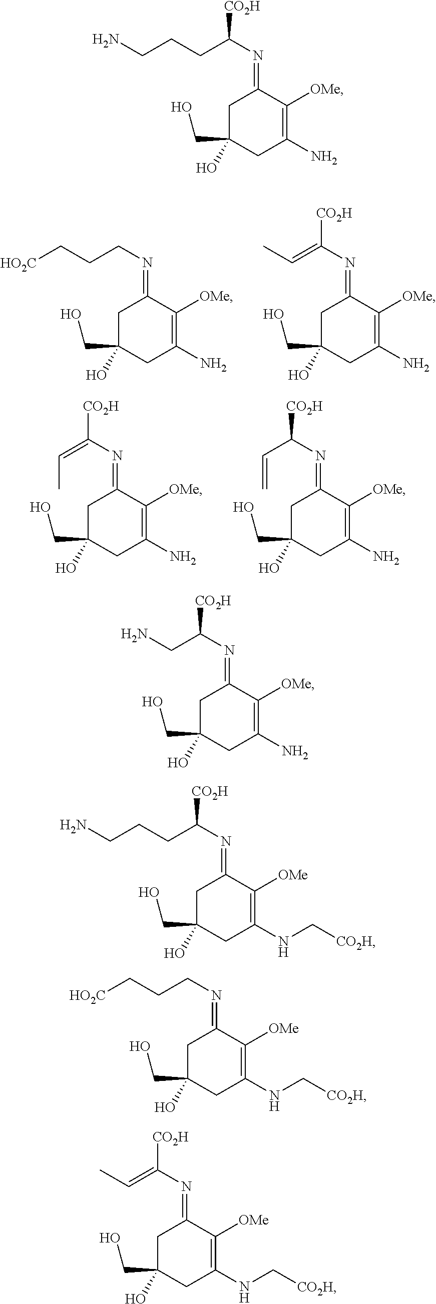

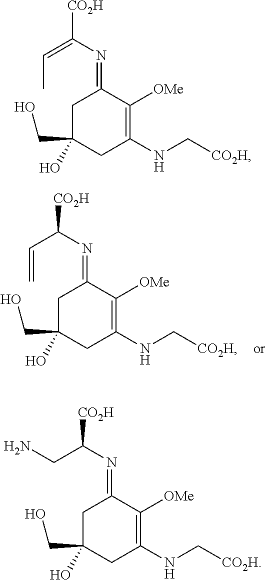

In certain embodiments, the compound of Formula (I) is not.

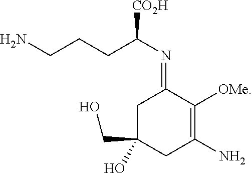 [SMILES: COC1=C(N)C[C@@](O)(CO)C/C1=N\[C@@H](CCCN)C(=O)O]

In certain embodiments, the compound of Formula (I) is not

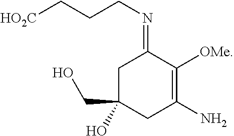 [SMILES: COC1=C(N)C[C@@](O)(CO)C/C1=N\CCCC(=O)O]

In certain embodiments, the compound of Formula (I) is not

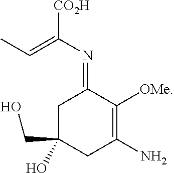 [SMILES: C/C=C(/N=C1\C[C@](O)(CO)CC(N)=C1OC)C(=O)O]

In certain embodiments, the compound of Formula (I) is not

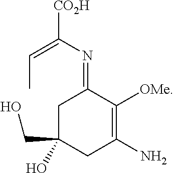 [SMILES: C/C=C(\N=C1/C[C@](O)(CO)CC(N)=C1OC)C(=O)O]

In certain embodiments, the compound of Formula (I) is not

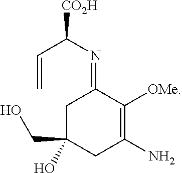 [SMILES: C=C[C@H](/N=C1\C[C@](O)(CO)CC(N)=C1OC)C(=O)O]

In certain embodiments, the compound of Formula (I) is not

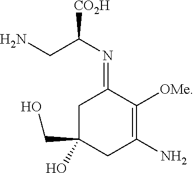 [SMILES: COC1=C(N)C[C@@](O)(CO)C/C1=N\[C@@H](CN)C(=O)O]

In certain embodiments, the compound of Formula (I) is not

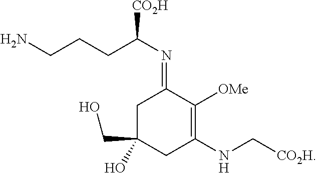 [SMILES: COC1=C(NCC(=O)O)C[C@@](O)(CO)C/C1=N\[C@@H](CCCN)C(=O)O]

In certain embodiments, the compound of Formula (I) is not

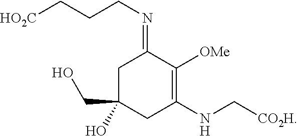 [SMILES: COC1=C(NCC(=O)O)C[C@@](O)(CO)C/C1=N\CCCC(=O)O]

In certain embodiments, the compound of Formula (I) is not

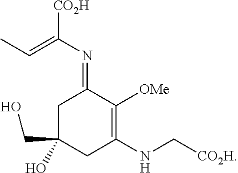 [SMILES: C/C=C(/N=C1\C[C@](O)(CO)CC(NCC(=O)O)=C1OC)C(=O)O]

In certain embodiments, the compound of Formula (I) is not

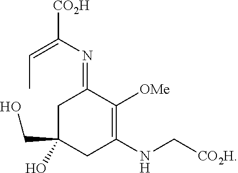 [SMILES: C/C=C(\N=C1/C[C@](O)(CO)CC(NCC(=O)O)=C1OC)C(=O)O]

In certain embodiments, the compound of Formula (I) is not

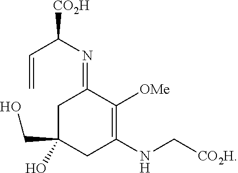 [SMILES: C=C[C@H](/N=C1\C[C@](O)(CO)CC(NCC(=O)O)=C1OC)C(=O)O]

In certain embodiments, the compound of Formula (I) is not

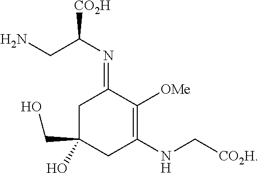 [SMILES: COC1=C(NCC(=O)O)C[C@@](O)(CO)C/C1=N\[C@@H](CN)C(=O)O]

The methods disclosed herein may further comprise providing a substrate of one of the MAA biosynthetic enzymes to the recombinant microorganism. In some embodiments, the substrate is a compound of Formula (II), or a salt thereof:

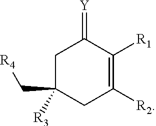

In the compounds of Formula (II) described herein, each of R₁, R₂, R₃, and R₄ may independently be selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl. In certain embodiments, R₁ is —OH. In certain embodiments, R₁ is —OCH₃. In some embodiments, R₂ is —OH. In certain embodiments, R₂ is —NH₂. In some embodiments, R₂ is —(NH)R^{b}, wherein R^{b} is optionally substituted alkyl. In certain embodiments, R₂ is —NHCH₂CO₂H. In some embodiments, R₃ is —OH. In some embodiments, R₄ is —OH. In some embodiments, R₁ is —OCH₃, R₂ is —(NH)R^{b}, R₃ is —OH, and R₄ is —OH. In some embodiments, R₁ is —OCH₃, R₂ is —NH₂, R₃ is —OH, and R₄ is —OH. In some embodiments, R₁ is —OH, R₂ is —OH, R₃ is —OH, and R₄ is —OH. In some embodiments, R₁ is —OCH₃, R₂ is —OH, R₃ is —OH, and R₄ is —OH.

The compounds of Formula (II) described herein also include a moiety Y. Y may be O or

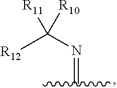

wherein R₁₀ may be selected from the group consisting of hydrogen and —CO₂R₁₃, wherein R₁₃ may be selected from the group consisting of hydrogen and optionally substituted alkyl; and each of R₁₁ and R₁₂ may be independently selected from the group consisting of hydrogen, optionally substituted alkyl, optionally substituted alkenyl, or R₁₁ and R₁₂ are taken together to form an optionally substituted alkenyl group; provided that if R₁₀ is —CO₂R₁₃ and R₁₁ is hydrogen, R₁₂ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

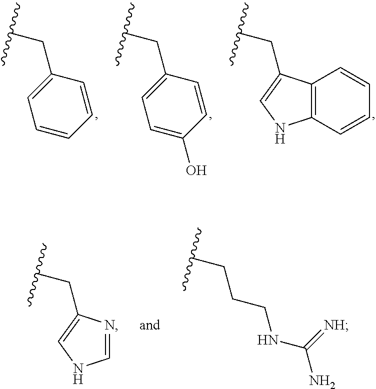 [SMILES: *Cc1ccccc1 ; *Cc1ccc(O)cc1 ; *Cc1c[nH]c2ccccc12 ; *Cc1c[nH]cn1 ; *CCCNC(=N)N]

and provided that if R₁₀ is —CO₂R₁₃ and R₁₂ is hydrogen, R₁₁ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

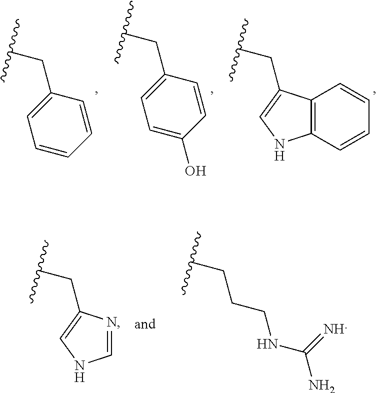 [SMILES: *Cc1ccccc1 ; *Cc1ccc(O)cc1 ; *Cc1c[nH]c2ccccc12 ; *Cc1c[nH]cn1 ; *CCCNC(=N)N]

In certain embodiments, Y is O. In some embodiments, Y is

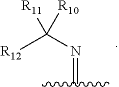

In certain embodiments, Y is

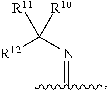

R₁₀ is —CO₂H, R₁₁ is hydrogen, and R₁₂ is

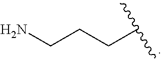 [SMILES: *CCCN]

In certain embodiments, Y is

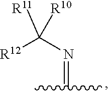

R₁₀ is hydrogen, R₁₁ is hydrogen, and R₁₂ is

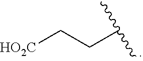 [SMILES: *CCC(=O)O]

In certain embodiments, Y is

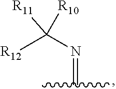

R₁₀ is —CO₂H, and R₁₁ and R₁₂ are taken together to form

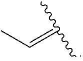 [SMILES: *=CC]

In certain embodiments, Y is

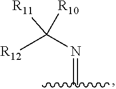

R₁₀ is —CO₂H, R₁₁ is hydrogen, and R₁₂ is

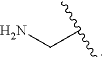 [SMILES: *CN]

In some embodiments, the substrate is a compound of the formula:

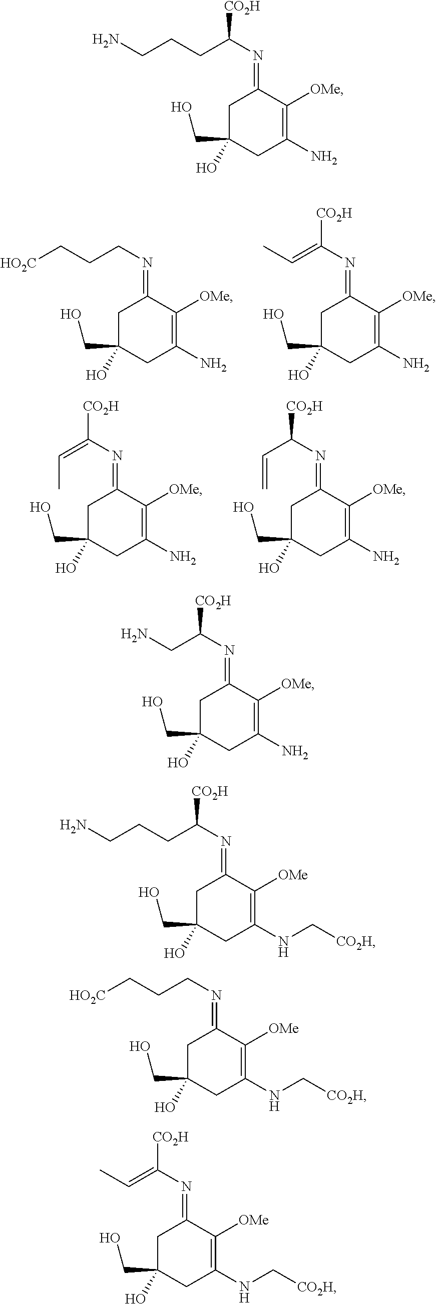

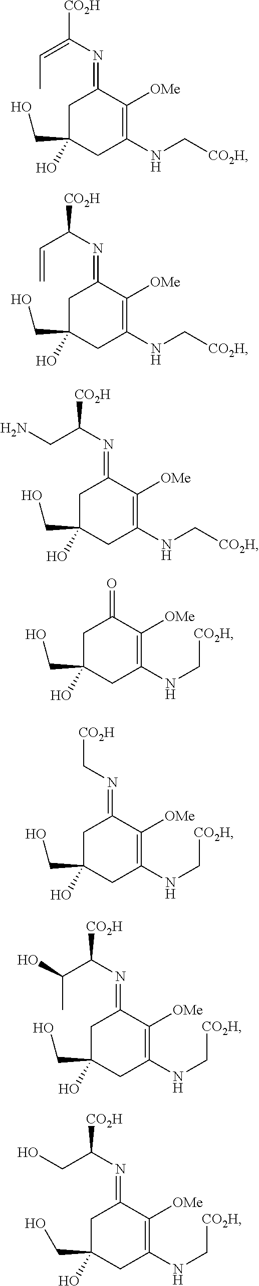

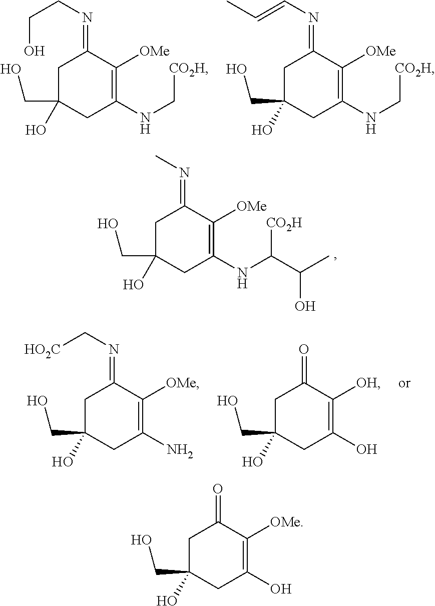

In certain embodiments, the substrate is not a compound of the formula

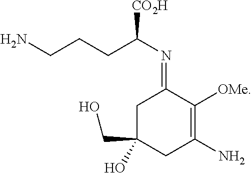 [SMILES: COC1=C(N)C[C@@](O)(CO)C/C1=N\[C@@H](CCCN)C(=O)O]

In certain embodiments, the substrate is not a compound of the formula

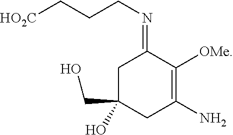 [SMILES: COC1=C(N)C[C@@](O)(CO)C/C1=N\CCCC(=O)O]

In certain embodiments, the substrate is not a compound of the formula

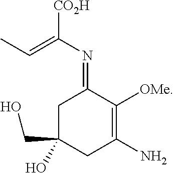 [SMILES: C/C=C(/N=C1\C[C@](O)(CO)CC(N)=C1OC)C(=O)O]

In certain embodiments, the substrate is not a compound of the formula

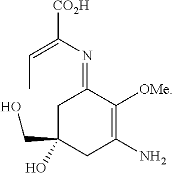 [SMILES: C/C=C(\N=C1/C[C@](O)(CO)CC(N)=C1OC)C(=O)O]

In certain embodiments, the substrate is not a compound of the formula

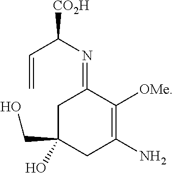 [SMILES: C=C[C@H](/N=C1\C[C@](O)(CO)CC(N)=C1OC)C(=O)O]

In certain embodiments, the substrate is not a compound of the formula

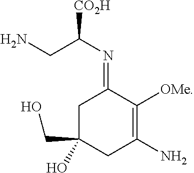 [SMILES: COC1=C(N)C[C@@](O)(CO)C/C1=N\[C@@H](CN)C(=O)O]

In certain embodiments, the substrate is not a compound of the formula

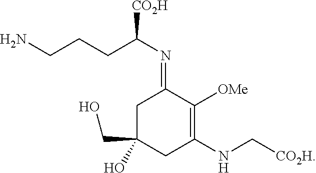 [SMILES: COC1=C(NCC(=O)O)C[C@@](O)(CO)C/C1=N\[C@@H](CCCN)C(=O)O]

In certain embodiments, the substrate is not a compound of the formula

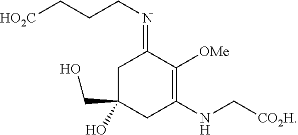 [SMILES: COC1=C(NCC(=O)O)C[C@@](O)(CO)C/C1=N\CCCC(=O)O]

In certain embodiments, the substrate is not a compound of the formula

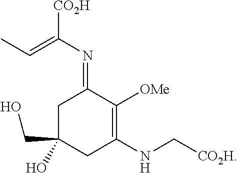 [SMILES: C/C=C(/N=C1\C[C@](O)(CO)CC(NCC(=O)O)=C1OC)C(=O)O]

In certain embodiments, the substrate is not a compound of the formula

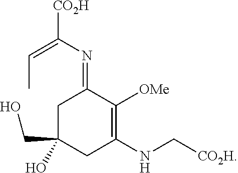 [SMILES: C/C=C(\N=C1/C[C@](O)(CO)CC(NCC(=O)O)=C1OC)C(=O)O]

In certain embodiments, the substrate is not a compound of the formula

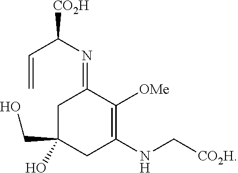 [SMILES: C=C[C@H](/N=C1\C[C@](O)(CO)CC(NCC(=O)O)=C1OC)C(=O)O]

In certain embodiments, the substrate is not a compound of the formula

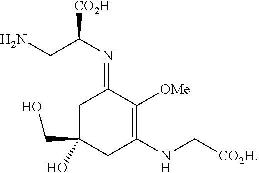 [SMILES: COC1=C(NCC(=O)O)C[C@@](O)(CO)C/C1=N\[C@@H](CN)C(=O)O]

In certain embodiments, the substrate is not a compound of the formula

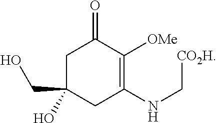 [SMILES: COC1=C(NCC(=O)O)C[C@@](O)(CO)CC1=O]

In certain embodiments, the substrate is not a compound of the formula

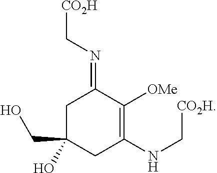 [SMILES: COC1=C(NCC(=O)O)C[C@@](O)(CO)C/C1=N\CC(=O)O]

In certain embodiments, the substrate is not a compound of the formula

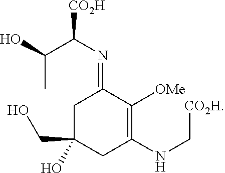 [SMILES: COC1=C(NCC(=O)O)C[C@@](O)(CO)C/C1=N\[C@H](C(=O)O)[C@@H](C)O]

In certain embodiments, the substrate is not a compound of the formula

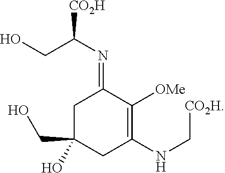 [SMILES: COC1=C(NCC(=O)O)C[C@@](O)(CO)C/C1=N\[C@@H](CO)C(=O)O]

In certain embodiments, the substrate is not a compound of the formula

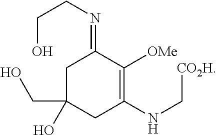 [SMILES: COC1=C(NCC(=O)O)CC(O)(CO)C/C1=N\CCO]

In certain embodiments, the substrate is not a compound of the formula

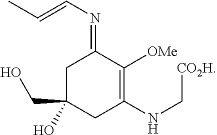 [SMILES: C/C=C/N=C1\C[C@](O)(CO)CC(NCC(=O)O)=C1OC]

In certain embodiments, the substrate is not a compound of the formula

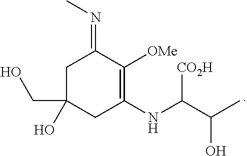 [SMILES: C/N=C1\CC(O)(CO)CC(NC(C(=O)O)C(C)O)=C1OC]

In certain embodiments, the substrate is not a compound of the formula

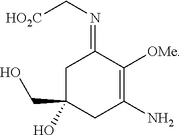 [SMILES: COC1=C(N)C[C@@](O)(CO)C/C1=N\CC(=O)O]

In certain embodiments, the substrate is not a compound of the formula

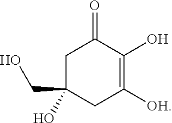 [SMILES: O=C1C[C@](O)(CO)CC(O)=C1O]

In certain embodiments, the substrate is not a compound of the formula

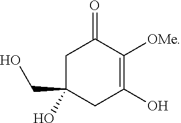 [SMILES: COC1=C(O)C[C@@](O)(CO)CC1=O]

Any suitable microorganism that can be genetically manipulated (e.g., genomically engineered, or transformed with a suitable vector to express a heterologous gene) may be used in the methods of the present invention. For example, the recombinant microorganism may be a species of bacteria or yeast. In some embodiments, the recombinant microorganism is a species of cyanobacteria. In some embodiments, the recombinant microorganism is a species of bacteria from the human microbiome (e.g., including, but not limited to, any of the species listed herein). In certain embodiments, the recombinant microorganism is E. coli.

The present disclosure also encompasses recombinant microorganisms for use in performing the methods of the present invention. For instance, in one aspect the present disclosure includes recombinant microorganisms comprising a heterologous nucleic acid encoding one or more MAA biosynthetic enzymes, wherein the one or more MAA biosynthetic enzymes comprise a D-alanine-D-alanine ligase (MysD), or a homolog thereof. In another aspect, the present disclosure provides methods of producing a compound, comprising culturing such a recombinant microorganism under conditions suitable for production of the compound and isolating the compound from the recombinant microorganism.

### Compositions

In one aspect, the present disclosure provides compositions comprising a compound produced by the methods of the present invention (e.g., a compound of Formula (I), or a salt thereof). In some embodiments, the composition optionally comprises one or more suitable excipients. In certain embodiments, the compositions described herein comprise a compound of Formula (I), or a salt thereof, and an excipient.

In certain embodiments, the compound described herein is provided in an effective amount in the composition. In certain embodiments, the effective amount is a therapeutically effective amount. In certain embodiments, the effective amount is a prophylactically effective amount. In certain embodiments, the compound is provided in an amount effective for preventing sunburn in a subject. In certain embodiments, the compound is provided in an amount effective for preventing cancer (e.g., skin cancer) in the subject. In certain embodiments, the compound is provided in an amount effective for treating or preventing a chronic inflammatory disease or condition in a subject in need thereof. In certain embodiments, the effective amount is an amount effective for reducing symptoms (e.g., symptoms of sunburn) by at least about 10%, at least about 20%, at least about 30%, at least about 40%, at least about 50%, at least about 60%, at least about 70%, at least about 80%, at least about 90%, at least about 95%, or at least about 98%.

Compositions described herein can be prepared by any method known in the art. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit, or into a formulation for topical administration.

Relative amounts of the active ingredient, the excipient, and/or any additional ingredients in a composition described herein will vary. The composition may comprise between 0.1% and 100% (w/w) active ingredient.

Excipients used in the manufacture of the provided compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Dosage forms for topical and/or transdermal administration of a compound produced by the methods described herein may include ointments, pastes, creams, lotions, gels, powders, solutions, sprays, inhalants, and/or patches. Generally, the active ingredient is admixed under sterile conditions with an acceptable carrier or excipient and/or any needed preservatives and/or buffers as can be required. In some embodiments, the composition for topical administration is formulated as a sunscreen. In certain embodiments, the composition for topical administration is formulated as a cosmetic.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

The compositions described herein may also comprise one or more additional active ingredients (e.g., additional compounds with UV-modulating, anti-inflammatory, and/or anti-oxidative activity). In certain embodiments, a composition described herein including a compound described herein and an additional active ingredient shows a synergistic effect (e.g., improved prevention of sunburn in a subject) that is absent in a composition including either the compound or the additional active ingredient, but not both.

Thus, in one aspect, the present disclosure contemplates compositions comprising a compound produced by any of the methods of the present invention and optionally an excipient. In some embodiments, the composition is for topical administration. In certain embodiments, the composition is formulated as a sunscreen. In certain embodiments, the composition is formulated as a cosmetic (e.g., make-up, concealer, a moisturizer, etc.). In another aspect, the present disclosure provides methods of making a composition as described herein, comprising culturing a recombinant microorganism under conditions suitable for production of a compound, as described herein, and isolating the compound from the recombinant microorganism, wherein the recombinant microorganism comprises a heterologous nucleic acid encoding one or more MAA biosynthetic enzymes, wherein the one or more MAA biosynthetic enzymes comprise a D-alanine-D-alanine ligase (MysD), or a homolog thereof, and adding the compound to one or more excipients to produce the composition.

### Methods of Prevention and Treatment

In another aspect, the present disclosure includes methods of administering a compound (e.g., any of the compounds disclosed herein). In some embodiments, a method of administering a compound comprises applying any of the compositions disclosed herein to a subject. In certain embodiments, the composition is applied on the skin of a subject in need thereof. In some embodiments, the method is a method preventing sunburn in a subject in need thereof.

In certain embodiments, the method is a method of preventing cancer in a subject in need thereof (e.g., skin cancers such as melanoma, basal cell carcinoma, or squamous cell carcinoma as described herein). MAAs and related compounds have utility as anti-cancer agents through their antioxidant and anti-proliferative activities (Mar. Drugs 2017, 15(10), 326). For example, the compounds of the present disclosure have UV-modulating activity and may prevent DNA damage in skin cells caused by UV radiation from the sun when applied to the skin in any of the compositions disclosed herein.

In certain embodiments, the method is a method of preventing or treating a chronic inflammatory disease in a subject in need thereof. For example, compounds of the present disclosure have anti-oxidative and anti-inflammatory activities and may prevent or alleviate symptoms of an inflammatory disease when applied to the skin in any of the compositions disclosed herein.

### Compounds

In another aspect, the present disclosure provides compounds produced by the methods of the present invention. In some embodiments, the present disclosure provides compounds produced by culturing a recombinant microorganism under conditions suitable for production of the compound and isolating the compound from the recombinant microorganism. In certain embodiments, the recombinant microorganism comprises a heterologous nucleic acid encoding one or more MAA biosynthetic enzymes, wherein the one or more MAA biosynthetic enzymes comprise a D-alanine-D-alanine ligase (MysD), or a homolog thereof. In some embodiments, the heterologous nucleic acid encodes additional MAA biosynthetic enzymes (e.g., MysA, MysB, MysC, MysE, and/or MysH, or homologs or variants thereof).

In some embodiments, the compound is a compound of Formula (I), or a salt thereof:

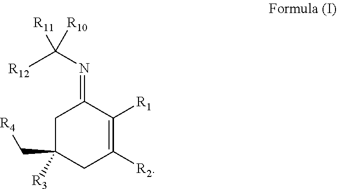

In the compounds of Formula (I) described herein, each of R₁, R₂, R₃, and R₄ may independently be selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl. In some embodiments, R₁ is —OR^{aa}, wherein R^{a} is optionally substituted C₁₋₆ alkyl. In certain embodiments, R₁ is —OCH₃. In some embodiments, R₂ is —NH₂. In certain embodiments, R₃ is —OH. In some embodiments, R₄ is —OH.

The compounds of Formula (I) described herein also include moieties R₁₀, R₁₁, and R₁₂. R₁₀ may be selected from the group consisting of hydrogen and —CO₂R₁₃, wherein R₁₃ may be selected from the group consisting of hydrogen and optionally substituted alkyl; and each of R₁₁ and R₁₂ may be independently selected from the group consisting of hydrogen, optionally substituted alkyl, optionally substituted alkenyl, or R₁₁ and R₁₂ are taken together to form an optionally substituted alkenyl group; provided that if R₁₀ is —CO₂R₁₃ and R₁₁ is hydrogen, R₁₂ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

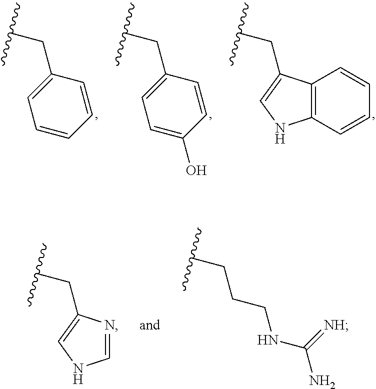 [SMILES: *Cc1ccccc1 ; *Cc1ccc(O)cc1 ; *Cc1c[nH]c2ccccc12 ; *Cc1c[nH]cn1 ; *CCCNC(=N)N]

and provided that if R₁₀ is —CO₂R₁₃ and R₁₂ is hydrogen, R₁₁ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

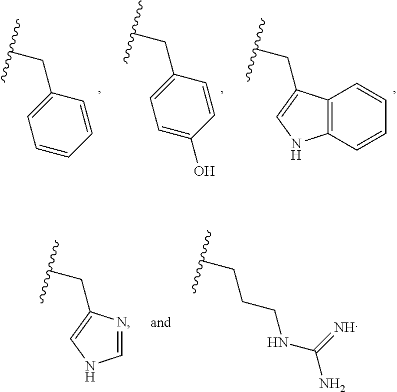 [SMILES: *Cc1ccccc1 ; *Cc1ccc(O)cc1 ; *Cc1c[nH]c2ccccc12 ; *Cc1c[nH]cn1 ; *CCCNC(=N)N]

In certain embodiments, R₁₀ is —CO₂H, R₁₁ is hydrogen, and R₁₂ is

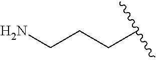 [SMILES: *CCCN]

In certain embodiments, R₁₀ is hydrogen, R₁₁ is hydrogen, and R₁₂ is

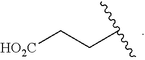 [SMILES: *CCC(=O)O]

In some embodiments, R₁₀ is —CO₂H, and R₁₁ and R₁₂ are taken together to form

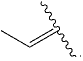 [SMILES: *=CC]

In some embodiments, R₁₀ is —CO₂H, R₁₁ is hydrogen, and R₁₂ is

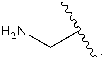 [SMILES: *CN]

In some embodiments, R₁ is —OCH₃, R₂ is —NH₂, R₃ is —OH, R₄ is —OH, R₁₀ is —CO₂H, R₁₁ is hydrogen, and R₁₂ is

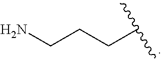 [SMILES: *CCCN]

In some embodiments, R₁ is —OCH₃, R₂ is —NH₂, R₃ is —OH, R₄ is —OH, R₁₀ is hydrogen, R₁₁ is hydrogen, and R₁₂ is

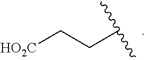 [SMILES: *CCC(=O)O]

In some embodiments, R₁ is —OCH₃, R₂ is —NH₂, R₃ is —OH, R₄ is —OH, R₁₀ is —CO₂H, and R₁₁ and R₁₂ are taken together to form

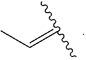 [SMILES: *=CC]

In some embodiments, R₁ is —OCH₃, R₂ is —NH₂, R₃ is —OH, R₄ is —OH, R₁₀ is —CO₂H, R₁₁ is hydrogen, and R₁₂ is

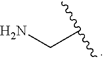 [SMILES: *CN]

In some embodiments, the compound of Formula (I) is of the formula:

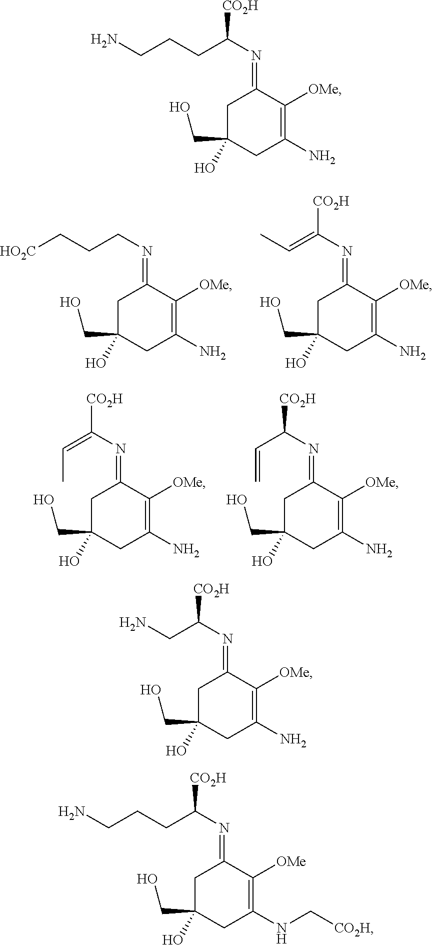

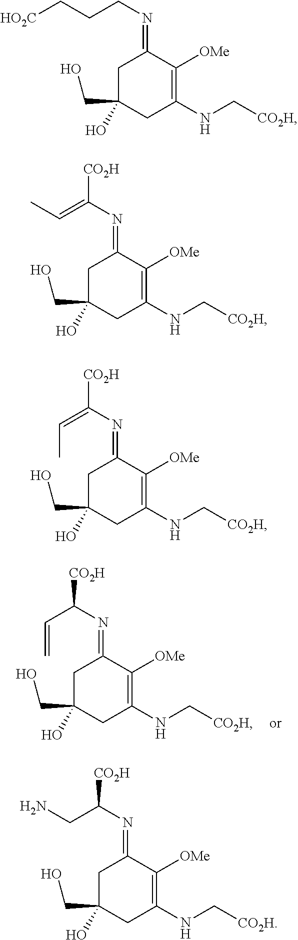

In certain embodiments, the compound of Formula (I) is not

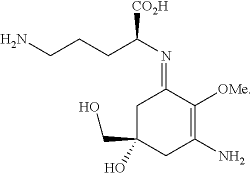 [SMILES: COC1=C(N)C[C@@](O)(CO)C/C1=N\[C@@H](CCCN)C(=O)O]

In certain embodiments, the compound of Formula (I) is not

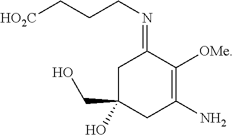 [SMILES: COC1=C(N)C[C@@](O)(CO)C/C1=N\CCCC(=O)O]

In certain embodiments, the compound of Formula (I) is not

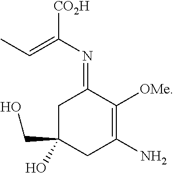 [SMILES: C/C=C(/N=C1\C[C@](O)(CO)CC(N)=C1OC)C(=O)O]

In certain embodiments, the compound of Formula (I) is not

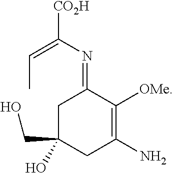 [SMILES: C/C=C(\N=C1/C[C@](O)(CO)CC(N)=C1OC)C(=O)O]

In certain embodiments, the compound of Formula (I) is not

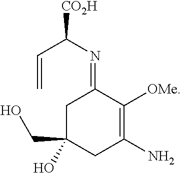 [SMILES: C=C[C@H](/N=C1\C[C@](O)(CO)CC(N)=C1OC)C(=O)O]

In certain embodiments, the compound of Formula (I) is not

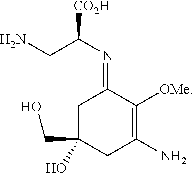 [SMILES: COC1=C(N)C[C@@](O)(CO)C/C1=N\[C@@H](CN)C(=O)O]

In certain embodiments, the compound of Formula (I) is not

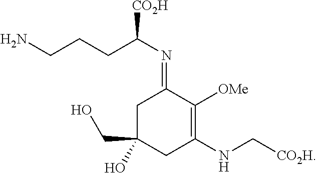 [SMILES: COC1=C(NCC(=O)O)C[C@@](O)(CO)C/C1=N\[C@@H](CCCN)C(=O)O]

In certain embodiments, the compound of Formula (I) is not

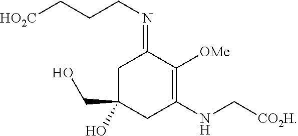 [SMILES: COC1=C(NCC(=O)O)C[C@@](O)(CO)C/C1=N\CCCC(=O)O]

In certain embodiments, the compound of Formula (I) is not

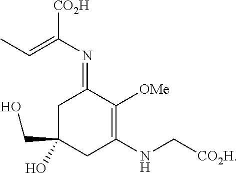 [SMILES: C/C=C(/N=C1\C[C@](O)(CO)CC(NCC(=O)O)=C1OC)C(=O)O]

In certain embodiments, the compound of Formula (I) is not

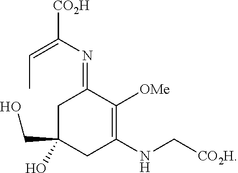 [SMILES: C/C=C(\N=C1/C[C@](O)(CO)CC(NCC(=O)O)=C1OC)C(=O)O]

In certain embodiments, the compound of Formula (I) is not

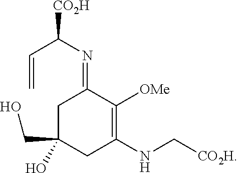 [SMILES: C=C[C@H](/N=C1\C[C@](O)(CO)CC(NCC(=O)O)=C1OC)C(=O)O]

In certain embodiments, the compound of Formula (I) is not

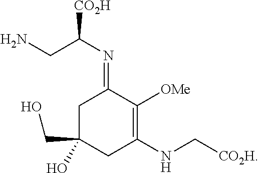 [SMILES: COC1=C(NCC(=O)O)C[C@@](O)(CO)C/C1=N\[C@@H](CN)C(=O)O]

In some embodiments, a compound of the present invention, or a salt thereof, is provided in a composition (e.g., in any of the forms disclosed herein). In some embodiments, the composition is for topical administration. In certain embodiments, the composition is formulated as a sunscreen. In certain embodiments, the composition is formulated as a cosmetic.

In one aspect, the present disclosure provides methods of administering the compounds of the present invention comprising applying any of the compositions disclosed herein to a subject. In some embodiments, the composition is applied on the skin of a subject. In certain embodiments, the composition is applied on the skin of a subject in need thereof as a method of preventing sunburn (e.g., when the composition is formulated as a sunscreen). In certain embodiments, the composition is applied on the skin of a subject in need thereof as a method of preventing cancer. In certain embodiments, the composition is applied on the skin of a subject in need thereof as a method of treating or preventing a chronic inflammatory disease.

### Examples

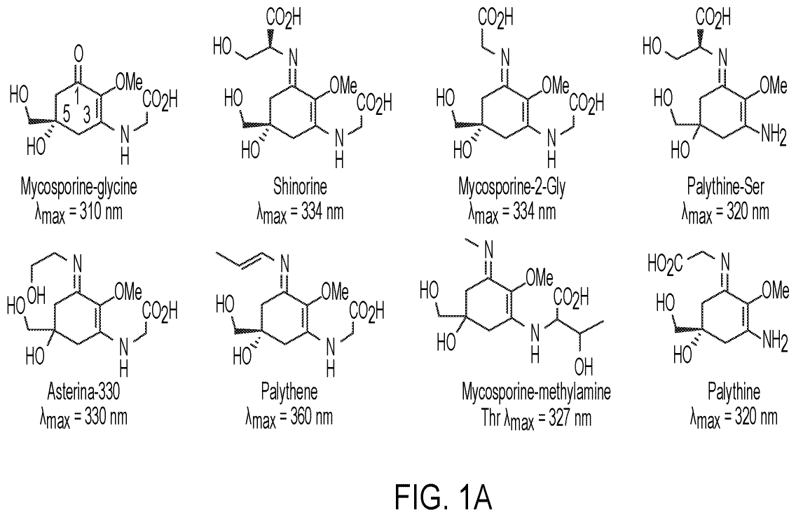

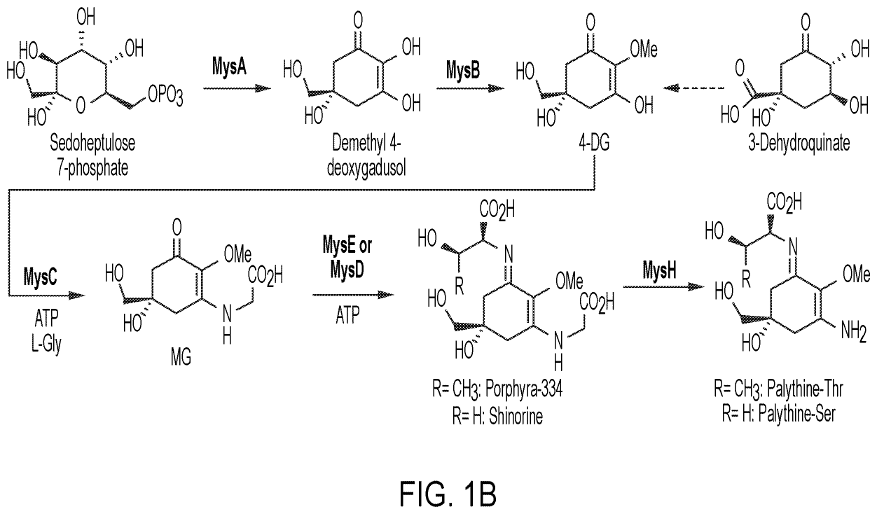

Mycosporine-like amino acids (MAAs) are a family of natural, thermally and photochemically stable UV protectants (FIG. 1A).¹⁶ Originally isolated from terrestrial fungal species, over 30 MAA analogs have been identified from taxonomically diverse marine and terrestrial organisms (e.g., cyanobacteria, eukaryotic algae, corals, plants, and vertebrates) and possess various functional groups at the C1 and, to a lesser extent, the C3 of the characteristic cyclohexenimine core (FIG. 1A).¹⁶⁻¹⁸ Indeed, the majority of MAAs carry a C3-L-Gly moiety, though L-Ala, L-Glu, and other amine-containing components also appear. Common amino acid building blocks at the C1 include L-Ser (shinorine), L-Thr (porphyra-334), L-Gly (mycosporine-2-Gly) and L-Ala.¹⁶⁻¹⁸ These moieties at the C1 and C3 can likely be converted into other functional groups, including amino alcohol (e.g., asterina-330), enaminone (e.g., palythene), methyl amine (e.g., mycosporine-methylamine-Thr), or an amine group (e.g., palythine and palythine-Ser),^{17,19} while glycosylated MAAs have been produced in a variety of organisms.^{20,21} Of note, except a few analogs (e.g., mycosporine-glycine, porphyra-334, palythene and palythine),²²⁻²⁵ the absolute configuration of the majority of MAAs, particularly the C5, has not been fully elucidated. Despite notable structural diversity, these MAA analogs display absorption maxima between 280 and 400 nm and possess extinction coefficients of up to 50,000 M⁻¹·cm⁻¹.¹⁶⁻¹⁷ They are among the strongest UV absorbing compounds, and the cyclohexenimine core is critical for the dissemination of UV energy. Furthermore, accumulated evidence demonstrates the antioxidative, anti-inflammatory and antiaging properties of MAAs, providing another mechanism of photoprotection.¹⁴

This study uses unnatural amino acids as the substrates of the MysD reaction.

### Example 1: MysD Accepts Unnatural Amino Acid Substrates.

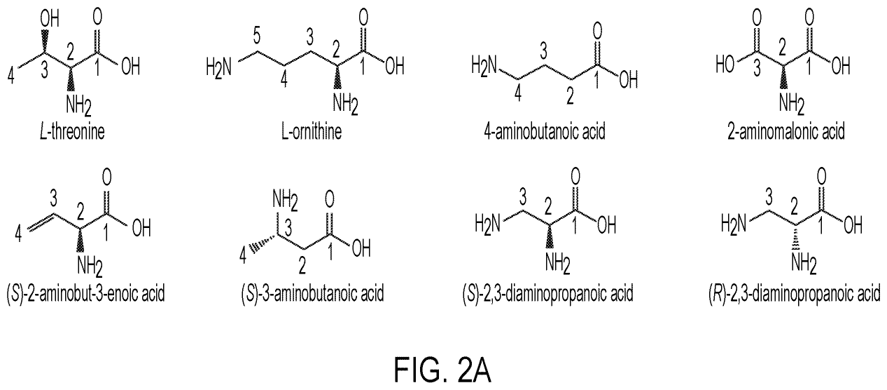

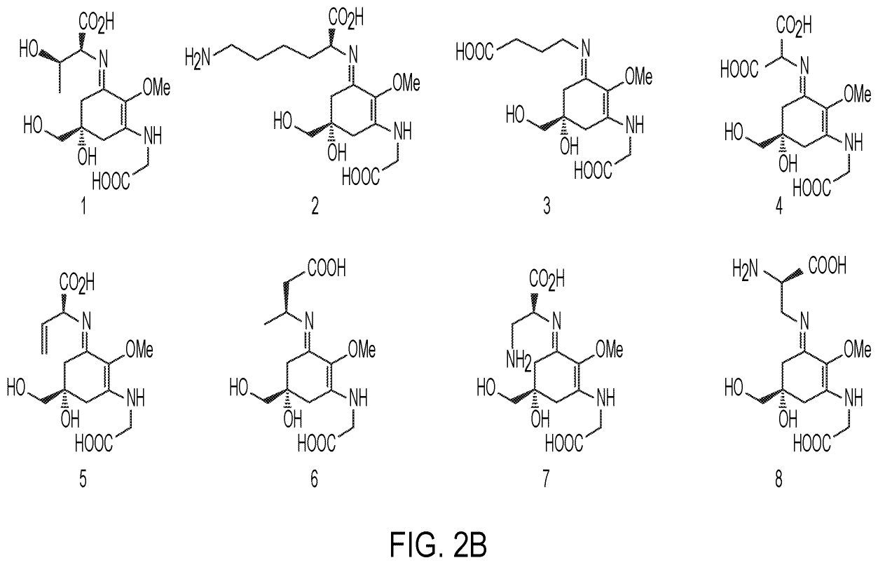

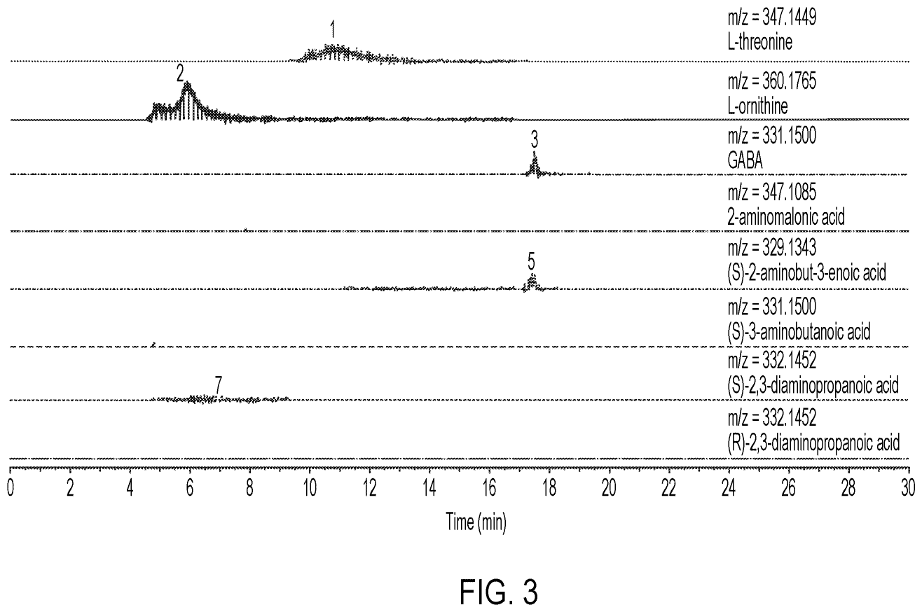

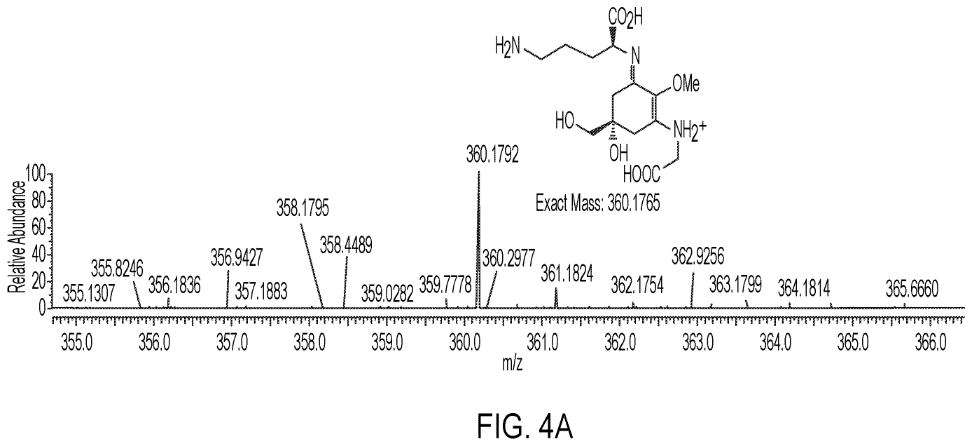

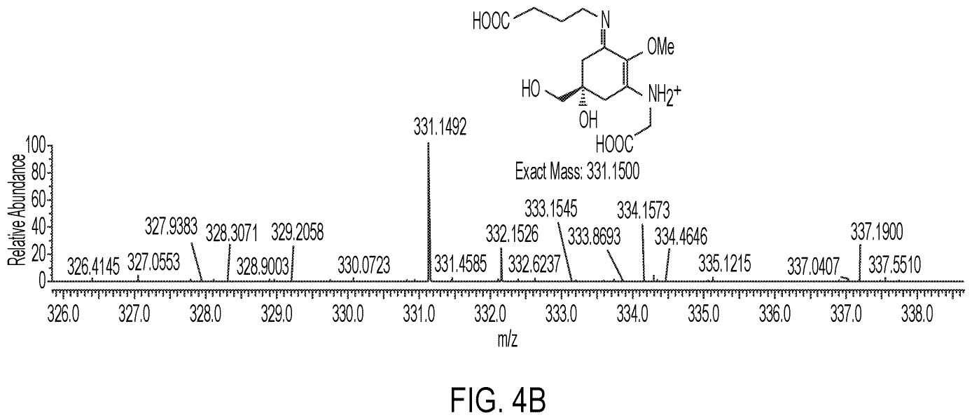

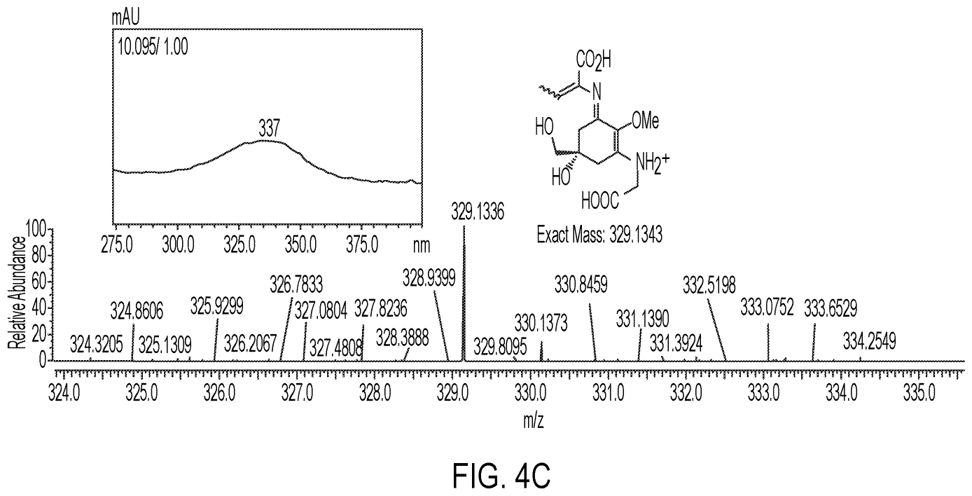

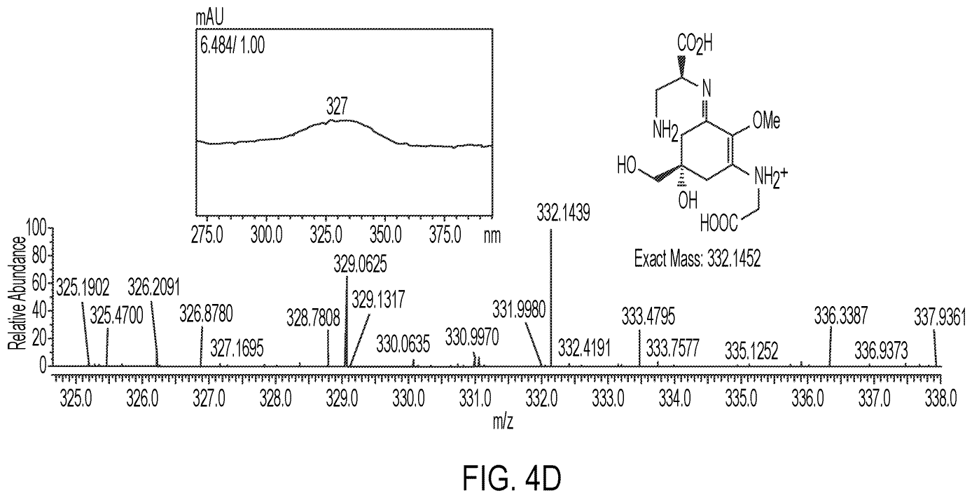

Seven amino acids along with L-threonine as the positive control were tested (FIG. 2A). The chemical structures of corresponding di-substituted MAA analogs were listed in FIG. 2B. The reaction solution contained 50 mM HEPES, pH 8.0, 10 mM MgCl₂, 50 uM mycosporine-glycine (MG), 5 mM amino acid substrates, and 5 mM ATP. The reactions were initiated by adding 0.5 uM MysD and then carried out at room temperature for 4 hours. The reactions were then terminated and centrifuged. The supernatants were analyzed by LC-HRMS, and the extracted ion chromatogram (EIC) traces indicated that, in addition to L-threonine, four unnatural amino acid substrates could be used by MysD to produce unnatural MAA products (FIG. 3), including L-ornithine, γ-aminobutyric acid, (S)-2-aminobut-3-enoic acid, and (S)-2,3-diaminopropionic acid. The structures of the new MAA analogs were validated by HRMS analysis (FIG. 4: Compound 2, observed [M+H]⁺ m/z 360.1792, calculated [M+H]⁺360.1765; Compound 3, observed [M+H]⁺ m/z 331.1492, calculated [M+H]⁺331.1500; Compound 5, observed [M+H]⁺ m/z 329.1336, calculated [M+H]⁺ 329.1343; Compound 7, observed [M+H]⁺ m/z 332.1439, calculated [M+H]⁺332.1452). Of note, the maximal absorption wavelengths of compounds 5 and 7 were shifted to 337 nm and 327 nm, respectively, instead of the 334 nm for the natural MAA porphyra-334 (FIG. 4C-D). These results indicate that the incorporation of unnatural amino acid moiety on the C1 could lead to the generation of MAA analogs with a broader UV coverage, aiding their applications in UV protection. MysD was not able to take (R)-2,3-diaminopropionic acid as substrate, suggesting its selection toward S-amine at the substrate C2.

### Example 2: Bioinformatic Analysis of MysD.

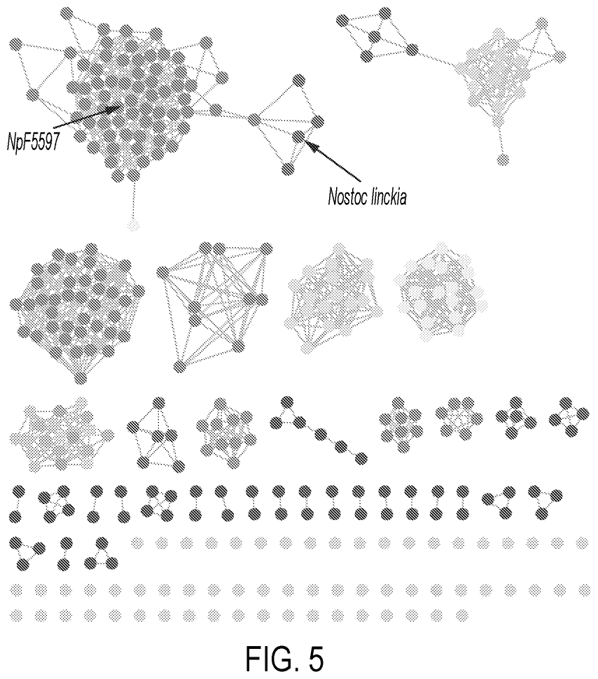

MysD from Nostoc linckia NIES-25 demonstrates substantial substrate flexibility in synthesizing MAA analogs, and its homologs can possess different substrate scopes. To examine the functional distribution of its homologs, sequence similarity network (SSN) analysis of 249 MysD homologs only from cyanobacterial genomes was performed. When cut-off threshold was set at 172 (corresponding to E-value<1e-160), the MysD homologs were separated into different clusters and singletons (FIG. 5). MysD from Nostoc linckia NIES-25 sits in the same cluster as the first reported MysD Npf5597 but in a distinct clade. This result aligns well with the results of their biochemical studies as these two MysD homologs have different substrate scopes. MysD of Nostoc linckia NIES-25 is a highly flexible enzyme with L-threonine as the best substrate, while Npf5597 favors L-serine over L-glycine and L-threonine (doi: 10.1128/JB.05730-11). Importantly, this result indicates that MysD homologs in different clusters can possess a wide range of substrate flexibility and they can be used to synthesize new unnatural MAA analogs.

### Example 3: Structure Guided Mutagenesis of MysD Improves Catalytic Efficiency.

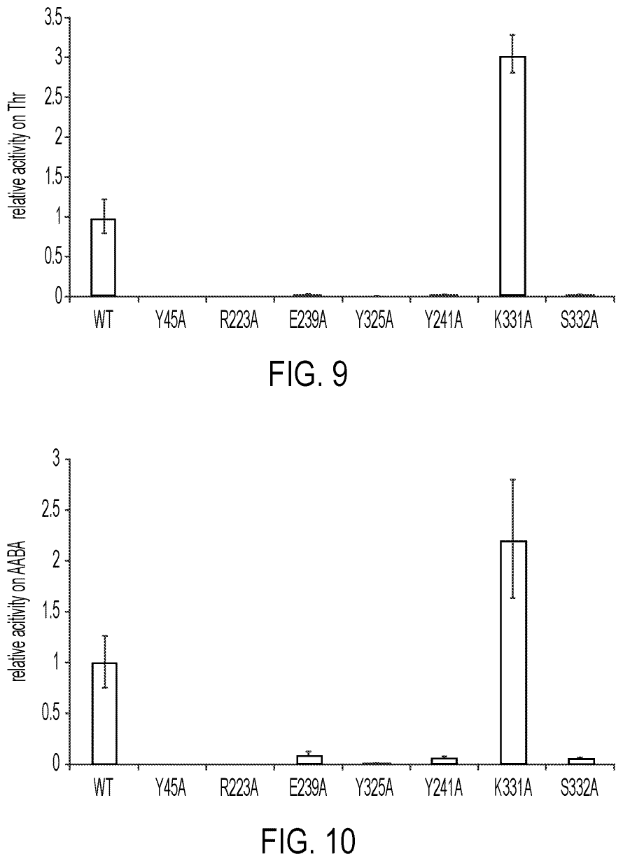

Based on the crystal structure of MysD docked with ATP, MG and Thr, several residues potentially important for substrate binding were selected for mutagenesis studies. Generated mutants included Y45A, R223A, E239A, Y241A, Y325A, K331A, and S332A. The relative activity of these MysD mutants was tested using L-Thr or α-aminobutyric acid (AABA) as the amino acid substrate. The results showed that most of these mutants lost the majority of the catalytic ability to both substrates (FIGS. 9 & 10), supporting their importance in substrate binding and/or proper enzyme folding. Surprisingly, the MysD K331A mutant showed significantly improved activity compared to the WT enzyme (3.0-fold activity on Thr and 2.2-fold activity on AABA). These results suggested the engineering of MysD could enable a more effective synthesis of MAA analogs and potentially more analogs.

### Example 4: Experimental Procedures.

General Experimental Procedures. Molecular biology reagents and chemicals were purchased from Thermo Scientific, NEB, Fisher Scientific or Sigma-Aldrich. GeneJET Plasmid Miniprep Kit and GeneJETGel Extraction Kit (Thermo Scientific) were used for plasmid preparation and DNA purification, respectively. E. coli DH5a (Agilent) was used for routine cloning studies and E. coli BL21-gold (DE3) (Agilent) was used for protein expression and heterologous production. The cyanobacterial strain Nostoc linkia NIES-25 was obtained from National Institute for Environmental Studies, Japan. DNA sequencing was performed with GENEWIZ or Eurofins. A Shimadzu Prominence UHPLC system (Kyoto, Japan) coupled with a PDA detector was used for HPLC analysis. NMR spectra were recorded in D₂O on a Bruker 600 MHz spectrometer located in the AMRIS facility at the University of Florida, Gainesville, FL, USA. Spectroscopy data were collected using Topspin 3.5 software. HRMS data were generated on a Thermo Fisher Q Exactive Focus mass spectrometer equipped with an electrospray probe on Universal Ion Max API source.

MysD Expression and Purification. The mysD gene was amplified from the isolated genomic DNA of Nostoc linkia NIES-25 and inserted into the NdeI/XhoI sites of pET28b, and the resultant construct pET28b-mysD was transformed into E. coli BL21-gold (DE3) for the expression of recombinant N-His₆-tagged MysD. Protein expression was carried out in 500 mL Luria-Bertani broth supplemented with 50 μg/mL kanamycin (37° C., 225 rpm).

When the cell culture OD₆₀₀ reached 0.5, IPTG (final concentration 0.1 mM) was added to the culture to induce gene expression (18° C., 180 rpm, 20 h). The cells were harvested by centrifugation (6000 rpm, 20 min), and collected cell pellets were resuspended in the lysis buffer (25 mM Tris-Cl, pH 8.0, 100 mM NaCl, 1 mM β-mercaptoethanol and 10 mM imidazole) and lysed by sonication on ice (10 s pulse and 20 s rest, 1 min in total).

Following centrifugation (15000 rpm, 4° C., 30 min), recombinant MysD was purified by the HisTrap Ni-NTA affinity column (GE Healthcare). N-His₆-tagged MysD was eluted using a 0-100% B gradient in 15 min at the flow rate of 2 mL/min, using A buffer (25 mM Tris-Cl, pH 8.0, 250 mM NaCl, 1 mM β-mercaptoethanol and 30 mM imidazole) and B buffer (25 mM Tris-Cl, pH 8.0, 250 mM NaCl, 1 mM β-mercaptoethanol and 300 mM imidazole). Fractions with recombinant MysD were collected, concentrated, and buffer-exchanged into storage buffer (50 mM Tris-Cl, pH 8.0, 10% glycerol). The purity of the recombinant protein was analyzed on SDS-PAGE and the concentration was determined by NanoDrop.

In Vitro MysD Reactions. MG was purified from extracts of E. coli expressing MysAB2C by HPLC and used as the substrate for the MysD reactions. The quality of MG was calculated based on its extinction coefficient (28,100 M⁻¹cm⁻¹). The MysD reactions included MG (50 μM), Mg²⁺ (10 mM), unnatural amino acid substrates (5 mM), and ATP (5 mM) in 50 mM HEPES, pH 8.0. The reactions were initiated by adding MysD (0.5 μM) and then incubated at room temperature for 4 hours. All reactions were quenched by heat inactivation at 95° C. for 10 min. After centrifugation at 20,000×g for 15 min, the clear supernatants were collected for LC-HRMS analysis.

To explore the substrate scope of MysD, the natural amino acid L-threonine and the unnatural amino acids L-ornithine, 4-aminobutanoic acid, 2-aminomalonic acid, (S)-2-aminobut-3-enoic acid, (S)-3-aminobutanoic acid, (S)-2,3-diaminopropanoic acid, and (R)-2,3-diaminopropanoic acid (each at 5 mM) were each screened in the above reaction mixtures under the optimal conditions. The reactions were terminated and then analyzed in the HPLC and/or LC-MS analysis.

HPLC and LC-MS Analysis. Samples were analyzed on a Shimadzu Prominence UHPLC system (Kyoto, Japan) coupled with a PDA detector. Unless stated elsewhere, the following HPLC procedure was performed. The compounds were separated on a Phenomenex Luna C8 column (4.6×250 mm, 5 μm) using the following HPLC program: 2% B for 15 min, 2-90% B gradient in 2 min, 90% B in 2 min, 90-2% in 2 min, and re-equilibration in 2% B for 6 min. The A phase was water with 0.1% formic acid and the B phase was methanol. The flow rate was set at 0.5 mL/min. LC-HRMS and HRMS/MS experiments were conducted on a Thermo Scientific Q Exactive Focus mass spectrometer with a Dionex Ultimate RSLC 3000 uHPLC system, equipped with the H-ESI II probe on an Ion Max API Source. Methanol (B)/water (A) containing 0.1% formic acid were used as mobile phases. The eluents from the first 3 min were diverted to waste by a diverting valve. MS1 signals were acquired under the Full MS positive ion mode, covering a mass range of m/z 150-2000, with resolution at 35 000 and AGC target at 1×10⁶.

Bioinformatic Analysis of MysD. Protein sequences from 595 cyanobacteria genomes were obtained by protein BLAST search against the NCBI non-redundant protein database (E-value<1e-5) using query sequences for Nostoc linckia NIES-25 MysD (accession: WP_096541781.1). After filtering sequence length to obtain proteins with 300-400 amino acids, 249 MysD homologs were retrieved. The SSN of the MysD homologs was generated by the EFI-Enzyme Similarity Tool (https://efi.igb.illinois.edu/efi-est/) with a filter-value at 172.

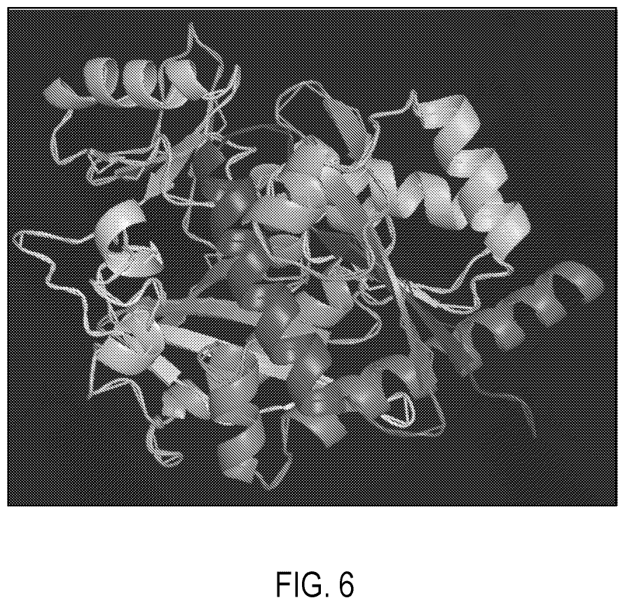

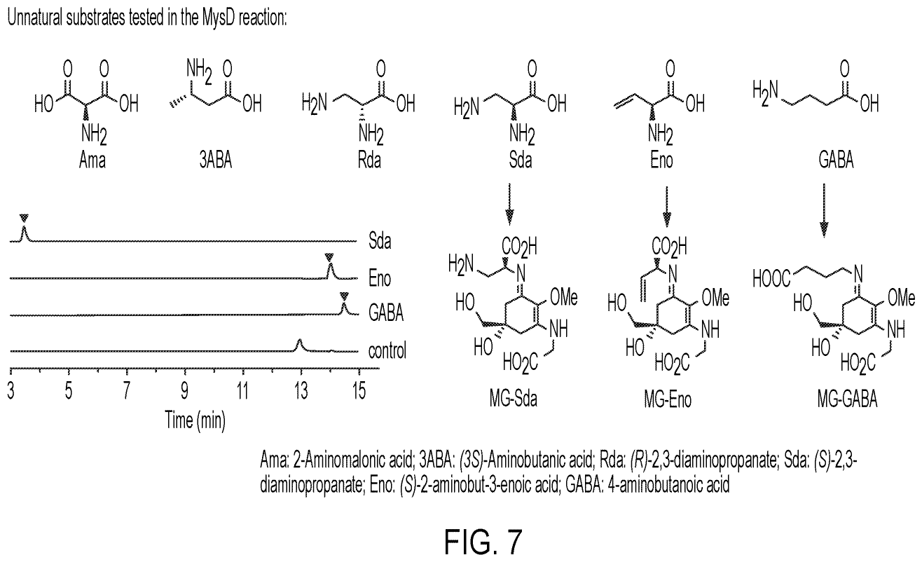

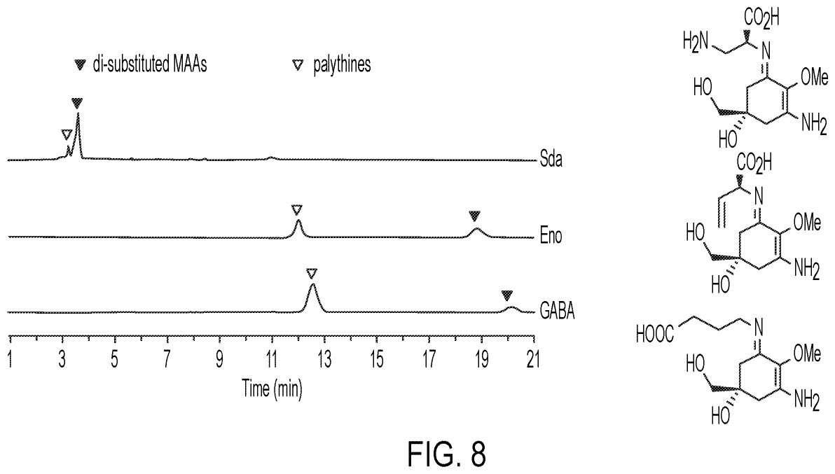

Protein engineering has become a widely used approach to tailor enzyme substrate scopes for chemical synthesis. The protein structure can assist rational engineering of proteins. Since MysD 3-D structure is not available, the structure of MysD of Nostoc linckia NIES-25 was predicted using RoseTTAFold (FIG. 6) (doi: 10.1126/science.abj8754).

### REFERENCES

1. Rogers, H. W.; Weinstock, M. A.; Feldman, S. R.; Coldiron, B. M., Incidence estimate of nonmelanoma skin cancer (keratinocyte carcinomas) in the U.S. population, 2012. JAMA Dermatol. 2015, 151 (10), 1081-1086.
2. Siegel, R. L.; Miller, K. D.; Fuchs, H. E.; Jemal, A., Cancer statistics, 2021. CA: Cancer J. Clin. 2021, 71 (1), 7-33.
3. Moan, J.; Grigalavicius, M.; Baturaite, Z.; Dahlback, A.; Juzeniene, A., The relationship between UV exposure and incidence of skin cancer. Photodermatol. Photoimmunol. Photomed. 2015, 31 (1), 26-35.
4. Armstrong, B. K.; Kricker, A., How much melanoma is caused by sun exposure. Melanoma Res. 1993, 3 (6), 395-401.
5. Holick, M. F., Biological effects of sunlight, ultraviolet radiation, visible light, infrared radiation and vitamin D for health. Anticancer Res. 2016, 36 (3), 1345-1356.
6. Ghiasvand, R.; Weiderpass, E.; Green, A. C.; Lund, E.; Veierod, M. B., Sunscreen use and subsequent melanoma risk: A population-based cohort study. J. Clin. Oncol. 2016, 34 (33), 3976-3983.
7. Latha, M. S.; Martis, J.; Shobha, V.; Sham Shinde, R.; Bangera, S.; Krishnankutty, B.; Bellary, S.; Varughese, S.; Rao, P.; Naveen Kumar, B. R., Sunscreening agents: a review. J. Clin. Aesthet. Dermatol. 2013, 6 (1), 16-26.
8. Krause, M.; Klit, A.; Jensen, M. B.; Soeborg, T.; Frederiksen, H.; Schlumpf, M.; Lichtensteiger, W.; Skakkebaek, N. E.; Drzewiecki, K. T., Sunscreens: are they beneficial for health? An overview of endocrine disrupting properties of UV-filters. Int. J. Androl. 2012, 35 (3), 424-436.
9. Ruszkiewicz, J. A.; Pinkas, A.; Ferrer, B.; Peres, T. V.; Tsatsakis, A.; Aschner, M., Neurotoxic effect of active ingredients in sunscreen products, a contemporary review. Toxicol. Rep. 2017, 4, 245-259.
10. Matta, M. K.; Zusterzeel, R.; Pilli, N. R.; Patel, V.; Volpe, D. A.; Florian, J.; Oh, L.; Bashaw, E.; Zineh, I.; Sanabria, C.; Kemp, S.; Godfrey, A.; Adah, S.; Coelho, S.; Wang, J.; Furlong, L. A.; Ganley, C.; Michele, T.; Strauss, D. G., Effect of sunscreen application under maximal use conditions on plasma concentration of sunscreen active ingredients a randomized clinical trial. JAMA 2019, 321 (21), 2082-2091.
11. Schneider, S. L.; Lim, H. W., Review of environmental effects of oxybenzone and other sunscreen active ingredients. J. Am. Acad. Dermatol. 2019, 80 (1), 266-271.
12. Pandika, M., Looking to nature for new sunscreens. ACS Cent. Sci. 2018, 4 (7), 788-790.
13. Saewan, N.; Jimtaisong, A., Natural products as photoprotection. J. Cosmet. Dermatol. 2015, 14 (1), 47-63.
14. Kageyama, H.; Waditee-Sirisattha, R., Antioxidative, anti-inflammatory, and anti-aging properties of mycosporine-like amino acids: Molecular and cellular mechanisms in the protection of skin-aging. Mar. Drugs 2019, 17 (4), 222. doi: 10.3390/md17040222.
15. Losantos, R.; Funes-Ardoiz, I.; Aguilera, J.; Herrera-Ceballos, E.; Garcia-Iriepa, C.; Campos, P. J.; Sampedro, D., Rational design and synthesis of efficient sunscreens to boost the solar protection factor. Angew. Chem. Int. Ed. Engl. 2017, 56 (10), 2632-2635.
16. Carreto, J. I.; Carignan, M. O., Mycosporine-like amino acids: Relevant secondary metabolites. Chemical and ecological aspects. Mar. Drugs 2011, 9 (3), 387-446.
17. M. Bandaranayake, W., Mycosporines: are they nature's sunscreens? Nat. Prod. Rep. 1998, 15 (2), 159-172.
18. Sinha, R. P.; Singh, S. P.; Hader, D. P., Database on mycosporines and mycosporine-like amino acids (MAAs) in fungi, cyanobacteria, macroalgae, phytoplankton and animals. J. Photochem. Photobiol. B 2007, 89 (1), 29-35.
19. Kicklighter, C. E.; Kamio, M.; Nguyen, L.; Germann, M. W.; Derby, C. D., Mycosporine-like amino acids are multifunctional molecules in sea hares and their marine community. Proc Natl Acad Sci USA 2011, 108 (28), 11494-11499.
20. Nazifi, E.; Wada, N.; Yamaba, M.; Asano, T.; Nishiuchi, T.; Matsugo, S.; Sakamoto, T., Glycosylated porphyra-334 and palythine-threonine from the terrestrial cyanobacterium Nostoc commune. Mar. Drugs 2013, 11 (9), 3124-3154.
21. D'Agostino, P. M.; Javalkote, V. S.; Mazmouz, R.; Pickford, R.; Puranik, P. R.; Neilan, B. A., Comparative profiling and discovery of novel glycosylated mycosporine-like amino acids in two strains of the cyanobacterium Scytonema cf. crispum. Appl. Environ. Microbiol. 2016, 82 (19), 5951-5959.
22. Akio, F.; Takeshi, M.; Isami, T.; Isao, S., The crystal and molecular structure of palythine trihydrate. Bull. Chem. Soc. Jpn. 1980, 53 (2), 319-323.
23. Daisuke, U.; Chuji, K.; Akio, W.; Yoshimasa, H., Crystal and molecule structure of palythiene possessing a novel 360 nm chromophore. Chem. Lett. 1980, 9 (6), 755-756.
24. Klisch, M.; Richter, P.; Puchta, R.; Häder, D.-P.; Bauer, W., The stereostructure of porphyra-334: An experimental and calculational NMR investigation. Evidence for an efficient ‘proton sponge’. Helv. Chim. Acta 2007, 90 (3), 488-511.
25. White, J. D.; Cammack, J. H.; Sakuma, K.; Rewcastle, G. W.; Widener, R. K., Transformations of quinic acid. Asymmetric synthesis and absolute configuration of mycosporin I and mycosporin-gly. J. Org. Chem. 1995, 60 (12), 3600-3611.
26. Yang, G.; Cozad, M. A.; Holland, D. A.; Zhang, Y.; Luesch, H.; Ding, Y., Photosynthetic production of sunscreen shinorine using an engineered cyanobacterium. ACS Synth. Biol. 2018, 7 (2), 664-671.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

1. A method for producing a compound, comprising:
a) culturing a recombinant microorganism under conditions suitable for production of the compound; and
b) isolating the compound from the recombinant microorganism,
wherein the recombinant microorganism comprises a heterologous nucleic acid encoding one or more mycosporine-like amino acid (MAA) biosynthetic enzymes, wherein the one or more MAA biosynthetic enzymes comprise a D-alanine-D-alanine ligase (MysD), or a homolog thereof.

2. The method of claim 1, wherein the D-alanine-D-alanine ligase comprises an amino acid sequence of SEQ ID NO: 1, or an amino acid sequence at least 70%, at least 75%, at least 80%, at least 85%, at least 90%, at least 95%, or at least 99% identical to the amino acid sequence of SEQ ID NO: 1.

3. The method of claim 1 or 2, wherein the one or more MAA biosynthetic enzymes further comprise a phytanoyl-CoA dioxygenase (MysH), or a homolog thereof.

4. The method of claim 3, wherein the phytanoyl-CoA dioxygenase comprises an amino acid sequence of any one of SEQ ID NOs: 2-12, or an amino acid sequence at least 70%, at least 75%, at least 80%, at least 85%, at least 90%, at least 95%, or at least 99% identical to the amino acid sequence of any one of SEQ ID NOs: 2-12.

5. The method of any one of claims 1-4, wherein the one or more MAA biosynthetic enzymes further comprise an ATP-grasp enzyme (MysC), or a homolog thereof.

6. The method of claim 5, wherein the ATP-grasp enzyme comprises an amino acid sequence of any one of SEQ ID NOs: 13-105, or an amino acid sequence at least 70%, at least 75%, at least 80%, at least 85%, at least 90%, at least 95%, or at least 99% identical to the amino acid sequence of any one of SEQ ID NOs: 13-105.

7. The method of any one of claims 1-6, wherein the one or more biosynthetic enzymes further comprise one or more enzymes selected from the group consisting of a demethyl-4-deoxygadusol synthase (MysA), an O-methyltransferase (MysB), and a non-ribosomal peptide synthetase (NRPS)-like enzyme (MysE).

8. The method of any one of claims 1-7, wherein the compound is a mycosporine-like amino acid.

9. The method of any one of claims 1-8, wherein the compound has UV-modulating activity.

10. The method of claim 9, wherein the UV-modulating activity comprises absorption of UV wavelengths between 280 and 400 nm.

11. The method of any one of claims 1-10, wherein the compound is of Formula (I), or a salt thereof: 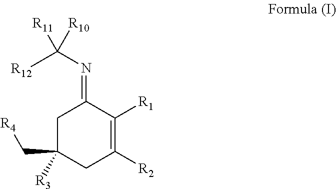
wherein:
each of R₁, R₂, R₃, and R₄ is independently selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl; R₁₀ is selected from the group consisting of hydrogen and —CO₂R₁₃, wherein R₁₃ may be selected from the group consisting of hydrogen and optionally substituted alkyl; and
each of R₁₁ and R₁₂ is independently selected from the group consisting of hydrogen, optionally substituted alkyl, optionally substituted alkenyl, or R₁₁ and R₁₂ are taken together to form an optionally substituted alkenyl group;
provided that if R₁₀ is —CO₂R₁₃ and R₁₁ is hydrogen, R₁₂ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂, 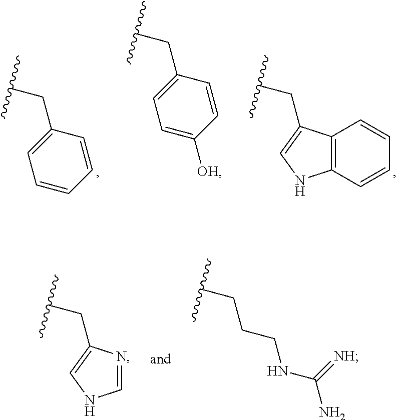 [SMILES: *Cc1ccccc1 ; *Cc1ccc(O)cc1 ; *Cc1c[nH]c2ccccc12 ; *Cc1c[nH]cn1 ; *CCCNC(=N)N]
and
provided that if R₁₀ is —CO₂R₁₃ and R₁₂ is hydrogen, R₁₁ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

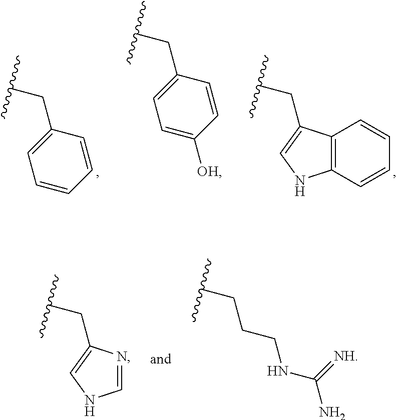 [SMILES: *Cc1ccccc1 ; *Cc1ccc(O)cc1 ; *Cc1c[nH]c2ccccc12 ; *Cc1c[nH]cn1 ; *CCCNC(=N)N]

12. The method of claim 11, wherein R₁ is —OR^{a}, wherein R^{a} is optionally substituted C₁₋₆ alkyl.

13. The method of claim 12, wherein R₁ is —OCH₃.

14. The method of any one of claims 11-13, wherein R₂ is —NH₂.

15. The method of any one of claims 11-14, wherein R₃ is —OH.

16. The method of any one of claims 11-15, wherein R₄ is —OH.

17. The method of any one of claims 11-16, wherein R₁₀ is —CO₂H, R₁₁ is hydrogen, and R₁₂ is

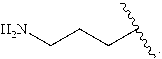 [SMILES: *CCCN]

18. The method of any one of claims 11-16, wherein R₁₀ is hydrogen, R₁₁ is hydrogen, and R₁₂ is

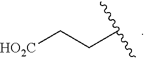 [SMILES: *CCC(=O)O]

19. The method of any one of claims 11-16, wherein R₁₀ is —CO₂H, and R₁₁ and R₁₂ are taken together to form

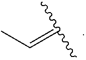 [SMILES: *=CC]

20. The method of any one of claims 11-16, wherein R₁₀ is —CO₂H, R₁₁ is hydrogen, and R₁₂ is

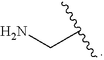 [SMILES: *CN]

21. The method of claim 11, wherein the compound is of the formula:

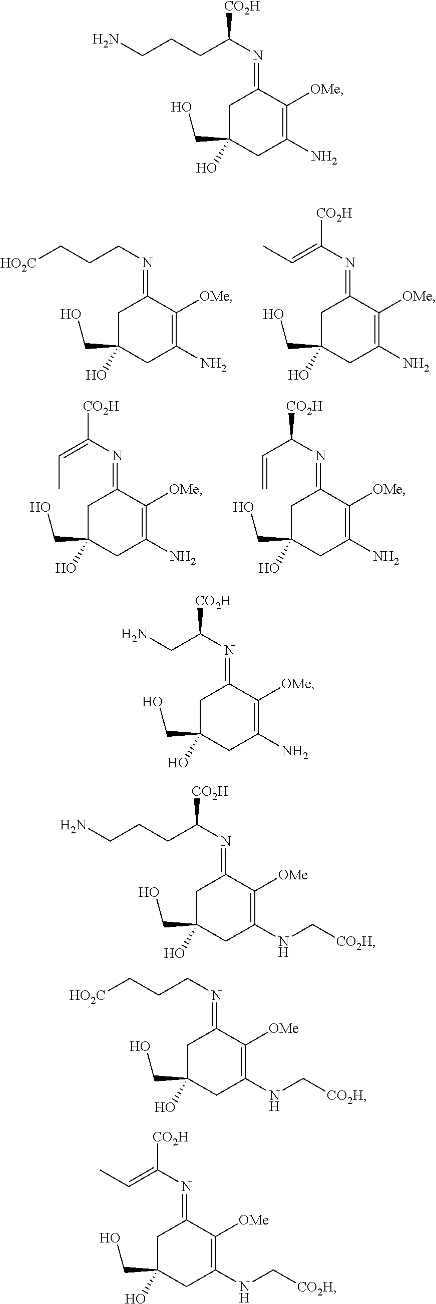

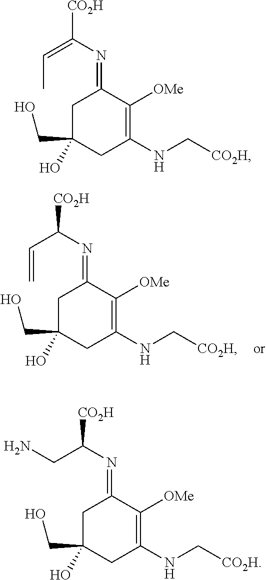

22. The method of any one of claims 1-21, further comprising providing a substrate of the one or more mycosporine-like amino acid (MAA) biosynthetic enzymes to the recombinant microorganism.

23. The method of claim 22, wherein the substrate is a compound of Formula (II), or a salt thereof: 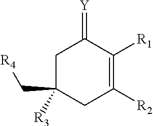
wherein:
each of R₁, R₂, R₃, and R₄ is independently selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl; and
Y is ═O or 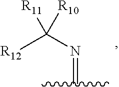
wherein:
R₁₀ is selected from the group consisting of hydrogen and —CO₂R₁₃, wherein R₁₃ may be selected from the group consisting of hydrogen and optionally substituted alkyl; and
each of R₁₁ and R₁₂ is independently selected from the group consisting of hydrogen, optionally substituted alkyl, optionally substituted alkenyl, or R₁₁ and R₁₂ are taken together to form an optionally substituted alkenyl group;
provided that if R₁₀ is —CO₂R₁₃ and R₁₁ is hydrogen, R₁₂ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂, 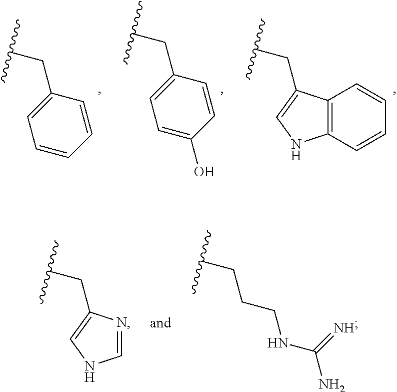 [SMILES: *Cc1ccccc1 ; *Cc1ccc(O)cc1 ; *Cc1c[nH]c2ccccc12 ; *Cc1c[nH]cn1 ; *CCCNC(=N)N]
and
provided that if R₁₀ is —CO₂R₁₃ and R₁₂ is hydrogen, R₁₁ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

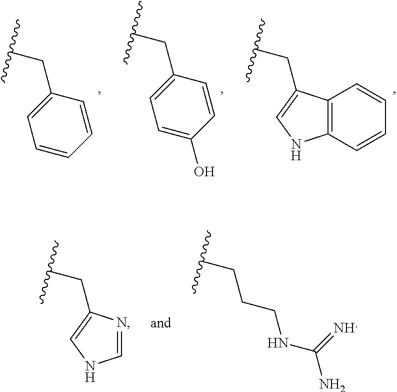 [SMILES: *Cc1ccccc1 ; *Cc1ccc(O)cc1 ; *Cc1c[nH]c2ccccc12 ; *Cc1c[nH]cn1 ; *CCCNC(=N)N]

24. The method of claim 23, wherein R₁ is —OH.

25. The method of claim 23, wherein R₁ is —OCH₃.

26. The method of any one of claims 23-25, wherein R₂ is —OH.

27. The method of any one of claims 23-25, wherein R₂ is —NH₂.

28. The method of any one of claims 23-25, wherein R₂ is —(NH)R^{b}, wherein R^{b} is optionally substituted alkyl.

29. The method of claim 28, wherein R₂ is —NHCH₂CO₂H.

30. The method of any one of claims 23-29, wherein R₃ is —OH.

31. The method of any one of claims 23-30, wherein R₄ is —OH.

32. The method of any one of claims 23-31, wherein Y is O.

33. The method of any one of claims 23-31, wherein Y is

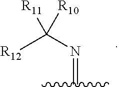

34. The method of claim 33, wherein R₁₀ is —CO₂H, R₁₁ is hydrogen, and R₁₂ is

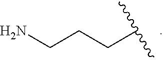 [SMILES: *CCCN]

35. The method of claim 33, wherein R₁₀ is hydrogen, R₁₁ is hydrogen, and R₁₂ is

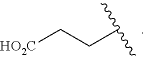 [SMILES: *CCC(=O)O]

36. The method of claim 33, wherein R₁₀ is —CO₂H, and R₁₁ and R₁₂ are taken together to form

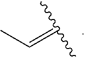 [SMILES: *=CC]

37. The method of claim 33, wherein R₁₀ is —CO₂H, R₁₁ is hydrogen, and R₁₂ is

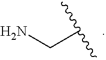 [SMILES: *CN]

38. The method of claim 23, wherein the substrate is of the formula:

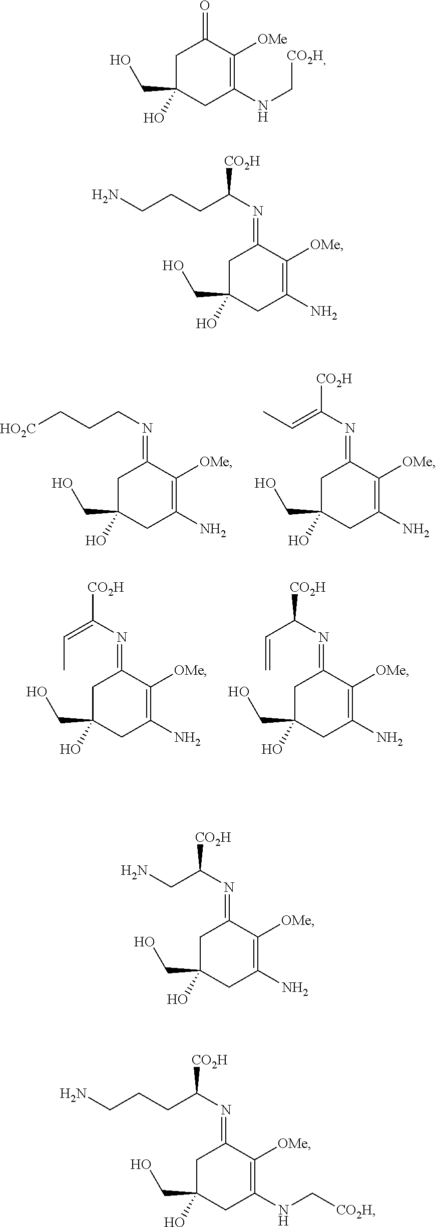

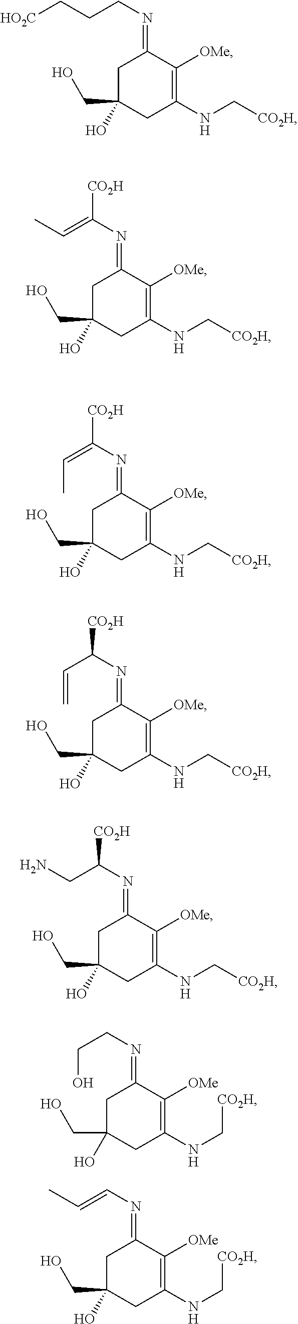

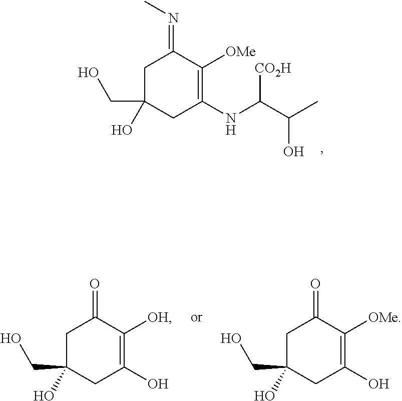 [SMILES: C/N=C1\CC(O)(CO)CC(NC(C(=O)O)C(C)O)=C1OC ; O=C1C[C@](O)(CO)CC(O)=C1O ; "or" ; COC1=C(O)C[C@@](O)(CO)CC1=O]

39. The method of any one of claims 1-38, wherein the recombinant microorganism is a species of bacteria or yeast.

40. The method of claim 39, wherein the bacteria is a species of cyanobacteria.

41. The method of claim 39, wherein the bacteria is a species from the human microbiome.

42. The method of claim 39, wherein the bacteria is E. coli.

43. A recombinant microorganism comprising a heterologous nucleic acid encoding one or more mycosporine-like amino acid (MAA) biosynthetic enzymes, wherein the one or more MAA biosynthetic enzymes comprise a D-alanine-D-alanine ligase (MysD), or a homolog thereof.

44. A method of producing a compound, comprising:
a) culturing the recombinant microorganism of claim 43 under conditions suitable for production of the compound; and
b) isolating the compound from the recombinant microorganism.

45. A composition comprising a compound produced by the method of any one of claim 1-42 or 44 and optionally an excipient.

46. The composition of claim 45, wherein the composition is for topical administration.

47. The composition of claim 45 or 46, wherein the composition is formulated as a sunscreen.

48. The composition of claim 45 or 46, wherein the composition is formulated as a cosmetic.

49. A method of making the composition of any one of claims 45-48, comprising:
a) culturing a recombinant microorganism under conditions suitable for production of the compound;
b) isolating the compound from the recombinant microorganism,
wherein the recombinant microorganism comprises a heterologous nucleic acid encoding one or more mycosporine-like amino acid (MAA) biosynthetic enzymes, wherein the one or more MAA biosynthetic enzymes comprise a D-alanine-D-alanine ligase (MysD), or a homolog thereof; and
c) adding the compound to one or more excipients to produce the composition.

50. A method of administering a compound, comprising applying the composition of any one of claims 45-48 to a subject.

51. A method of preventing sunburn, comprising applying the composition of any one of claims 45-48 on the skin of a subject in need thereof.

52. A method of preventing cancer, comprising applying the composition of any one of claims 45-48 on the skin of a subject in need thereof.

53. A method of preventing or treating a chronic inflammatory disease, comprising administering the composition of any one of claims 45-48 to a subject in need thereof.

54. A compound produced by:
a) culturing a recombinant microorganism under conditions suitable for production of the compound; and
b) isolating the compound from the recombinant microorganism,
wherein the recombinant microorganism comprises a heterologous nucleic acid encoding one or more mycosporine-like amino acid (MAA) biosynthetic enzymes, wherein the one or more MAA biosynthetic enzymes comprise a D-alanine-D-alanine ligase (MysD), or a homolog thereof.

55. The compound of claim 54, wherein the compound is of Formula (I), or a salt thereof: 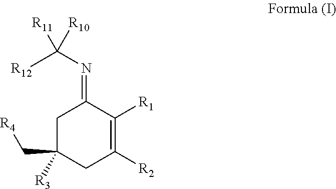
wherein:
each of R₁, R₂, R₃, and R₄ is independently selected from the group consisting of —OR^{a}, —(NH)R^{b}, and —N(R^{b})₂, wherein each instance of R^{a} is independently hydrogen or optionally substituted C₁₋₆ alkyl and each instance of R^{b} is independently hydrogen or optionally substituted C₁₋₆ alkyl;
R₁₀ is selected from the group consisting of hydrogen and —CO₂R₁₃, wherein R₁₃ may be selected from the group consisting of hydrogen and optionally substituted alkyl;
each of R₁₁ and R₁₂ is independently selected from the group consisting of hydrogen, optionally substituted alkyl, optionally substituted alkenyl, or R₁₁ and R₁₂ are taken together to form an optionally substituted alkenyl group;
provided that if R₁₀ is —CO₂R₁₃ and R₁₁ is hydrogen, R₁₂ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂, 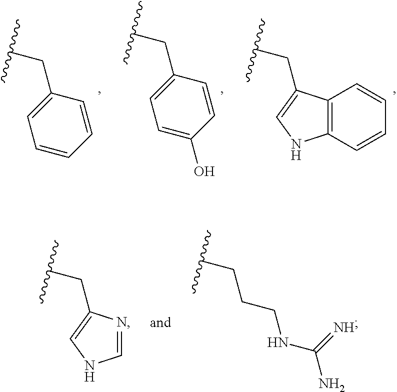 [SMILES: *Cc1ccccc1 ; *Cc1ccc(O)cc1 ; *Cc1c[nH]c2ccccc12 ; *Cc1c[nH]cn1 ; *CCCNC(=N)N]
and
provided that if R₁₀ is —CO₂R₁₃ and R₁₂ is hydrogen, R₁₁ is not selected from the group consisting of hydrogen, —CH₃, —CH(CH₃)₂, —CH₂CH(CH₃)₂, —CH(CH₃)CH₂CH₃, —CH₂OH, —CH(OH)CH₃, —CH₂C(O)NH₂, —CH₂CH₂C(O)NH₂, —CH₂SH, —CH₂CH₂SCH₃, —CH₂CO₂H, —CH₂CH₂CO₂H, —CH₂CH₂CH₂CH₂NH₂,

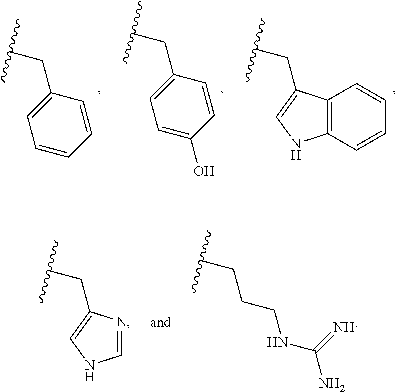 [SMILES: *Cc1ccccc1 ; *Cc1ccc(O)cc1 ; *Cc1c[nH]c2ccccc12 ; *Cc1c[nH]cn1 ; *CCCNC(=N)N]

56. The compound of claim 55, wherein R₁ is —OR^{a}, wherein R^{a} is optionally substituted C₁₋₆ alkyl.

57. The compound of claim 56, wherein R₁ is —OCH₃.

58. The compound of any one of claims 55-57, wherein R₂ is —NH₂.

59. The compound of any one of claims 55-58, wherein R₃ is —OH.

60. The compound of any one of claims 55-59, wherein R₄ is —OH.

61. The compound of any one of claims 55-60, wherein R₁₀ is —CO₂H, R₁₁ is hydrogen, and R₁₂ is

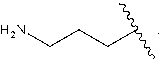 [SMILES: *CCCN]

62. The compound of any one of claims 55-60, wherein R₁₀ is hydrogen, R₁₁ is hydrogen, and R₁₂ is

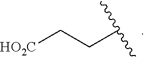 [SMILES: *CCC(=O)O]

63. The compound of any one of claims 55-60, wherein R₁₀ is —CO₂H, and R₁₁ and R₁₂ are taken together to form

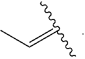 [SMILES: *=CC]

64. The compound of any one of claims 55-60, wherein R₁₀ is —CO₂H, R₁₁ is hydrogen, and R₁₂ is

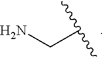 [SMILES: *CN]

65. The compound of any one of claims 55-64, wherein the compound is of the formula:

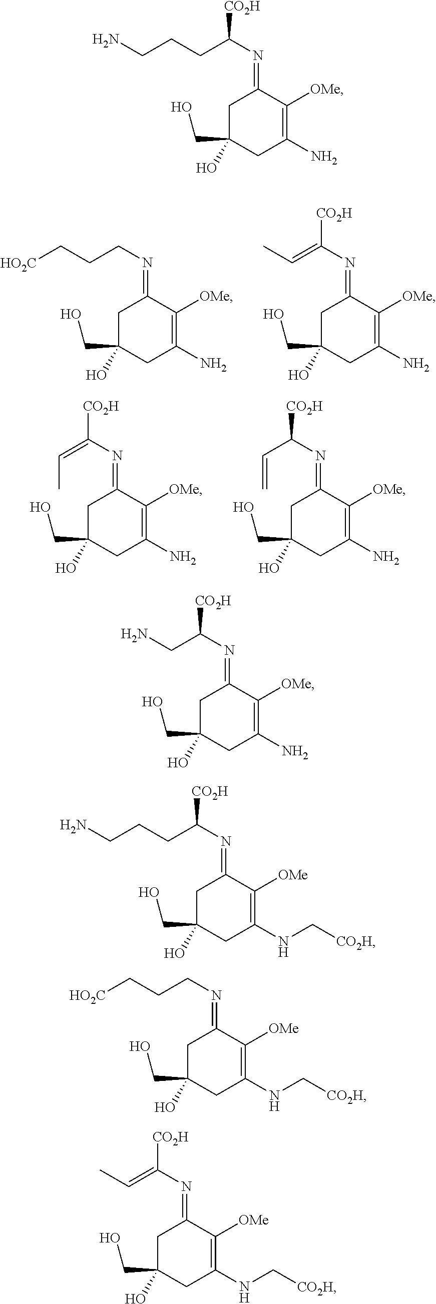

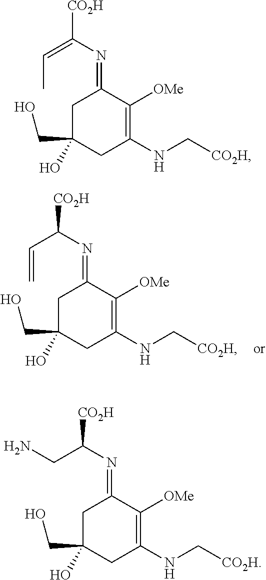

66. A composition comprising the compound of any one of claims 55-65, or a salt thereof.

67. The composition of claim 66, wherein the composition is for topical administration.

68. The composition of claim 66 or 67, wherein the composition is formulated as a sunscreen.

69. The composition of claim 66 or 67, wherein the composition is formulated as a cosmetic.

70. A method of administering a compound, comprising applying the composition of any one of claims 66-69 to a subject.

71. A method of preventing sunburn, comprising applying the composition of any one of claims 66-69 on the skin of a subject in need thereof.

72. A method of preventing cancer, comprising applying the composition of any one of claims 66-69 on the skin of a subject in need thereof.

73. A method of treating or preventing a chronic inflammatory disease, comprising applying the composition of any one of claims 66-69 on the skin of a subject in need thereof.
